(19) United States
(12) Patent Application Publication
(10) Pub. No.: US 2025/0025525 A1
(43) Pub. Date: Jan. 23, 2025

(54) OCTAPEPTIDE FOR TOPICAL APPLICATION

(71) Applicant: Alastin Skincare, Inc., Carlsbad, CA (US)

(72) Inventors: Alan David WIDGEROW, Carlsbad, CA (US); John A. GARRUTO, Carlsbad, CA (US)

(73) Assignee: Alastin Skincare, Inc., Carlsbad, CA (US)

(21) Appl. No.: 18/913,581

(22) Filed: Oct. 11, 2024

Related U.S. Application Data
(63) Continuation of application No. PCT/US2023/018348, filed on Apr. 12, 2023.
(60) Provisional application No. 63/330,752, filed on Apr. 13, 2022.

(51) Int. Cl. A61K 38/06; A61K 9/00; A61K 31/12; A61K 36/07; A61K 38/08; A61K 38/40; A61K 47/24

(52) U.S. Cl. CPC A61K 38/06; A61K 9/0014; A61K 31/12; A61K 36/07; A61K 38/08; A61K 38/40; A61K 47/24

(57) ABSTRACT

Described herein are compositions and methods comprising one or more peptides including an octapeptide with amino acid sequence features found in collagen and/or elastin.

### CROSS-REFERENCE

This application is a continuation of International Patent Application No. PCT/US2023/018348 filed Apr. 12, 2023, which application claims the benefit of U.S. Provisional Patent Application No. 63/330,752, filed on Apr. 13, 2022, both of which are hereby incorporated by reference in their entirety.

### REFERENCE TO SEQUENCE LISTING

The present application is filed along with a Sequence Listing in electronic format. The Sequence Listing is provided as a file entitled 105153-60012.xml, created on Oct. 10, 2024, which is 12,934 bytes in size. The information in the electronic format of the Sequence Listing is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Hyaluronic acid (HA) plays an important role in cellular and extracellular matrix (ECM) homeostasis in skin. HA is involved in regulating hydration, cell turnover, metabolism, tissue repair, nutrient exchange and protection against free radical damage. The HA-bound water in both the dermis and the epidermis is critical for skin hydration. While HA decreases with aging, HA content in skin in fact remains constant with age. The difference is that the HA becomes increasingly tissue-bound and more resistant to extraction. This HA encased within tissue proteins may be limited in its hydrating capacity and lower molecular weight (MW) HA present in these tissue-bound forms may be pro-inflammatory. Thus, a topical composition to accelerate synthesis of high MW HA would be beneficial in improving skin health.

Elastin is a key extracellular matrix (ECM) protein that provides stretch, recoil, resilience, and elasticity to tissues and organs, including skin. Normal levels of elastic fiber production, organization, and integration with other cutaneous extracellular matrix proteins, proteoglycans, and glycosaminoglycans, are key to maintaining healthy skin structure, function, and youthful appearance. Elastin production decreases with age, and elastin degrades upon exposure to environmental insults, particularly sun exposure, causing loss of skin structural integrity and contributing to sagging skin or crepiness and related undesirable changes in appearance. Disruption of elastic fiber networks also leads to sub-optimal wound healing, and the worsening structure and appearance of scars and stretch marks. Thus, topical compositions which can enhance production of elastic fibers would be highly advantageous for improving and/or restoring appearance, texture, resiliency, and wound-healing capabilities to the skin.

### SUMMARY

Skin aging is associated with a loss of skin moisture and reduced barrier function. Aged or damaged skin can present with various signs of inflammation, which may be visible as increased skin redness, increased visibility of fine blood vessels, and certain skin conditions including, but not limited to, rosacea. Environmental influences, such as excessive UV light exposure, may contribute to the visible, progressive changes seen in aged skin. Topical compositions and methods that promote intrinsic high MW HA production or increase elastin production in skin may be helpful to treat various skin conditions.

Often, improving skin hydration and reducing inflammation in skin may help to treat damaged, inflamed, or aging skin. Application of topical compositions with peptides have been used to treat damaged, inflamed, or aging skin.

Described herein are compositions and methods comprising a topical formulation and application of an octapeptide. In some embodiments, the compositions and methods described herein reduce skin inflammation, appearance of solar elastosis, and/or signs of skin aging. In some embodiments, compositions and methods described herein improve skin health, appearance, and hydration. In some embodiments, the compositions and methods described herein increase HA production and/or increase elastin production.

There remains a need to promote proper skin hydration and healthy skin tone, texture and elasticity in individuals. Skin aging is associated with a loss of skin moisture and dry skin results in reduced barrier function, as well as decreased elastin production and degradation of elastin fiber networks. Environmental influences, such as excessive UV light exposure, also contribute to the visible, progressive changes seen in aged skin such as accelerated development of fine lines and wrinkles as well as decreases in skin elasticity. There exists a need for topical compositions and methods that promote intrinsic high MW HA production within epidermal and dermal cells to improve skin health and which promote elastin production to restore or improve the appearance of aging or damaged skin.

In one aspect, which may be combined with any other aspect or embodiment, the present disclosure relates to a topical composition for stimulating synthesis of high-molecular-weight hyaluronic acid (high MW HA), the composition comprising a synthetic, isolated, or recombinant polypeptide comprising an amino acid sequence selected from: GPHGVREA (SEQ ID NO:1), GDGDGASA (SEQ ID NO: 2), GPMGPSGP (SEQ ID NO: 3), GLGPGARA (SEQ ID NO: 4), GPQGFQGP (SEQ ID NO: 5), GPMGPRGP (SEQ ID NO: 6), or GPGKNGDD (SEQ ID NO: 7), wherein the polypeptide comprises no more than 20 amino acids. In some embodiments, the amino acid sequence is GPHGVREA (SEQ ID NO:1). In some embodiments, the polypeptide is an octapeptide. In some embodiments, the polypeptide comprises octapeptide-45.

In some embodiments, the composition comprises a high molecular weight (MW) hyaluronic acid (HA) or derivatives thereof, wherein the high MW HA has a molecular weight of at least 0.5 MDa. In some embodiments, the high MW HA or derivatives thereof has a molecular weight of about 1 MDa to 4 MDa. In some embodiments, the high MW HA or derivatives comprises sodium hyaluronate. In some embodiments, the sodium hyaluronate comprise a sodium hyaluronate crosspolymer.

In some embodiments, the composition comprises a synthetic tripeptide. In some embodiments, the synthetic tripeptide is tetradecyl aminobutyroylvalylaminobutyric urea trifluoroacetate.

In some embodiments, composition comprises a hexapeptide. In some embodiments, the hexapeptide is hexapeptide-11. In some embodiments, the hexapeptide-11 is encapsulated in a liposome.

In some embodiments, the composition comprises lactoferrin. In some embodiments, the lactoferrin is encapsulated in a liposome.

In some embodiments, the composition comprises phosphatidylserine. In some embodiments, the composition comprises Tremella fuciformis extract. In some embodiments, the composition comprises hydroxymethoxyphenyl decanone. In some embodiments, the composition comprises a high molecular weight hyaluronic acid, synthetic tripeptide, a hexapeptide, lactoferrin, phosphatidylserine, Tremella fuciformis extract, or hydroxymethoxyphenyl decanone, or combinations thereof. In some embodiments, the composition comprises hexapeptide-12. In some embodiments, the composition comprises tripeptide-1. In some embodiments, the composition comprises tripeptide-1 and hexapeptide-12.

In some embodiments, the composition is aqueous.

In some embodiments, composition is effective to achieve a reduction in inflammation. IN some embodiments, the reduction in inflammation is assessed by a reduction or a lack of increase in an expression level of nitric oxide synthase 2 (NOS2), tumor necrosis factor (TNF), interleukin 12 (IL-12b), or cluster of differentiation-80 (CD80), or combinations thereof.

In some embodiments, the composition increases CD44 expression.

In some embodiments, the composition reduces redness of a skin of an individual after applying the composition. In some embodiments, the redness is measured by photography.

In some embodiments, the composition increases skin hydration in a skin of an individual after applying the composition. In some embodiments, the composition reduces or reverses signs of solar elastosis.

In some embodiments, the composition upregulates hyaluronic acid synthase (HAS) expression. In some embodiments, the HAS comprises HAS2.

In some embodiments, the composition downregulates hyaluronidase expression. In some embodiments, the hyaluronidase comprises hyaluronidase 2 (HYAL2).

In another aspect, which may be combined with any other aspect or embodiment, the present disclosure relates to a method for reducing inflammation in a skin of an individual, the method comprising: applying a topical composition comprising a synthetic, isolated, or recombinant polypeptide comprising an amino acid sequence GPHGVREA (SEQ ID NO:1), GDGDGASA (SEQ ID NO: 2), GPMGPSGP (SEQ ID NO: 3), GLGPGARA (SEQ ID NO: 4), GPQGFQGP (SEQ ID NO: 5), GPMGPRGP (SEQ ID NO: 6), or GPGKNGDD (SEQ ID NO: 7), wherein the polypeptide comprises no more than 20 amino acids. In some embodiments, the amino acid sequence is GPHGVREA (SEQ ID NO: 1). In some embodiments, the polypeptide is an octapeptide. In some embodiments, the polypeptide comprises octapeptide-45.

In some embodiments, the composition comprises a high molecular weight (MW) hyaluronic acid (HA) or derivatives thereof, wherein the high MW HA has a molecular weight of at least 0.5 MDa. In some embodiments, the high MW HA or derivatives thereof has a molecular weight of about 1 MDa to 4 MDa. In some embodiments, the high MW HA or derivatives comprises sodium hyaluronate. In some embodiments, the sodium hyaluronate comprise a sodium hyaluronate crosspolymer.

In some embodiments, the composition comprises a synthetic tripeptide. In some embodiments, the synthetic tripeptide is tetradecyl aminobutyroylvalylaminobutyric urea trifluoroacetate.

In some embodiments, the composition comprises a hexapeptide. In some embodiments, the hexapeptide is hexapeptide-11. In some embodiments, the hexapeptide-11 is encapsulated in a liposome.

In some embodiments, the composition comprises lactoferrin. In some embodiments, the lactoferrin is encapsulated in a liposome.

In some embodiments, the composition comprises phosphatidylserine. In some embodiments, the composition comprises Tremella fuciformis extract. In some embodiments, the composition comprises hydroxymethoxyphenyl decanone.

In some embodiments, the composition comprises a synthetic tripeptide, a hexapeptide, lactoferrin, phosphatidylserine, Tremella fuciformis extract, or hydroxymethoxyphenyl decanone, or combinations thereof. In some embodiments, the composition comprises hexapeptide-12. In some embodiments, the composition comprises tripeptide-1. In some embodiments, the composition comprises tripeptide-1 and hexapeptide-12.

In some embodiments, the composition is aqueous.

In some embodiments, the reduction in inflammation is assessed by a reduction or a lack of increase in an expression level of nitric oxide synthase 2 (NOS2), tumor necrosis factor (TNF), interleukin 12 (IL-12b), or cluster of differentiation-80 (CD80), or combinations thereof.

In some embodiments, the composition increases CD44 expression.

In some embodiments, the composition reduces redness of a skin of an individual after applying the composition. In some embodiments, the redness is measured by photography.

In some embodiments, the composition increases skin hydration in a skin of an individual after applying the composition. In some embodiments, the composition promotes HA synthesis.

In some embodiments, the composition upregulates hyaluronic acid synthase (HAS) expression. In some embodiments, the HAS comprises HAS2.

In some embodiments, the composition downregulates hyaluronidase expression. In some embodiments, the hyaluronidase comprises hyaluronidase 2 (HYAL2).

In some embodiments, the composition reduces appearance of a bruise, an aging spot, or a wrinkle.

In some embodiments, the composition is applied 1, 2, 3, 4, 5, 6, 7, or 8 times a day. In some embodiments, the individual is a human.

In another aspect, which may be combined with any other aspect or embodiment, the present disclosure relates to a topical composition for promoting skin repair, the topical composition comprising a synthetic, isolated, or recombinant octapeptide comprising an amino acid sequence Gly-X1-X2-X3-X₄-X5-X6-X7. In some embodiments, X1 is Pro, Asp, or Leu. In some embodiments, X2 is His, Gly, Met, or Gln. In some embodiments, X3 is Gly, Asp, Pro, or Lys. In some embodiments, X4 is Val, Gly, Pro, Phe, or Asn. In some embodiments, X5 is Arg, Ala, Ser, Gln, or Gly. In some embodiments, X6 is Glu, Ser, Gly, Arg, or Asp. In some embodiments, X7 is Ala, Pro, or Asp. In some embodiments, the octapeptides comprises an amino acid sequence feature found in collagen or elastin. In some embodiments, the amino acid sequence comprises GPHGVREA (SEQ ID NO:1), GDGDGASA (SEQ ID NO: 2), GPMGPSGP (SEQ ID NO: 3), GLGPGARA (SEQ ID NO: 4), GPQGFQGP (SEQ ID NO: 5), GPMGPRGP (SEQ ID NO: 6), GPGKNGDD (SEQ ID NO: 7), or GPMGPRGP (SEQ ID NO: 8). In some embodiments, the amino acid sequence is GPHGVREA (SEQ ID NO: 1). In some embodiments, the polypeptide comprises octapeptide-45.

In some embodiments, the composition comprises a high molecular weight (MW) hyaluronic acid (HA) or derivatives thereof, wherein the high MW HA has a molecular weight of at least 0.5 MDa. In some embodiments, the high MW HA or derivatives comprises sodium hyaluronate. In some embodiments, the sodium hyaluronate comprise a sodium hyaluronate crosspolymer.

In some embodiments, the composition comprises a synthetic tripeptide. In some embodiments, the synthetic tripeptide is tetradecyl aminobutyroylvalylaminobutyric urea trifluoroacetate.

In some embodiments, the composition comprises a hexapeptide. In some embodiments, the hexapeptide is hexapeptide-11. In some embodiments, the hexapeptide-11 is encapsulated in a liposome.

In some embodiments, the composition comprises lactoferrin. In some embodiments, the lactoferrin is encapsulated in a liposome.

In some embodiments, the composition comprises phosphatidylserine. In some embodiments, the composition comprises Tremella fuciformis extract. In some embodiments, the composition comprises hydroxymethoxyphenyl decanone.

In some embodiments, the composition comprises a synthetic tripeptide, an octapeptide, a hexapeptide, lactoferrin, phosphatidylserine, Tremella fuciformis extract, or hydroxymethoxyphenyl decanone, or combinations thereof. In some embodiments, the composition comprises hexapeptide-12. In some embodiments, the composition comprises tripeptide-1. In some embodiments, the composition comprises tripeptide-1 and hexapeptide-12.

In some embodiments, the composition is aqueous.

In some embodiments, the reduction in inflammation is assessed by a reduction or a lack of increase in an expression level of nitric oxide synthase 2 (NOS2), tumor necrosis factor (TNF), interleukin 12 (IL-12b), or cluster of differentiation-80 (CD80), or combinations thereof. In some embodiments, the composition increases CD44 expression.

In some embodiments, the composition reduces redness of a skin of an individual after applying the composition. In some embodiments, the redness is measured by photography.

In some embodiments, the composition increases skin hydration in a skin of an individual after applying the composition. In some embodiments, the composition promotes HA synthesis. In some embodiments, the composition promotes elastin production.

In some embodiments, the composition upregulates hyaluronic acid synthase (HAS) expression. In some embodiments, the HAS comprises HAS2. In some embodiments, the composition downregulates hyaluronidase expression. In some embodiments, the hyaluronidase comprises hyaluronidase 2 (HYAL2).

In some embodiments, the composition reduces or reverses a sign of solar elastosis.

In some embodiments, the composition stimulates synthesis of high MW HA having a MW of at least 0.5 MDa.

In another aspect, which may be combined with any other aspect or embodiment, the present disclosure relates to a topical composition for increasing the production of elastin in mammalian skin comprising: a polypeptide comprising an amino acid sequence selected from the group consisting of GPHGVREA (SEQ ID NO:1), GDGDGASA (SEQ ID NO: 2), GPMGPSGP (SEQ ID NO: 3), GLGPGARA (SEQ ID NO: 4), GPQGFQGP (SEQ ID NO: 5), GPMGPRGP (SEQ ID NO: 6), or GPGKNGDD (SEQ ID NO: 7), wherein the polypeptide comprises no more than 20 amino acids; tripeptide-1; and hexapeptide-12.

In some embodiments, the polypeptide is an octapeptide. In some embodiments, the amino acid sequence of the polypeptide comprises GPHGVREA (SEQ ID NO:1). In some embodiments, the octapeptide is octapeptide-45.

In some embodiments, the composition further comprises hexapeptide-11, tetradecylaminobutyroylvalylaminoburyric urea trifluoroacetate, lactoferrin, phosphatidylserine, Tremella fuciformis extract, hydroxymethoxyphenyl decanone, or any combination thereof. In some embodiments, the composition comprises a high molecular weight (MW) hyaluronic acid (HA) or derivatives thereof, wherein the high MW HA has a molecular weight of at least 0.5 MDa

In some embodiments, the composition comprises hexapeptide-11.

In some embodiments, the composition comprises lactoferrin.

In some embodiments, the composition comprises phosphatidylserine.

In some embodiments, the composition comprises Tremella fuciformis extract.

In some embodiments, the composition comprises tetradecylaminobutyroylvalylaminoburyric urea trifluoroacetate.

In some embodiments, the composition comprises hydroxymethoxyphenyl decanone.

In some embodiments, any one of the hexapeptide-11, tetradecylaminobutyroylvalylaminoburyric urea trifluoroacetate, lactoferrin, phosphatidylserine, Tremella fuciformis extract, hydroxymethoxyphenyl decanone, or any combination thereof, are encapsulated in a liposome.

In some embodiments, the composition is aqueous.

In some embodiments, the composition is effective to increase the production of tropoelastin and microfibrillar-associated protein 4 (MFAP4). In some embodiments, the composition is effective to upregulate expression of S100A2, LAMA3, MERTK, or ITGB4. In some embodiments, the composition upregulates one or more genes related to pathways related to ECM regulation.

In another aspect, which may be combined with any other aspect or embodiment, the present disclosure relates to a method of treating or restoring aging skin, the method comprising: administering to the skin a composition according to any of the embodiments described herein.

In another aspect, which may be combined with any other aspect or embodiment, the present disclosure relates to a method of upregulating expression of MFAP4, S100A2, LAMA3, MERTK, or ITGB4 expression in skin, the method comprising: administering to the skin a composition according to any of the embodiments described herein.

In another aspect, which may be combined with any other aspect or embodiment, the present disclosure relates to a method of increasing MFAP4 expression in skin, the method comprising: administering to the skin a composition according to any of the embodiments described herein.

In another aspect, which may be combined with any other aspect or embodiment, the present disclosure relates to a method of increasing S100A2 expression in skin, the method comprising: administering to the skin a composition according to any of the embodiments described herein.

In another aspect, which may be combined with any other aspect or embodiment, the present disclosure relates to a method of increasing LAMA3 expression in skin, the method comprising: administering to the skin a composition according to any of the embodiments described herein.

In another aspect, which may be combined with any other aspect or embodiment, the present disclosure relates to a method of increasing MERTK expression in skin, the method comprising: administering to the skin a composition according to any of the embodiments described herein.

In another aspect, which may be combined with any other aspect or embodiment, the present disclosure relates to a method of increasing ITGB4 expression in skin, the method comprising: administering to the skin a composition according to any of the embodiments described herein.

In another aspect, which may be combined with any other aspect or embodiment, the present disclosure relates to a method of upregulating genes related to pathways related to ECM regulation in skin, the method comprising: administering to the skin a composition according to any of the embodiments described herein.

In another aspect, which may be combined with any other aspect or embodiment, the present disclosure relates to a method of increasing MFAP4 expression in skin, the method comprising: administering to the skin a composition according to any of the embodiments described herein.

In another aspect, which may be combined with any other aspect or embodiment, the present disclosure relates to a method of increasing elastin production in human skin, the method comprising: administering to the skin a composition according to any of the embodiments described herein.

In another aspect, which may be combined with any other aspect or embodiment, the present disclosure relates to a composition according to any of the embodiments described herein, for use in treating or restoring aging skin or increasing elastin production in human skin.

In some embodiments, the composition increases expression of MFAP4, S100A2, LAMA3, MERTK, or ITGB4 in skin.

In some embodiments, the composition upregulates expression of genes related to pathways related to ECM regulation in skin.

In another aspect, which may be combined with any other aspect or embodiment, the present disclosure relates to a method for promoting wound healing, comprising administering to the skin the topical composition according to any of the embodiments described herein.

In another aspect, which may be combined with any other aspect or embodiment, the present disclosure relates to a method for promoting wound healing or wound closure, comprising administering to the skin the topical composition according to any of the embodiments described herein.

In another aspect, which may be combined with any other aspect or embodiment, the present disclosure relates to a method for treating a cutaneous wound, comprising administering to the skin the topical composition according to any of the embodiments described herein.

### BRIEF DESCRIPTION OF THE DRAWINGS

This patent application contains at least one drawing executed in color. Copies of this patent or patent application with color drawing(s) will be provided by the Office upon request and payment of the necessary fec.

FIG. 1 illustrates a graph of changes in fold of mRNA gene expression of Hyaluronic Acid Synthase (HAS2) in dermal fibroblasts following treatment with compounds listed below the bars compared with untreated cells.

FIG. 2A illustrates a graph of changes in fold of mRNA gene expression of Hyaluronic Acid Synthase (HAS2) in keratinocytes following treatment with compounds listed below the bars compared with untreated cells.

FIG. 2B illustrates a graph of changes in fold of mRNA gene expression of Hyaluronidase 2 (HYAL2) in keratinocytes following treatment with compounds listed below the bars compared with untreated cells.

FIG. 3 illustrates a graph of changes in fold of mRNA gene expression of Early Growth Response 3 (EGR3) in keratinocytes following treatment with compounds listed below the bars compared with untreated cells.

FIG. 4 depicts an SDS-PAGE gel with concentrated supernatant from cultured human dermal fibroblasts demonstrating the effects of treatment with various compounds.

FIG. 5 depicts an SDS-PAGE gel with concentrated supernatant from cultured human dermal fibroblasts demonstrating the effects of treatment with various compounds.

FIG. 6 illustrates a graph of dose response stimulation of high molecular weight HA production by Octapeptide treatment in dermal fibroblasts.

FIG. 7 is a line plot demonstrating a progressive increase in the mean hydration levels on clean skin 15 minutes post cleansing of facial skin.

FIG. 8 is a bar graph demonstrating a progressive increase in the mean hydration levels on clean skin following cleansing of facial skin, waiting a first 15-minute period post cleansing, applying the treatment with a composition comprising high MW HA and octapeptide and waiting a second 15-minute period post treatment prior to hydration measurement.

FIG. 9 is a table aggregating the results of participant assessment and satisfaction surveys following post cleansing the skin at various time points after the initiation of treatment with a composition comprising high MW HA and octapeptide.

FIG. 10 is a table aggregating the results of participant assessment surveys of their skin condition beginning at treatment initiation (Baseline) and at various time points after the initiation of treatment.

FIG. 11 is a table of participant satisfaction and a statistical analysis of the differences between baseline ratings and the ratings at various time points after the initiation of treatment.

FIG. 12 is a chart comparing mean investigator assessment scores for the given criteria of skin condition at baseline and following 8 weeks of treatment.

FIG. 13 is a chart comparing mean investigator assessment scores for skin moisture/hydration at baseline and following 8 weeks of treatment.

FIG. 14-21 are photographs, analysis and histology sections from subjects at baseline and at various time points following initiation of treatment with a composition comprising high MW HA and octapeptide, SPF 30+, an optional ultra-light moisturizer post Week 4, and regular skin care using a gentle cleanser.

FIG. 14A shows photographs taken at baseline and at Week 4 following treatment to demonstrate improvements to the skin in a female subject, age 39.

FIG. 14B shows analysis of photographs from FIG. 14A using the VISIA® Skin Analysis System to demonstrate a reduction in red areas of the face following treatment.

FIG. 15 shows photographs taken (from left to right) at baseline, at Week 2, at Week 4, and at Week 8 following treatment to demonstrate improvements to progressive effects of skin improvement in a female subject, age 35.

FIG. 16A shows close-up photographs taken at baseline and at Week 8 following treatment to demonstrate improvements in pore size, skin tone and skin texture in a female subject, age 35.

FIG. 16B shows analysis of photographs from FIG. 15 taken at baseline and at Week 8 following treatment using the VISIA® Skin Analysis System to demonstrate a reduction in red areas of the face.

FIG. 16C shows analysis of photographs from FIG. 15 taken at baseline and at Week 8 following treatment using the VISIA® Skin Analysis System to demonstrate a reduction in pore sizes on the face.

FIG. 16D shows close-up photographs of the eye and cheek area in a female subject, age 35 from FIG. 15 taken at baseline and at Week 8 following treatment to demonstrate improvements in fine lines, wrinkles, and redness reduction.

FIG. 16E shows analysis of photographs of the eye and cheek area in a female subject, age 35 from FIG. 15 taken at baseline and at Week 8 following treatment using the VISIA® Skin Analysis System to demonstrate a reduction wrinkle density, area, and distribution.

FIG. 17A shows photographs taken at baseline and at Week 8 following treatment to demonstrate improvements to the skin tone, texture and redness reduction in a female subject, age 38.

FIG. 17B shows analysis of photographs from FIG. 17A using the VISIA® Skin Analysis System to demonstrate an improvement to yield a more even skin texture in areas of the face following treatment.

FIG. 18A shows photographs taken (from left to right) at baseline, at Week 2, at Week 4, and at Week 8 following treatment to demonstrate improvements of a progressive reduction in skin inflammation, a reduction in skin redness, and a reduction in visibility of fine blood vessels in a male subject, age 48.

FIG. 18B shows close-up photographs of the upper left face from the male subject, age 48 from FIG. 18A taken at baseline and at Week 8 following treatment to demonstrate improvements rosacea and inflammation.

FIG. 18C shows close-up photographs of the right cheek from the male subject, age 48 from FIG. 18A taken at baseline and at Week 8 following treatment to demonstrate skin improvements including reduced visibility of fine blood vessels.

FIG. 19A shows close-up photographs of right cheek and mouth area of a female subject, age 73 taken at baseline and at Week 2 following treatment to demonstrate improvements in fine lines, deep wrinkles and skin crepiness.

FIG. 19B shows photographs of right side of the face of the female subject, age 73 from FIG. 19A taken at baseline and at Week 4 following treatment to demonstrate progressive improvements in fine lines, deep wrinkles and skin crepiness.

FIG. 19C shows photographs of right side of the face of the female subject, age 73 from FIG. 19A taken at baseline and at Week 8 following treatment to demonstrate progressive improvements in fine lines, deep wrinkles and skin crepiness.

FIG. 20A-C show histological sections of periauricular biopsies of subjects at baseline and at Week 8 following treatment to demonstrate marked improvement in solar elastotic extracellular matrix (ECM). Paired sections from three separate subjects at the given time points are stained with hematoxylin and eosin. Magnification is 200×.

FIG. 21A-C show histological sections of periauricular biopsies of subjects at baseline and at Week 8 following treatment to demonstrate marked upregulation of CD44 expression in epidermal and dermal structures in a third experiment. FIG. 21A has paired sections from a first subject; magnification at 200×. FIG. 21B-C have paired sections from a second subject; magnification at 200× and 400× respectively.

FIG. 22 shows a diagram of the chemical structure of octapeptide-45, comprising an amino acid sequence Gly-Pro-His-Gly-Val-Arg-Glu-Ala.

FIGS. 23A-23D are bar graphs showing results from in vitro gene expression studies (percent change versus untreated control) for human dermal fibroblasts cultured and treated with tripeptide-1+hexapeptide-12 (TriHex), octapeptide-45, and TriHex+octapeptide-45. FIG. 23A shows results for S100A2 expression; FIG. 23B shows results for LAMA3 expression; FIG. 23C shows results for MERTK expression; and FIG. 23D shows results for ITGB4 expression. All treatments were 20 μg/mL.

FIG. 24 is a schematic illustration of an ex vivo experimental model used for testing the effect on protein production for peptide compositions according to the present disclosure.

FIG. 25 is a schematic illustration of a model showing tropoelastin and MFAP4 production in elastin fiber assembly. (Reproduced from P. Bartosz et al., 291 J. Biol. Chem. 1103-14 (January 2016) (FIG. 10).)

FIG. 26 is an illustration of a human skin sample cross-section, indicating the location of the epidermis, papillary dermis, and reticular dermis.

FIG. 27 shows stained human skin sample cross-sections for untreated ski (left) and skin treated with tripeptide-1+hexapeptide-12 (TriHex) (second from left), octapeptide-45 (second from right), and TriHex+octapeptide-45 (right). The blue stain represents DAPI for nuclei and cells. The red stain represents MFAP4.

FIG. 28 shows stained human skin sample cross-sections for untreated ski (left) and skin treated with tripeptide-1+hexapeptide-12 (TriHex) (second from left), octapeptide-45 (second from right), and TriHex+octapeptide-45 (right). The blue stain represents DAPI for nuclei and cells. The red stain represents tropoelastin (TE).

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

Skin aging is associated with a loss of skin moisture and reduced barrier function. Aged or damaged skin can present with various signs of inflammation, which may be visible as increased skin redness, increased visibility of fine blood vessels, and certain skin conditions including, but not limited to, rosacea. Environmental influences, such as excessive UV light exposure, may contribute to the visible, progressive changes seen in aged skin.

Often, improving skin hydration and reducing inflammation in skin may help to treat damaged, inflamed, or aging skin. Application of topical compositions with peptides have been used to treat damaged, inflamed, or aging skin.

Described herein are compositions and methods comprising a topical formulation and application of an octapeptide. In some embodiments, the compositions and methods described herein reduce skin inflammation, appearance of solar elastosis, and/or signs of skin aging. In some embodiments, compositions and methods described herein improve skin health, appearance, and hydration. In some embodiments, the composition comprising an octapeptide results in increase synthesis of high MW HA as compared to a skin untreated with the composition. In some embodiments, the octapeptide in the composition stimulates synthesis of high MW HA. In some embodiments, the compositions increase elastin production.

As described herein, a group of active agents that stimulated high MW HA production in both epidermal and dermal cell types were identified. Members of one class of these active agents (e.g. octapeptides) comprise amino acid sequence features present in both collagen and elastin. Upon application of topical formulations comprising an octapeptide, endogenous signaling pathways may recognize these short peptide sequences that mimic extracellular matrix (ECM) degradation products and upregulate a natural response that produces high MW HA. These novel approaches to skin treatment described herein, including high MW HA plus peptide topical application, aim to increase production of exclusively high MW HA in both epidermal and dermal cells. These formulations of compositions and methods by which to use them were created to maximize hydration capacity in both the epidermis and dermis while encouraging regenerative activity within in the ECM. Application of an octapeptide formulation led to quantifiable increases in skin hydration; concurrent decreases in redness, inflammation, and the appearance of fine lines and wrinkles; and a striking reversal of solar elastotic ECM structure to a healthier and denser ECM network.

In some embodiments, the compositions comprising an octapeptide increase production of elastin in the skin. In some embodiments, the compositions comprise octapeptide-45, tripeptide-1, and hexapeptide-12. In some embodiments, the compositions upregulate expression of genes related to ECM regulation in the skin. In some embodiments, the compositions increase MFAP4 expression. In some embodiments, the compositions increase tropoelastin (TE) production. In some embodiments, the compositions upregulate SA1002 expression. In some embodiments, the compositions upregulate LAMA3 expression. In some embodiments, the compositions upregulate MERTK expression. In some embodiments, the compositions upregulate ITGB4 expression. In some embodiments, the compositions upregulate gene expression for one or more pathways related to ECM regulation.

### Compositions

### Peptides

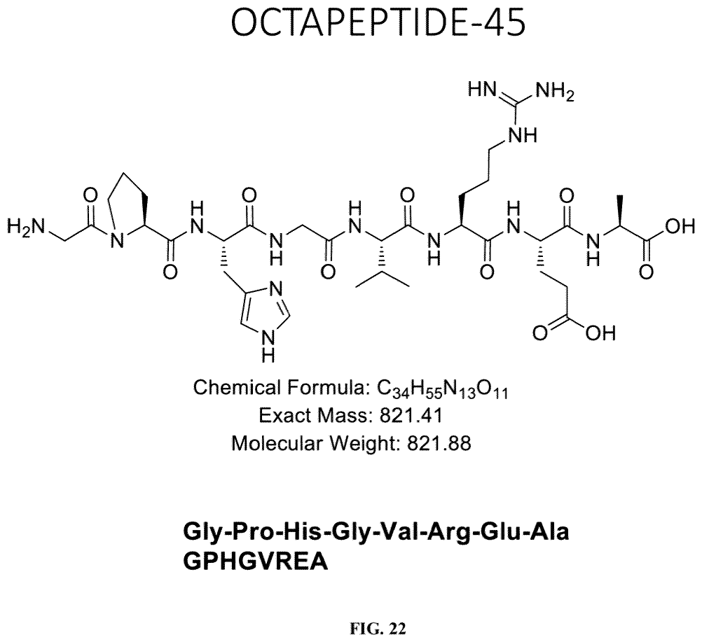

Described herein are compositions comprising an octapeptide and methods of preparing and using such compositions. In some embodiments, the compositions and methods comprise one or more peptides for inducing new elastogenesis in human skin or stimulating hyaluronic acid (HA) production. In some embodiments, the compositions and methods comprise one or more peptides for stimulating production of high molecular weight HA. In some embodiments, the octapeptide comprises octapeptide-45. In some embodiments, the octapeptide-45 is a synthetic octapeptide. In some embodiments, the octapeptide-45 is a synthetic octapeptide having the chemical structure illustrated in FIG. 22.

Compositions and methods described herein, in some embodiments, comprise one or more octapeptides. In some embodiments, the one or more octapeptides comprises an octapeptide amino acid sequence H-Gly-X₁-X₂-X₃-X₄-X₅-X₆-X₇-OH. In some embodiments, the octapeptide amino acid sequence comprises H-Gly-X₁-X₂-X₃-X₄-X₅-X₆-X₇-OH, wherein X₁ is Pro, Asp, or Leu. In some embodiments, the octapeptide amino acid sequence comprises H-Gly-X₁-X₂-X₃-X₄-X₅-X₆-X₇-OH, wherein X₂ is His, Gly, Met, or Gln. In some embodiments, the octapeptide amino acid sequence comprises H-Gly-X₁-X₂-X₃-X₄-X₅-X₆-X₇-OH, wherein X₃ is Gly, Asp, Pro, or Lys. In some embodiments, the octapeptide amino acid sequence comprises H-Gly-X₁-X₂-X₃-X₄-X₅-X₆-X₇-OH, wherein X₄ is Val, Gly, Pro, Phe, or Asn. In some embodiments, the octapeptide amino acid sequence comprises H-Gly-X₁-X₂-X₃-X₄-X₅-X₆-X₇-OH, wherein X₅ is Arg, Ala, Ser, Gln, or Gly. In some embodiments, the octapeptide amino acid sequence comprises H-Gly X₁-X₂-X₃-X₄-X₅-X₆-X₇-OH, wherein X₆ is Glu, Ser, Gly, Arg, or Asp. In some embodiments, the octapeptide amino acid sequence comprises H-Gly-X₁-X₂-X₃-X₄-X₅-X₆-X₇-OH, wherein X₇ is Ala, Pro, or Asp. In some embodiments, the octapeptide amino acid sequence comprises H-Gly-X₁-X₂-X₃-X₄-X₅-X₆-X₇-OH, wherein X₁ is Pro, Asp, or Leu; wherein X₂ is His, Gly, Met, or Gln; wherein X₃ is Gly, Asp, Pro, or Lys; wherein X₄ is Val, Gly, Pro, Phe, or Asn; wherein X₅ is Arg, Ala, Ser, Gln, or Gly; wherein X₆ is Glu, Ser, Gly, Arg, or Asp; and wherein X₇ is Ala, Pro, or Asp. In some embodiments, the octapeptide amino acid sequence comprises H-Gly-Pro-Met-Gly-Pro-X₅-Gly-Pro-OH, wherein X₅ is Ser or Arg. In some embodiments, the octapeptide amino acid sequence comprises one or more of GPHGVREA, GDGDGASA, GPMGPSGP, GLGPGARA, GPQGFQGP, GPMGPRGP, or GPGKNGDD. In some embodiments, the octapeptide is GPHGVREA. In some embodiments, the octapeptide has a chemical structure of octapeptide-45. In some embodiments, the octapeptide is octapeptide-45. In some embodiments, the octapeptide-45 is GPHGVREA.

In some embodiments, compositions and methods described herein comprise one or more octapeptides. In some embodiments, the one or more octapeptides comprise octapeptides with sequence features found in collagen and/or elastin. In some embodiments, the octapeptides with sequence features found in collagen and/or elastin comprise an amino acid sequence GXXXXXX. In some embodiments, the octapeptides with sequence features found in collagen and/or elastin comprise an amino acid sequence H-Gly-X₁-X₂-X₃-X₄-X₅-X₆-X₇-OH, wherein X₁ is Pro, Asp, or Leu; wherein X₂ is His, Gly, Met, or Gln; wherein X₃ is Gly, Asp, Pro, or Lys; wherein X₄ is Val, Gly, Pro, Phe, or Asn; wherein X₅ is Arg, Ala, Ser, Gln, or Gly; wherein X₆ is Glu, Ser, Gly, Arg, or Asp; and wherein X₇ is Ala, Pro, or Asp.

In some embodiments, the composition comprises an octapeptide, a hexapeptide, or a tripeptide, or a combination thereof. In some embodiments, the composition comprises a peptide with an amino acid sequence features found in collagen and/or elastin. In some embodiments, the composition comprises a peptide with an amino acid sequence features similar or identical to chemical or enzymatic degradative product of collagen. In some embodiments, the composition comprises a peptide with an amino acid sequence features similar or identical to chemical or enzymatic degradative product of elastin. In some embodiments, the compositions and methods comprise one or more peptides for inducing new elastogenesis in human skin or stimulating elastin production. In some embodiments, the compositions and methods comprise one or more peptides for inducing new elastogenesis in human skin or stimulating collagen production. In some embodiments, the compositions and methods comprise one or more peptides for inducing new elastogenesis in human skin or turnover of older, thin collagen fibers with thicker, healthier collagen fibers. In some embodiments, the compositions and methods comprise one or more peptides for inducing new elastogenesis in human skin or stimulating elastin and collagen production. In some embodiments, the one or more elastin-derived peptides is a hexapeptide. In some embodiments, the one or more elastin-derived peptides comprises an amino acid sequence XGVXXG. In some embodiments, the one or more elastin-derived peptides comprises a sequence according to amino acid sequence IGVAPG, VGVAPG, or VGVTAG. Elastin-derived peptides (e.g., XGVXXG) described herein, in some embodiments, are used for regeneration of the injured or aged human skin. In some embodiments, compositions and methods described herein comprise one or more tripeptides, one or more hexapeptides, one or more octapeptides, or combinations thereof. In some embodiments, the one or more peptides (e.g., elastin-derived peptide) is synthetic. In some embodiments, compositions and methods described herein comprise a mixture of peptides (e.g., an elastin-derived peptide) that provide synergistic effects (e.g., gene expression). In some embodiments, the compositions and methods comprising one or more peptides with amino acid sequence features found in collagen and/or elastin comprise an octapeptide.

In some embodiments, the peptide is an elastin-derived peptide. In some embodiments, the elastin-derived peptide comprises an amino acid sequence XGVXXG. In some embodiments, the elastin-derived peptide comprises a sequence according to amino acid sequence IGVAPG, VGVAPG, or VGVTAG. In some embodiments, compositions and methods described herein further comprise one or more tripeptides.

### Concentrations of Peptides

Compositions described herein, in some embodiments, comprise an octapeptide. In some embodiments, the octapeptide is provided at least or about 0.00001%, 0.00003%, 0.00005%, 0.0001%, 0.0003%, 0.0005%, 0.001%, 0.003%, 0.005%, 0.0055%, 0.01%, 0.03%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %), relative to the total weight of the composition. In some embodiments, the octapeptide is provided in a range of about 0.00001 wt. %-1.0 wt. %, 0.0001 wt. %-0.01 wt. %, about 0.0001 wt. % to 0.001 wt. %, about 0.0001 wt. % to 0.0005 wt. %, about 0.25% to about 10%, about 0.5% to about 8%, about 0.75% to about 6%, or about 1% to about 4% by weight. In some embodiments, the octapeptide is provided in a concentration of at least about 0.10, 0.25, 0.5, 0.75, 1, 2, 3, 4, 5, 6, 7, 8, 9, 10, 20, 30, 40, 50, 60, 70, 80, 90, 100, 110, 120, 130, 140, 150, 160, 170, 180, 190, or 200 parts per million (ppm). In some embodiments, the octapeptide is provided in a concentration of about 0.1 ppm to about 200 ppm, about 1 ppm to about 200 ppm, about 1 to about 150 ppm, about 1 to about 100 ppm, about 1 to about 100 ppm, about 1 to about 50 ppm, about 1 to about 25 ppm, about 1 to about 20 ppm, about 1 to about 10 ppm, or about 10 to about 100 ppm. In some embodiments, the octapeptide is provided in a concentration of about 10 ppm. In compositions, the octapeptide may be present in an amount of from about 50 ppm or less to about 100, 200, 300, 400, or 500 ppm or more, e.g., 50 ppm to 150 ppm. In compositions, the hexapeptide is typically present in an amount of from about 50 ppm or less to about 100, 200, 300, 400, or 500 ppm or more, e.g., 50 ppm to 150 ppm. In some embodiments, the octapeptide is provided in a concentration of at least about 0.25, 0.5, 0.75, 1, 2, 3, 4, 5, 6, 7, 8, 9, 10, 20, 30, 40, 50, 60, 70, 80, 90, 100, 110, 120, 130, 140, 150, 160, 170, 180, 190, or 200 μg/mL. In some embodiments, the octapeptide is provided in a concentration of about 1 to about 200 μg/mL, about 1 to about 150 μg/mL, about 1 to about 100 μg/mL, about 1 to about 50 μg/mL, about 1 to about 10 μg/mL, about 10 to about 100 μg/mL, about 10 to about 50 μg/mL, about 20 to about 100 μg/mL, or about 30 to about 100 μg/mL. In some embodiments, the octapeptide is provided in a concentration of about 10 μg/mL. In some embodiments, the octapeptide is provided in a concentration of about 10 μg/mL to 20 μg/mL. In some embodiments, the octapeptide is provided in a concentration of about 20 μg/mL. In some embodiments, the octapeptide is provided in a range of about 0.25 to about 10, about 0.5 to about 8, about 1 to about 6, or about 2 to about 4 microgram per milliliter.

Compositions as described herein comprise a varying concentration of peptide. In some instances, a peptide is present at about 1, 2, 3, 4, 5, 6, 7, 8, 9, 10, 15, 20, 25, 50, 75, 100, 200, 300, 400, 500, 600, 700, 800, 900, or 1000 ppm. In some instances, a peptide is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, a peptide is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (μg/mL). In some instances, a peptide is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, a peptide is present from about 0.01% to about 10%, about 0.01% to about 0.02%, about 0.01% to about 0.03%, about 0.01% to about 0.04%, about 0.01% to about 0.05%, about 0.01% to about 0.1%, about 1% to about 5%, or about 1% to about 10% by weight (wt. %).

Compositions as described herein, in some embodiments, comprise one or more peptides. In some instances, a peptide of the one or more peptides is present at about 50 ppm or less to 1000, 5000, 10000, 50000, 100000, 500000 ppm or more, e.g., 100 ppm of the peptide, or any other suitable amount. In some instances, a peptide of the one or more peptides is present at about 1, 2, 3, 4, 5, 6, 7, 8, 9, 10, 15, 20, 25, 50, 75, 100, 200, 300, 400, 500, 600, 700, 800, 900, 1000, or more than 1000 ppm. In some instances, a peptide of the one or more peptides is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, a peptide of the one or more peptides is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (μg/mL). In some instances, a peptide of the one or more peptides is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, a peptide of the one or more peptides is present from about 0.01% to about 10%, about 0.01% to about 0.02%, about 0.01% to about 0.03%, about 0.01% to about 0.04%, about 0.01% to about 0.05%, about 0.01% to about 0.1%, about 1% to about 5%, or about 1% to about 10% by weight (wt. %). In some embodiments, a peptide of the one or more peptides is provided at least or about 0.00001%, 0.00003%, 0.00005%, 0.0001%, 0.0003%, 0.0005%, 0.001%, 0.003%, 0.005%, 0.0055%, 0.01%, 0.03%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, a peptide of the one or more peptides is provided in a range of about 0.25% to about 10%, about 0.5% to about 8%, about 0.75% to about 6%, or about 1% to about 4% by weight. In some embodiments, each peptide of the one or more peptides is provided in a range of about 0.001% to about 6%, about 0.002% to about 4%, about 0.01% to about 3%, or about 0.02% to about 2% by weight.

In some embodiments, the tripeptide is a synthetic tripeptide. In some embodiments, the synthetic tripeptide is tetradecyl aminobutyroylvalylaminobutyric urea trifluoroacetate. In compositions, the tripeptide is typically present in an amount of from about 50 ppm or less to about 100, 200, 300, 400, or 500 ppm or more, e.g., 50 ppm to 150 ppm. In compositions, the hexapeptide is typically present in an amount of from about 50 ppm or less to about 100, 200, 300, 400, or 500 ppm or more, e.g., 50 ppm to 150 ppm.

In some embodiments, the tripeptide is tripeptide-1. In some embodiments, the tripeptide-1 is provided at least or about 0.00001%, 0.00003%, 0.00005%, 0.0001%, 0.0003%, 0.0005%, 0.001%, 0.003%, 0.005%, 0.0055%, 0.01%, 0.03%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the tripeptide-1 is provided in a range of about 0.01 wt. % to about 2 wt. %, about 0.25% to about 10%, about 0.5% to about 8%, about 0.75% to about 6%, or about 1% to about 4% by weight. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the tripeptide-1 is provided in a range of about 0.25 to about 10, about 0.5 to about 8, about 1 to about 6, or about 2 to about 4 ppm. In some embodiments, the tripeptide-1 is provided in a range of about 1 to about 10 ppm. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (μg/mL). In some embodiments, the tripeptide-1 is provided in a range of about 0.25 to about 10, about 0.5 to about 8, about 1 to about 6, or about 2 to about 4 microgram per milliliter.

In some embodiments, the elastin-derived peptide is a synthetic peptide. In some embodiments, the elastin-derived peptide is a peptide that comprises elastogenic potential that mimics the elastin-derived VGVAPG, IGVAPG, VGVTAG (identified in the IGF-1-binding protein-1 (IGFBP-1)), or derivatives thereof. In some embodiments, the elastin-derived peptide is a synthetic peptide that comprises elastogenic potential that mimics the elastin-derived VGVAPG, IGVAPG, VGVTAG (identified in the IGF-1-binding protein-1 (IGFBP-1)), or derivatives thereof. In some embodiments, the elastin-derived peptide comprises an amino acid sequence XGVXXG. In some embodiments, the elastin-derived peptide comprises a sequence according to amino acid sequence IGVAPG, VGVAPG, or VGVTAG. In some embodiments, the synthetic peptide is a hexapeptide. In some embodiments, the hexapeptide is hexapeptide-11. In some embodiments, hexapeptide-11 is encapsulated in a liposome. In some embodiments, tripeptide is encapsulated in a liposome. In some embodiments, octapeptide is encapsulated in a liposome. In some embodiments, octapeptide-45 is encapsulated in a liposome. In some embodiments, TriHex and octapeptide-45 are encapsulated in a liposome. In some embodiments, the synthetic peptide is a lactoferrin. In some embodiments, lactoferrin is encapsulated in a liposome. In some embodiments, encapsulation in a liposome increases the efficiency of peptide delivery to cells. In some embodiments, encapsulation in a liposome increases the efficiency of peptide delivery to cells in deeper layers of the skin. In some embodiments, encapsulation in a liposome increases the efficiency of peptide delivery to cells in deeper epidermal layers of the skin. In some embodiments, encapsulation in a liposome increases the efficiency of peptide delivery to cells in deeper dermal layers of the skin. In some embodiments, encapsulation in a liposome increases half-life of a peptide in a formulation. In some embodiments, encapsulation in a liposome increases half-life of a tripeptide in a formulation. In some embodiments, encapsulation in a liposome increases half-life of a hexapeptide-11 in a formulation. In some embodiments, encapsulation in a liposome increases half-life of an octapeptide in a formulation. In some embodiments, encapsulation in a liposome increases half-life of octapeptide-45 in a formulation. In some embodiments, encapsulation in a liposome yields a formulation having a sustained release of a peptide upon application. In some embodiments, encapsulation in a liposome yields a formulation having a sustained release of a peptide upon topical application. In some embodiments, encapsulation in a liposome yields a formulation having a sustained release of a peptide upon topical application to facial skin.

In some embodiments, the elastin-derived peptide is provided at least or about 0.00001%, 0.00003%, 0.00005%, 0.0001%, 0.0003%, 0.0005%, 0.001%, 0.003%, 0.005%, 0.0055%, 0.01%, 0.03%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the elastin-derived peptide is provided in a range of about 0.00001% to about 10%, about 0.0001% to about 0.01%, about 0.0003% to about 9%, about 0.0005% to about 8%, about 0.001% to about 4%, or about 0.001% to 2% by weight (wt. %), or any range or value therein between. In some embodiments, the elastin-derived peptide is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the elastin-derived peptide is provided in a range of about 1 to about 10 ppm. In some embodiments, the elastin-derived peptide is provided in a range of about 0.25 to about 10, about 0.5 to about 8, about 1 to about 6, or about 2 to about 4 ppm. In some embodiments, the elastin-derived peptide is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (μg/mL). In some embodiments, the elastin-derived peptide is provided in a range of about 0.25 to about 10, about 0.5 to about 8, about 1 to about 6, or about 2 to about 4 microgram per milliliter.

In some embodiments, the hexapeptide-12 (VGVAPG) is provided at least or about 0.00001%, 0.00003%, 0.00005%, 0.0001%, 0.0003%, 0.0005%, 0.001%, 0.003%, 0.005%, 0.0055%, 0.01%, 0.03%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the hexapeptide-12 is provided in a range of about 0.00001% to about 10%, about 0.0001% to about 0.01%, about 0.0003%, to about 9%, about 0.0005% to about 8%, or about 0.001% to about 4%, about 0.001% to about 2% by weight (wt. %), or any range or value therein between. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the hexapeptide-12 is provided in a range of about 1 to about 10 ppm. In some embodiments, the hexapeptide-12 is provided in a range of about 0.25 to about 10, about 0.5 to about 8, about 1 to about 6, or about 2 to about 4 ppm. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (μg/mL). In some embodiments, the hexapeptide-12 is provided in a range of about 0.25 to about 10, about 0.5 to about 8, about 1 to about 6, or about 2 to about 4 microgram per milliliter.

Compositions described herein, in some embodiments, comprise hexapeptide-11. In some embodiments, the hexapeptide-11 is provided at least or about 0.00001%, 0.00003%, 0.00005%, 0.0001%, 0.0003%, 0.0005%, 0.001%, 0.003%, 0.005%, 0.0055%, 0.01%, 0.03%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the hexapeptide-11 is provided in a range of about 0.00001% to about 10%, about 0.0001% to about 0.01%, about 0.0003% to about 8%, about 0.0005%, to about 6%, about 0.001% to about 4%, about 0.001% to about 2%, about 0.005% to about 2%, or about 0.01% to about 1% by weight (wt. %), or any range or value therein between. In some embodiments, the hexapeptide-11 is provided in a range of about 0.001% to about 6%, about 0.002% to about 4%, about 0.01% to about 3%, or about 0.02% to about 2%. In some embodiments, the hexapeptide-11 is provided in a range of about 0.005% to about 0.02% by weight. In some embodiments, the hexapeptide-11 is provided at least or about 0.1 ppm, 3 ppm, 5 ppm, 10 ppm, 50 ppm, 55 ppm, 500 ppm, 1,000 ppm, 2,500 ppm, 5,000 ppm, or more than 5,000 ppm. In some embodiments, the hexapeptide-11 is provided in a range of about 5 ppm to about 100 ppm, about 10 ppm to about 1000 ppm, about 50 ppm to about 1500 ppm, or about 500 ppm to about 5,000 ppm. In some embodiments, the hexapeptide-11 is about 1000 ppm. In some embodiments, the hexapeptide-11 is provided at least or about 5, 10, 20, 25, 50, 75, 100, 150, 200, 250, 300, 350, 400, 450, 500, or more than 500 microgram per milliliter (μg/mL). In some embodiments, the hexapeptide-11 is provided in a range of about 25 to about 250, about 50 to about 200, about 75 to about 150, about 200 to about 300, or about 200 to about 400 microgram per milliliter.

### Modification of Peptides

The peptide can be functionalized. For example, the peptide can be functionalized with a fatty acid, e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, or the like. Examples include palmitoyl hexapeptide-12 (Pal-VGVAPG), palmitoyl tripeptide-1 (Pal-GHK), myristoyl hexapeptide-12 (Myr-VGVAPG), and myristoyl tripeptide-1 (Myr-GHK). Palmitoyl or myristoyl functionalization can be desirable in certain embodiments as it exhibits enhanced penetration when compared to other fatty acids. In some embodiments, the peptide is functionalized with a chemical group. For example, the peptide is functionalized with acetyl. In some instances, the peptide is functionalized with a functional group comprising no more than 14 carbons. In some instances, the peptide is functionalized with a functional group comprising no more than 3, 4, 5, 6, 7, 8, 9, 10, 11, 12, 13, 14, 15, 16, 17, 18, 19, 20, or more than 20 carbons. In some instances, the peptide is non-palmitoylated. Without wishing to be limited to a particular theory, incorporation of the peptide in a liposome, in some embodiments, increases the lipophilicity of a peptide that is functionalized or is not functionalized.

Some embodiments of the methods and compositions provided herein include as a first peptide glycine-histidine-lysine (GHK). GHK is a peptide sequence that is rarely found in the class of proteins in general, but is frequently found in extracellular matrix proteins. The small size of GHK permits it to approach membrane receptors far more easily than larger peptides. Further, its unique, copper-binding structure enhances copper transport into and out of cells and promotes wound healing through several different but related pathways. Due to its strong copper binding structure, GHK can be provided in the form of GHK-Cu (copper-bound GHK form).

In some embodiments, compositions described herein comprise an octapeptide. In some embodiments, the octapeptide comprises an amino acid sequence GDGDGASA (SEQ ID NO: 1), GPMGPSGP (SEQ ID NO: 2), GLGPGARA (SEQ ID NO: 3), GPQGFQGP (SEQ ID NO: 4), GPHGVREA (SEQ ID NO: 5), GPMGPRGP (SEQ ID NO: 6), GPGKNGDD (SEQ ID NO: 7), or GPMGPRGP (SEQ ID NO: 8). In some embodiments, the octapeptide comprises an amino acid sequence GDGDGASA (SEQ ID NO: 1). In some embodiments, the octapeptide comprises an amino acid sequence GPMGPSGP (SEQ ID NO: 2). In some embodiments, the octapeptide comprises an amino acid sequence GLGPGARA (SEQ ID NO: 3). In some embodiments, the octapeptide comprises an amino acid sequence GPQGFQGP (SEQ ID NO: 4). In some embodiments, the octapeptide comprises an amino acid sequence GPHGVREA (SEQ ID NO: 5). In some embodiments, the octapeptide comprises an amino acid sequence GPMGPRGP (SEQ ID NO: 6). In some embodiments, the octapeptide comprises an amino acid sequence GPGKNGDD (SEQ ID NO: 7). In some embodiments, the octapeptide comprises an amino acid sequence GPMGPRGP (SEQ ID NO: 8). In some embodiments, octapeptide-45 is GPHGVREA (SEQ ID NO: 5).

In some embodiments, the octapeptide is a synthetic peptide. In some embodiments, the octapeptide is a peptide that stimulates HA synthesis. In some embodiments, the octapeptide is a peptide that stimulates high molecular weight hyaluronic acid (high MW HA) synthesis. In some embodiments, the octapeptide is a peptide that stimulates high MW HA synthesis in the dermis. In some embodiments, the octapeptide is a peptide that stimulates high MW HA synthesis in the skin. In some embodiments, the octapeptide comprises an octapeptide having an amino acid sequence GPHGVREA, GDGDGASA, GPMGPSGP, GLGPGARA, GPQGFQGP, GPMGPRGP, or GPGKNGDD. In some embodiments, the octapeptide comprises GPHGVREA. In some embodiments, the octapeptide is GPHGVREA. In some embodiments, the octapeptide comprises a chemical structure of octapeptide-45 (FIG. 22).

### Liposomes

In some embodiments, compositions according to the present disclosure comprise liposomes for improved distribution, efficacy, bioavailability, and/or activity. Liposomal compositions may improve distribution, efficacy, bioavailability, and/or activity of the active ingredient by improving delivery and tissue (e.g., skin) penetration. In some instances, improved delivery and skin penetration result from the active ingredient being incorporated (e.g., encapsulated) in a liposome. In some instances, the active ingredient is one or more peptides encapsulated in a liposome.

Liposomal compositions as described herein may comprise a peptide encapsulated in a liposome. In some embodiments, the peptide encapsulated in the liposome is selected from the group consisting of hexapeptide-12, tripeptide-1, one or more octapeptides (e.g., octapeptide-45), hexapeptide-11, hexapeptide-38, tetrapeptide-2, or any combination thereof. In some embodiments, the peptide or peptides encapsulated in a liposome are functionalized with an acetyl group, a palmitoyl group, a myristoyl group, or any combination thereof.

Liposomal compositions as described herein may comprise various ingredients encapsulated in a liposome. In some embodiments, the ingredient is lactoferrin. In some embodiments, the ingredient is phosphatidylserine. In some embodiments, the ingredient is Ledum palustre extract. In some embodiments, the ingredient is Arnica montana extract. In some embodiments, the ingredient is sodium hyaluronate. In some embodiments, the ingredient is larger than 50 kDa.

Lecithin and other phospholipids may be used to prepare liposomes containing the peptide compositions as described herein. In some embodiments, liposomes are used to prepare one or more peptides. In some embodiments, the peptide is functionalized with an acetyl group. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the peptide compositions as described herein.

The phospholipids used to prepare the liposomal compositions described herein may comprise a transition phase temperature of about 10° C. to about 25° C. In some instances, the phospholipids comprise a transition phase temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the phospholipids comprise a transition phase temperature in a range of about 10° C. to about 40° C., about 12° C. to about 36° C., about 14° C. to about 32° C., about 16° C. to about 20° C., or about 21° C. to about 25° C.

The topical composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting composition contains micelles, i.e., spherical oil droplets.

The liposomal composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Described herein, in some embodiments, are methods for preparing a composition comprising a peptide encapsulated in a liposome, comprising: combining the peptide and a solvent to form a mixture; and contacting the mixture with an aqueous solution comprising liposomes. In some instances, the contacting occurs at a temperature between about 10° C. and about 25° C. In some instances, the contacting occurs at a temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the contacting occurs at a temperature in a range of about 10° C. to about 40° C., about 12° C. to about 36° C., about 14° C. to about 32° C., about 16° C. to about 20° C., or about 21° C. to about 25° C.

Methods for preparing a composition comprising a peptide encapsulated in a liposome may comprise use of a solvent. In some instances, the solvent is water. In some instances, the solvent is an organic solvent. Exemplary organic solvents include, but are not limited to, petroleum ether, cyclohexane, toluene, carbon tetrachloride, dichloromethane, chloroform, diethyl ether, diisopropyl ether, ethyl acetate, butanol, n-propanol, ethanol, methanol, polyethylene glycol, propylene glycol, and pyridine. In some instances, the solvent is a glycol. In some instances, the solvent is butylene glycol. In some instances, the solvent is caprylyl glycol. In some instances, the solvent is propanediol (propylene glycol).

The solvent may be used at various percentages. In some instances, the solvent is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10%. The solvent may be propanediol, butylene glycol, or caprylyl glycol.

Methods as described herein, in some embodiments, comprises combining the peptide and a solvent to form a mixture; and contacting the mixture with an aqueous solution comprising liposomes, wherein the aqueous solution comprises a percentage of water and a percentage of liposomes. In some instances, the aqueous solution comprises at least or about 20%, 30%, 40%, 50%, 60%, 70%, 80%, 90%, or more than 90% water. In some instances, the aqueous solution comprises water in a range of about 10% to about 95%, about 20% to about 90%, about 30% to about 85%, about 40% to about 80%, or about 50% to about 60%. In some instances, the aqueous solution comprises at least or about 20%, 30%, 40%, 50%, 60%, or more than 60% liposomes. In some instances, the aqueous solution comprises liposomes in a range of about 10% to about 80%, about 20% to about 70%, or about 30% to about 60%. A ratio of liposomes to water may be in a range of about 1:9 to about 3:7. In some instances, the ratio of liposomes to water may be at least or about 1:10, 1:9, 1:8, 1:7, 1:6, 1:5, 1:4, 1:3, or 1:2.

Methods for generation of liposomal compositions as described herein may result in an entrapment efficacy of no more than 100%. In some instances, the entrapment efficacy is no more than 50%, 60%, 70%, 80%, 90%, 95%, 99%, or 99.5%.

Described herein are liposomal compositions, wherein the peptide or peptides comprise(s) a percentage of the composition. In some embodiments, the peptide or peptides are provided at least or about 0.00001%, 0.00003%, 0.00005%, 0.0001%, 0.0003%, 0.0005%, 0.001%, 0.003%, 0.005%, 0.0055%, 0.01%, 0.03%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% of the composition. In some embodiments, the peptide or peptides are provided at least or about 10%, 11%, 12%, 13%, 14%, 15%, 16%, 17%, 18%, 19%, 20%, 22%, 24%, 26%, 28%, 30% or more than 30% of the composition. In some embodiments, the peptide or peptides are provided in a range of about 0.001% to about 6%, about 0.002% to about 4%, about 0.01% to about 5%, or about 0.02% to about 2% by weight. In some embodiments, the peptide is provided at about 0.03% of the composition.

Described herein are liposomal compositions, wherein the liposomes comprise a percentage of the composition. In some embodiments, the liposomes are provided at least or about 10%, 11%, 12%, 13%, 14%, 15%, 16%, 17%, 18%, 19%, 20%, 22%, 24%, 26%, 28%, 30% or more than 30% of the composition. In some embodiments, the liposomes are provided in a range of about 5% to about 90%, about 10% to about 80%, about 20% to about 70%, about 30% to about 60%, about 10% to about 30%, or about 20% to about 40%. In some embodiments, the liposomes are provided at about 30%. In some embodiments, the liposomes are provided at 27%.

In some embodiments, liposomal compositions as described herein comprise an average particle size of at most 220 nanometers (nm). In some instances, the average particle size is at most 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is about 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is in a range of about 50 nm to about 500 nm, about 100 nm to about 400 nm, about 150 nm to about 220 nm, about 180 nm to about 220 nm, or about 190 nm to about 210 nm.

In some instances, the liposomal compositions comprise an active agent that has a molecular weight of no more than about 600 Daltons (Da). In some instances, the active agent has a molecular weight of at least or about 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, or more than 1000 Daltons (Da). In some instances, the active agent has a molecular weight of at least or about 1000, 1100, 1200, 1300, 1400, 1500, 1600, 1700, 1800, 1900, 2000, 2100, 2200, 2300, 2400, 2500, 2600, 2700, 2800, 2900, 3000, 4000, 5000, 6000, or more than 6000 Daltons (Da). In some instances, the active agent has a molecular weight in a range of about 50 to about 1000, about 100 to about 900, about 200 to about 800, about 300 to about 700, or about 400 to about 600 Daltons (Da). In some instances, the active agent is a peptide or peptides as disclosed herein. In some instances, the active agent is a peptide or peptides encapsulated in a liposome.

A polydispersity index (PdI) of a liposomal composition as described herein, in some embodiments, is in a range of 0 to about 0.2. In some instances, the polydispersity index is about 0.01, 0.025, 0.05, 0.1, 0.25, 0.3, 0.35, 0.4, 0.45, 0.5, 0.55, 0.6, 0.65, 0.7, 0.75, or 0.8. In some instances, the polydispersity index is in a range of about 0.01 to about 0.8, about 0.025 to about 0.75, about 0.05 to about 0.6, or about 0.1 to about 0.3.

In some instances, an intercept of a liposomal composition as described herein is in a range of about 0.85 to about 0.95. In some instances, the intercept is the amplitude. In some instances, the intercept is at least or about 0.65, 0.70, 0.75, 0.80, 0.85, 0.90, or 0.95.

In some embodiments, the liposomes comprise propanediol, lecithin, or a combination thereof. In some embodiments, the propanediol is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the propanediol is provided in a range of about 0.001% to about 6%, about 0.002% to about 4%, about 0.01% to about 3%, or about 0.02% to about 2% by weight. In some embodiments, the lecithin is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %), relative to the total weight of the composition. In some embodiments, the lecithin is provided in a range of about 0.001% to about 6%, about 0.002% to about 4%, about 0.01% to about 3%, or about 0.02% to about 2% by weight, relative to the total weight of the composition. In some embodiments, the liposomes comprise propanediol and lecithin. In some embodiments, the propanediol and lecithin are provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %), relative to the total weight of the composition. In some embodiments, the propanediol and lecithin are provided in a range of about 0.001% to about 6%, about 0.002% to about 4%, about 0.01% to about 3%, or about 0.02% to about 2% by weight, relative to the total weight of the composition. In some embodiments, the propanediol and lecithin are provided at about 0.90% by weight, relative to the total weight of the composition.

Described herein are liposomal compositions comprising improved distribution, efficacy, bioavailability, and/or activity. The liposomal compositions may comprise improved distribution, efficacy, bioavailability, and/or activity as compared to compositions not comprising liposomes. In some instances, the distribution is improved by at least or about 0.5×, 1.0×, 1.5×, 2.0×, 2.5×, 3.0×, 4.0×, 4.5×, 5×, or more than 5× as compared to compositions not comprising liposomes. In some instances, the efficacy is improved by at least or about 0.5×, 1.0×, 1.5×, 2.0×, 2.5×, 3.0×, 4.0×, 4.5×, 5×, or more than 5× as compared to compositions not comprising liposomes. In some instances, the bioavailability is improved by at least or about 0.5×, 1.0×, 1.5×, 2.0×, 2.5×, 3.0×, 4.0×, 4.5×, 5×, or more than 5× as compared to compositions not comprising liposomes. In some instances, the activity is improved by at least or about 0.5×, 1.0×, 1.5×, 2.0×, 2.5×, 3.0×, 4.0×, 4.5×, 5×, or more than 5× as compared to compositions not comprising liposomes. The distribution, efficacy, bioavailability, and/or activity may be improved by at least or about 10%, 15%, 20%, 25%, 30%, 35%, 40%, 45%, 50%, 55%, 60%, 65%, 70%, 75%, 80%, 85%, 90%, or more than 90% as compared to compositions not comprising liposomes.

Liposomal compositions and methods as described herein, in some embodiments, are topical compositions. In some instances, the liposomal compositions are oil free. In some instances, the liposomal compositions are preservative free. In some embodiments, the liposomal formulation is an aqueous formulation. In some embodiments, the liposomal formulation is an anhydrous formulation. In some instances, the liposomal composition comprises a pH in a range of about 5 to about 8. In some instances, the liposomal composition comprises a pH of at least or about 2, 3, 4, 5, 6, 7, 8, 9, or 10.

Methods and compositions as described herein may result in improved follicular penetration. In some instances, the follicular penetration is improved by at least or about 0.5×, 1.0×, 1.5×, 2.0×, 2.5×, 3.0×, 4.0×, 4.5×, 5×, or more than 5×. The follicular penetration may be improved by at least or about 10%, 15%, 20%, 25%, 30%, 35%, 40%, 45%, 50%, 55%, 60%, 65%, 70%, 75%, 80%, 85%, 90%, or more than 90%. In some instances, compositions result in follicular penetration of a depth of at least or about 0.5, 0.75, 1, 1.25, 1.5, 2, 2.5, 3, 3.5, 4, 4.5, 5, 5.5, 6, 7, 8, 9, 10, or more than 10 millimeters.

### Gene Expression

Compositions described herein, in some embodiments, alter gene expression in skin cells. In some embodiments, exposure of dermal cells (e.g., dermal fibroblasts) to an octapeptide results in upregulation of HAS2 expression within dermal cells. In some embodiments, the dermal cells comprise dermal fibroblasts. In some embodiments, the octapeptide comprises one or more octapeptides with the amino acid sequence GPHGVREA, GDGDGASA, GPMGPSGP, GLGPGARA, GPQGFQGP, GPMGPRGP, or GPGKNGDD. In some embodiments, the octapeptide is octapeptide-45.

In some embodiments, exposure of dermal cells (e.g., dermal fibroblasts) or epidermal cells (e.g., keratinocytes) to a hexapeptide (e.g., hexapeptide-11) results in upregulation of HAS2 expression. In some embodiments, exposure of dermal cells (e.g., dermal fibroblasts) or epidermal cells (e.g., keratinocytes) to a tripeptide and a hexapeptide (TriHex-hexapeptide-12+tripeptide-1) results in upregulation of HAS2 expression. In some embodiments, exposure of epidermal cells (e.g., keratinocytes) to a hexapeptide (e.g., hexapeptide-11) results in upregulation of HAS2 expression. In some embodiments, exposure of epidermal keratinocytes to TriHex results in upregulation of HAS2 expression. In some embodiments, exposure of epidermal keratinocytes to hexapeptide-11 results in upregulation of HAS2 expression.

In some embodiments, exposure of epidermal cells (e.g., keratinocytes) to a hexapeptide (e.g., hexapeptide-11) results in downregulation of HYAL2 expression. In some embodiments, exposure of epidermal keratinocytes to hexapeptide-11 results in downregulation of HYAL2 expression.

In some embodiments, exposure of epidermal cells to a hexapeptide or octapeptide results in upregulation of EGR3 expression. In some embodiments, exposure of epidermal (e.g., keratinocytes) cells to tripeptide-1 and hexapeptide-12 (TriHex) results in upregulation of EGR3 expression. In some embodiments, exposure of epidermal cells to hexapeptide-11 results in upregulation of EGR3 expression. In some embodiments, exposure of epidermal keratinocytes to hexapeptide results in upregulation of EGR3 expression. In some embodiments, exposure of epidermal keratinocytes to hexapeptide-11 results in upregulation of EGR3 expression. In some embodiments, exposure of epidermal keratinocytes to an octapeptide (e.g., octapeptide-45) results in upregulation of EGR3 expression.

In some embodiments, exposure of human dermal fibroblasts to TriHex results in upregulation of LAMA3 expression. In some embodiments, exposure of human dermal fibroblasts to TriHex results in upregulation of MERTK expression. In some embodiments, exposure of human dermal fibroblasts to an octapeptide (e.g., octapeptide-45) results in upregulation of S100A2 expression. In some embodiments, exposure of human dermal fibroblasts to an octapeptide (e.g., octapeptide-45) results in upregulation of LAMA3 expression. In some embodiments, exposure of human dermal fibroblasts to an octapeptide (e.g., octapeptide-45) results in upregulation of MERTK expression. In some embodiments, exposure of human dermal fibroblasts to an octapeptide (e.g., octapeptide-45) results in upregulation of ITGB4 expression.

In some embodiments, exposure of human dermal fibroblasts to TriHex+octapeptide (e.g., octapeptide-45) results in upregulation of S100A2 expression. In some embodiments, exposure of human dermal fibroblasts to TriHex+octapeptide (e.g., octapeptide-45) results in synergistic upregulation of S100A2 expression, when compared to expression after exposure to TriHex alone or octapeptide alone. In some embodiments, exposure of human dermal fibroblasts to TriHex+octapeptide (e.g., octapeptide-45) results in upregulation of LAMA3 expression. In some embodiments, exposure of human dermal fibroblasts to TriHex+octapeptide (e.g., octapeptide-45) results in synergistic upregulation of LAMA3 expression, when compared to expression after exposure to TriHex alone or octapeptide alone. In some embodiments, exposure of human dermal fibroblasts to TriHex+octapeptide (e.g., octapeptide-45) results in upregulation of MERTK expression. In some embodiments, exposure of human dermal fibroblasts to TriHex+octapeptide (e.g., octapeptide-45) results in synergistic upregulation of MERTK expression, when compared to expression after exposure to TriHex alone or octapeptide alone. In some embodiments, exposure of human dermal fibroblasts to TriHex+octapeptide (e.g., octapeptide-45) results in upregulation of ITGB4 expression. In some embodiments, exposure of human dermal fibroblasts to TriHex+octapeptide (e.g., octapeptide-45) results in synergistic upregulation of ITGB4 expression, when compared to expression after exposure to TriHex alone or octapeptide alone.

### Hyaluronic Acid Synthesis

In some embodiments, the composition comprising an octapeptide results in increase synthesis of high MW HA as compared to a skin untreated with the composition. In some embodiments, the octapeptide in the composition stimulates synthesis of high MW HA. In some embodiments, the increase in HA synthesis is by stimulation of hyaluronic acid synthase (HAS) activity. In some embodiments, the HAS comprises HAS2. In some embodiments, the increase in HA synthesis is by downregulation of hyaluronidase expression. In some embodiments, the hyaluronidase comprises hyaluronidase 2 (HYAL2). In some embodiments, the octapeptide stimulates HAS2 in fibroblasts, enzyme that stimulates HA production. In some embodiments, the octapeptide is encapsulated into a phospholipid delivery system for increased absorption and activity. In some embodiments, the hexapeptide provides upregulation of HAS2 in keratinocytes and potent downregulation of HYAL2 in keratinocytes. In some embodiments, the compositions described herein stimulate the synthesis of HA in dermal and/or epidermal cells. In some embodiments, the HA synthesized with the application of the composition comprises high MW HA than the HA synthesized without the composition. In some embodiments, the high MW HA produced by stimulation has a MW of at least 0.5 MDa, 1.0 MDa, 1.5 MDa, or 2.0 MDa. In some embodiments, the high MW HA produced by stimulation has a MW of about 1 MDa to 4 MDa. In some embodiments, exposure of dermal cells to octapeptide stimulates the production of HA. In some embodiments, exposure of dermal cells to octapeptide-45 stimulates the production of HA. In some embodiments, exposure of dermal fibroblasts to octapeptide stimulates the production of HA. In some embodiments, exposure of dermal fibroblasts to octapeptide at a concentration of at least about 100 μg/ml stimulates the production of HA. In some embodiments, exposure of epidermal cells to octapeptide stimulates the production of HA. In some embodiments, exposure of epidermal cells to octapeptide-45 stimulates the production of HA. In some embodiments, exposure of epidermal keratinocytes to octapeptide stimulates the production of HA. In some embodiments, exposure of epidermal keratinocytes to octapeptide-45 stimulates the production of HA.

In some embodiments, exposure of dermal fibroblasts to lactoferrin at a concentration of at least about 500 μg/ml stimulates the production of HA. In some embodiments, exposure of dermal fibroblasts to phosphatidylserine at a concentration of at least about 500 μg/ml stimulates the production of HA. In some embodiments, exposure of dermal fibroblasts to Tremella at a concentration of at least about 500 μg/ml stimulates the production of HA. In some embodiments, exposure of dermal fibroblasts to hydroxymethoxphenyl decanone at a concentration of at least about 250 μg/ml stimulates the production of HA.

In some embodiments, exposure of skin to the compositions comprising one or more octapeptides increases expression of CD44. In some embodiments, exposure of skin to the composition comprising octapeptide-45 increases expression of CD44. In some embodiments, exposure of epidermal keratinocytes and/or dermal fibroblasts to the composition increases the expression of CD44 in the exposed cells. In some embodiments, increases in expression of CD44 contribute to the remodeling of dermal ECM. In some embodiments, increases in expression of CD44 contribute to the remodeling of dermal ECM to reverse the appearance of solar elastosis. In some embodiments, increases in expression of CD44 contribute to a reduction in skin redness. In some embodiments, increases in expression of CD44 contribute to a reduction in skin inflammation. In some embodiments, increases in expression of CD44 contribute to a reduction in the appearance of wrinkles. In some embodiments, increases in expression of CD44 contribute to a reduction in the appearance of fine lines. In some embodiments, increases in expression of CD44 contribute to a reduction in the appearance of deep lines. In some embodiments, increases in expression of CD44 contribute to a reduction in the appearance of deep lines in facial skin. In some embodiments, increases in expression of CD44 contribute to a reduction in the appearance of crepiness. In some embodiments, increases in expression of CD44 contribute to a reduction in the appearance of pore size. In some embodiments, increases in expression of CD44 contribute to an improvement in the appearance of skin texture. In some embodiments, increases in expression of CD44 contribute to a reduction in skin dryness. In some embodiments, increases in expression of CD44 contribute to an improvement in skin hydration.

Hyaluronic Acid

In some embodiments described herein, a formulation for use on the skin comprises a hyaluronic acid or a derivative thereof. In some embodiments, the hyaluronic acid comprises high molecular weight hyaluronic acid (high MW HA), or a derivative thereof. In some embodiments, the high MW HA comprises high MW HA having an average molecular weight of at least about 0.5 MDa, 1.0 MDa, 1.5 MDa, 2.0 MDa, 3.0 MDa, 4.0 MDa, or 5.0 MDa. In some embodiments, the high MW HA comprises high MW HA of a molecular weight of about 1 MDa to about 5 MDa. In some embodiments, high MW HA comprises a sodium hyaluronate. In some embodiments, high MW HA comprises a sodium hyaluronate crosspolymer.

### Components of Formulation

In some embodiments described herein, are compositions comprising phosphatidylserine. In some embodiments described herein, are compositions comprising Tremella fuciformis extract. In some embodiments described herein, are compositions comprising hydroxymethoxyphenyl decanone. In some embodiments, a composition comprises a synthetic tripeptide, an octapeptide, a hexapeptide, lactoferrin, phosphatidylserine, Tremella fuciformis extract, or hydroxymethoxyphenyl decanone, or combinations thereof. In some embodiments, the composition provided herein increases production of high MW HA in skin cells. In some embodiments, the composition increases production of high MW HA in epidermal cells. In some embodiments, the composition increases production of high MW HA in dermal cells. In some embodiments, the composition provided herein increases the integrity of the barrier beneath the stratum basale. In some embodiments, the composition increases the thickness of the barrier beneath the stratum basale. In some embodiments, the composition increases expression of genes regulating the barrier function of the stratum basale. In some embodiments, the composition increases the barrier function of skin. In some embodiments, the composition increases the barrier function of skin to prevent excessive water loss. In some embodiments, the composition increases the barrier function of skin to maintain a higher moisture content. In some embodiments, the composition increases the barrier function of skin to increase skin hydration. In some embodiments, the composition increases the barrier function of skin to reduce skin dryness. In some embodiments, the composition increases the barrier function of the skin to reduce redness. In some embodiments, the composition increases the barrier function of the skin to reduce inflammation. In some embodiments, the composition increases the barrier function of the skin to reduce the appearance of wrinkles. In some embodiments, the composition increases the barrier function of the skin to reduce the appearance of fine lines. In some embodiments, the composition increases the barrier function of the skin to reduce the appearance of deep lines in skin. In some embodiments, the composition increases the barrier function of the skin to reduce the appearance of deep lines in facial skin. In some embodiments, the composition increases the barrier function of the skin to reduce the appearance of crepiness. In some embodiments, the composition increases the barrier function of the skin to reduce the appearance of pore size. In some embodiments, the composition increases the barrier function of the skin to improve the appearance of skin texture. In some embodiments, the composition is aqueous. In some embodiments, the composition reduces inflammation as assessed by a reduction or a lack of increase in an expression level of nitric oxide synthase 2 (NOS2), tumor necrosis factor (TNF), interleukin 12 (IL-12b), or cluster of differentiation-80 (CD80), or combinations thereof. In some embodiments, the composition increases CD44 expression. In some embodiments, the composition reduces redness of a skin of an individual after applying the composition. In some embodiments, the composition reduces redness of a skin of an individual after applying the composition and the reduction in redness is measured by photography. In some embodiments, the composition increases skin hydration in a skin of an individual after applying the composition. In some embodiments, the composition increases skin hydration in a skin of an individual after applying the composition. In some embodiments, the composition promotes HA synthesis. In some embodiments, the composition upregulates hyaluronic acid synthase (HAS) expression. In some embodiments, the composition upregulates hyaluronic acid synthase (HAS) expression, wherein the HAS comprises HAS2. In some embodiments, the composition downregulates hyaluronidase expression. In some embodiments, the composition downregulates hyaluronidase expression wherein the hyaluronidase comprises hyaluronidase 2 (HYAL2).

In some embodiments, the composition comprises tripeptide-1, hexapeptide-12, and octapeptide-45. In some embodiments, the composition increases elastin production. In some embodiments, the composition increases production of tropoelastin. In some embodiments, the composition increases production of microfibrillar-associated protein 4 (MFAP4). In some embodiments, the composition upregulates SA1002 expression. In some embodiments, the composition upregulates LAMA3 expression. In some embodiments, the composition upregulates MERTK expression. In some embodiments, the composition upregulates ITGB4 expression. In some embodiments, the composition upregulates gene expression for one or more pathways related to ECM regulation.

In some embodiments, the composition comprises a synthetic tripeptide, an octapeptide, a hexapeptide, lactoferrin, phosphatidylserine, Tremella fuciformis extract, or hydroxymethoxyphenyl decanone, or combinations thereof. In some embodiments, the composition provided herein increases production of high MW HA in skin cells. In some embodiments, the composition increases production of high MW HA in epidermal cells. In some embodiments, the composition increases production of high MW HA in dermal cells. In some embodiments, the compositions provided herein decrease one or more of the appearance of redness, skin inflammation, the appearance of rosacea, the appearance of erythema, the appearance of fine blood vessels in skin, the appearance of wrinkles, the appearance of fine lines, the appearance of deep lines, the appearance of crepiness of skin, the appearance of pore size, excess water loss in the skin, dryness of the skin, or the appearance of solar estastosis. In some embodiments, the compositions provided herein improve the appearance of skin texture and/or increase skin hydration. In some embodiments, the compositions provided herein increase the rate of collagen turnover. In some embodiments, the collagen turnover comprises replacing old thin collagen fibers in dermal ECM into denser, healthier collagen fibers in dermal ECM.

In some embodiments described herein, are topical compositions for reducing inflammation comprising an octapeptide; and a hyaluronic acid (HA) or derivatives thereof, wherein the composition reduces inflammation. In some embodiments, wherein the HA or derivatives thereof has a molecular weight of at least 0.5 MDa. In some embodiments, wherein the HA or derivatives thereof has a molecular weight of at least 1 MDa. In some embodiments, the high MW HA or derivatives comprises sodium hyaluronate. In some embodiments, the sodium hyaluronate comprises a sodium hyaluronate crosspolymer. In some embodiments, the octapeptide comprises at least one of an amino acid sequence GDGDGASA (SEQ ID NO: 1), GPMGPSGP (SEQ ID NO: 2), GLGPGARA (SEQ ID NO: 3), GPQGFQGP (SEQ ID NO: 4), GPHGVREA (SEQ ID NO: 5), GPMGPRGP (SEQ ID NO: 6), GPGKNGDD (SEQ ID NO: 7), or GPMGPRGP (SEQ ID NO: 8). In some embodiments, the octapeptide comprises an amino acid sequence GPHGVREA (SEQ ID NO: 5). In some embodiments, the octapeptide is the amino acid sequence GPHGVREA (SEQ ID NO: 5). In some embodiments, the octapeptide comprises octapeptide-45. In some embodiments, the composition comprises a synthetic tripeptide. In some embodiments, the synthetic tripeptide is tetradecyl aminobutyroylvalylaminobutyric urea trifluoroacetate. In some embodiments, the composition comprises a hexapeptide. In some embodiments, the hexapeptide is hexapeptide-11. In some embodiments, the hexapeptide-11 is encapsulated in a liposome. In some embodiments, the composition comprises lactoferrin. In some embodiments, the lactoferrin is encapsulated in a liposome. In some embodiments, the composition comprises phosphatidylserinc. In some embodiments, the composition comprises Tremella fuciformis extract. In some embodiments, the composition comprises hydroxymethoxyphenyl decanone. In some embodiments, the composition comprises a synthetic tripeptide, an octapeptide, a hexapeptide, lactoferrin, phosphatidylscrinc, Tremella fuciformis extract, or hydroxymethoxyphenyl decanone, or combinations thereof. In some embodiments, the composition is aqueous.

In some embodiments, the reduction in inflammation is assessed by a reduction or a lack of increase in an expression level of nitric oxide synthase 2 (NOS2), tumor necrosis factor (TNF), interleukin 12 (IL-12b), or cluster of differentiation-80 (CD80), or combinations thereof. In some embodiments, the composition increases CD44 expression. In some embodiments, the composition reduces redness of a skin of an individual after applying the composition. In some embodiments, the redness is measured by photography. In some embodiments, the composition increases skin hydration in a skin of an individual after applying the composition. In some embodiments, the composition promotes HA synthesis. In some embodiments, wherein the composition upregulates hyaluronic acid synthase (HAS) expression. In some embodiments, the composition upregulates hyaluronic acid synthase (HAS) expression, wherein the HAS comprises HAS2. In some embodiments, the composition downregulates hyaluronidase expression. In some embodiments, the composition downregulates hyaluronidase expression, wherein the hyaluronidase comprises hyaluronidase 2 (HYAL2).

In some embodiments, the composition reduces the appearance of fine lines. In some embodiments, the composition reduces the appearance of deep lines. In some embodiments, the composition reduces the appearance of deep lines in facial skin. In some embodiments, the composition reduces the appearance of wrinkles. In some embodiments, the composition reduces the appearance of an age spot. In some embodiments, the composition reduces the appearance of crepiness. In some embodiments, the composition reduces the appearance of total area of pore size. In some embodiments, the composition reduces skin dryness. In some embodiments, the composition improves the appearance of skin texture. In some embodiments, the composition improves the appearance of solar elastosis. In some embodiments, the composition reduces or reverses a sign of solar elastosis.

In some embodiments, the compositions provided herein act to increase production of high MW HA in the skin. In some embodiments, the compositions provided herein act to increase the amount of high MW HA in the skin. In some embodiments, the compositions provided herein act to increase production of high MW HA by one or more of epidermal cells, fibroblasts, keratinocytes, or other skin cells. In some embodiments, the compositions provided herein act to increase deposition of high MW HA in the skin ECM. In some embodiments, application of the compositions provided herein result in an increased ratio of high MW HA to low MW HA in the skin. In some embodiments, application of the compositions provided herein result in an increased ratio of high MW HA to low MW HA in the epidermis, dermis, or a combination thereof. In some embodiments, the increased ratio of high MW HA to low MW HA in the skin result in reduced inflammation. In some embodiments, the increased ratio of high MW HA to low MW HA in the skin result in reduced visible redness. In some embodiments, the increased ratio of high MW HA to low MW HA in the skin result in reduced appearance of visible fine lines. In some embodiments, the increased ratio of high MW HA to low MW HA in the skin result in reduced appearance of visible deep lines. In some embodiments, the increased ratio of high MW HA to low MW HA in the skin result in reduced appearance of visible decp lines in the face of an individual. In some embodiments, the increased ratio of high MW HA to low MW HA in the skin result in reduced skin crepiness. In some embodiments, the increased ratio of high MW HA to low MW HA in the skin result in improved skin elasticity. In some embodiments, the increased ratio of high MW HA to low MW HA in the skin result in reduced total area of pore size. In some embodiments, the increased ratio of high MW HA to low MW HA in the skin result in improved visible skin texture. In some embodiments, the increased ratio of high MW HA to low MW HA in the skin result in reduced skin dryness. In some embodiments, the increased ratio of high MW HA to low MW HA in the skin result in increased skin hydration. In some embodiments, the increased ratio of high MW HA to low MW HA in the skin result in reduced visible sign of solar elastosis. In some embodiments, the increased ratio of high MW HA to low MW HA in the skin result in reversal of visible sign of solar clastosis. In some embodiments, the increased ratio of high MW HA to low MW HA in the skin result in remodeling of dermal ECM to produced thicker collagen fibers. In some embodiments, the increased ratio of high MW HA to low MW HA in the skin result in remodeling of dermal ECM to produce denser collagen fibers. In some embodiments, the increased ratio of high MW HA to low MW HA in the skin result in remodeling of dermal ECM into a healthier collagen fiber network. In some embodiments, the increase in high MW HA in the skin results in a decrease in inflammation in the epidermis and an increase in high MW HA production in the dermis.

### Stability Testing

Stability testing of the compositions can be conducted as follows. High temperature testing is now commonly used as a predictor of long-term stability. High temperature testing can be conducted at 37° C. (98° F.) and 45° C. (113° F.). If a product is stored at 45° C. for three months (and exhibits acceptable stability) then it should be stable at room temperature for two years. A good control temperature is 4° C. (39° F.) where most products will exhibit excellent stability. Sometime, the product is subjected to −10° C. (14° F.) for three months.

In some instances, stability of the product is assessed by passing three cycles of temperature testing from −10° C. (14° F.) to 25° C. (77° F.). In such cases, the product is placed at −10° C. for 24 hours and then placed at room temperature (25° C.) for 24 hours. This completes one cycle. An even more rigorous test is a −10° C. to 45° C. five-cycle test. This puts emulsions under a tremendous stress.

In some instances, the dispersed phase (of an oil-in-water emulsion) may separate and rise to the top of the emulsion forming a layer of oil droplets. This phenomenon is called creaming. Creaming is one of the first signs of impending emulsion instability. In some instances, one test method to predict creaming is centrifugation. In some instances, the emulsion is heated to 50° C. (122° F.) and centrifuged for thirty minutes at 3000 rpm. In some instances, the emulsion is inspected for signs of creaming.

In some embodiments, formulas and packaging can be sensitive to the UV radiation. In some instances, the product is placed in glass and the actual package in a light box that has a broad-spectrum output. In some instances, another glass jar completely covered with aluminum foil serves as a control. In some instances, a discoloration of the product may be observed.

For all the above-mentioned tests the color, odor/fragrance, viscosity, pH value, and, if available, particle size uniformity and/or particle agglomeration under the microscope can be observed.

### Methods

### Methods Using Peptide Formulations

Described herein are methods of preparing and using a composition comprising one or more peptides comprising an amino acid sequence feature found in collagen and/or elastin. In some embodiments, the peptide comprises the amino acid sequence features found in elastin. In some embodiments, the peptide comprises the amino acid sequence features found in collagen. In some embodiments, methods comprise topical application of the composition. In some embodiments, the one or more peptides comprise an octapeptide. In some embodiments, the octapeptide comprises an amino acid sequence H-Gly-X₁-X₂-X₃-X₄-X₅-X₆-X₇-OH, wherein X₁ is Pro, Asp, or Leu; wherein X₂ is His, Gly, Met, or Gln; wherein X₃ is Gly, Asp, Pro, or Lys; wherein X₄ is Val, Gly, Pro, Phe, or Asn; wherein X₅ is Arg, Ala, Ser, Gln, or Gly; wherein X₆ is Glu, Ser, Gly, Arg, or Asp; and wherein X₇ is Ala, Pro, or Asp. In some embodiments, the octapeptide comprises an amino acid sequence GPHGVREA, GDGDGASA, GPMGPSGP, GLGPGARA, GPQGFQGP, GPMGPRGP, or GPGKNGDD. In some embodiments, the octapeptide comprises an amino acid sequence GPHGVREA, GDGDGASA, GPMGPSGP, GLGPGARA, GPQGFQGP, GPMGPRGP, or GPGKNGDD, or any combination thereof. In some embodiments, the octapeptide comprises an amino acid sequence GPHGVREA. In some embodiments, the octapeptide comprises a chemical structure of octapeptide-45 (FIG. 22).

Described herein are methods for reducing inflammation in a skin of an individual comprising topical application of one or more peptides with amino acid sequence features found in collagen or elastin. In some embodiments, methods for reducing inflammation in a skin of an individual comprise applying a topical composition comprising an octapeptide. In some embodiments, the octapeptide comprises at least one of an amino acid sequence GDGDGASA (SEQ ID NO: 1), GPMGPSGP (SEQ ID NO: 2), GLGPGARA (SEQ ID NO: 3), GPQGFQGP (SEQ ID NO: 4), GPHGVREA (SEQ ID NO: 5), GPMGPRGP (SEQ ID NO: 6), GPGKNGDD (SEQ ID NO: 7), or GPMGPRGP (SEQ ID NO: 8). In some embodiments, the octapeptide comprises an amino acid sequence GPHGVREA (SEQ ID NO: 5). In some embodiments, the octapeptide comprises octapeptide-45.

In some embodiments, the composition comprises a synthetic tripeptide. In some embodiments, the synthetic tripeptide is tetradecyl aminobutyroylvalylaminobutyric urea trifluoroacetate. In some embodiments, the composition comprises a hexapeptide. In some embodiments, the hexapeptide is hexapeptide-11. In some embodiments, the hexapeptide-11 is encapsulated in a liposome. In some embodiments, the composition comprises lactoferrin. In some embodiments, the lactoferrin is encapsulated in a liposome. In some embodiments, the composition comprises phosphatidylserine. In some embodiments, the composition comprises Tremella fuciformis extract. In some embodiments, the composition comprises hydroxymethoxyphenyl decanone. In some embodiments, the composition comprises a synthetic tripeptide, an octapeptide, a hexapeptide, lactoferrin, phosphatidylserine, Tremella fuciformis extract, or hydroxymethoxyphenyl decanone, or combinations thereof. In some embodiments, the composition is aqueous. In some embodiments, the reduction in inflammation is assessed by a reduction or a lack of increase in an expression level of nitric oxide synthase 2 (NOS2), tumor necrosis factor (TNF), interleukin 12 (IL-12b), or cluster of differentiation-80 (CD80), or combinations thereof. In some embodiments, the composition increases CD44 expression. In some embodiments, the composition reduces redness of a skin of an individual after applying the composition. In some embodiments, the redness is measured by photography.

### Methods to Increase Elastin Production

In some embodiments, the present disclosure relates to methods or treating or restoring aging skin or increasing elastin production in the skin, such methods comprising administering a topical composition disclosed herein to the skin. In some embodiments, the composition comprises tripeptide-1, hexapeptide-12, and octapeptide-45. In some embodiments, the composition increases elastin production. In some embodiments, the composition increases production of tropoelastin. In some embodiments, the composition increases production of microfibrillar-associated protein 4 (MFAP4). In some embodiments, the composition upregulates SA1002 expression. In some embodiments, the composition upregulates LAMA3 expression. In some embodiments, the composition upregulates MERTK expression. In some embodiments, the composition upregulates ITGB4 expression. In some embodiments, the composition upregulates gene expression for one or more pathways related to ECM regulation.

### Methods to Increase Skin Hydration

In some embodiments, a composition increases skin hydration in a skin of an individual after applying the composition. In some embodiments, the skin hydration in the skin of an individual is measured at baseline. In some embodiments, the skin hydration in the skin of an individual is measured at Week 1, 2, 3, 4, 5, 6, 7, 8, 9, or 10 after initiation of a skin treatment routine comprising application of a topical composition. In some embodiments, an individual completes a self-assessment of skin hydration at baseline. In some embodiments, an individual completes a self-assessment of skin hydration at Week 1, 2, 3, 4, 5, 6, 7, 8, 9, or 10 after initiation of a skin treatment routine comprising application of a topical composition. In some embodiments, an investigator completes an assessment of skin hydration of an individual at baseline. In some embodiments, an investigator completes an assessment of skin hydration of an individual at Week 1, 2, 3, 4, 5, 6, 7, 8, 9, or 10 after initiation of a skin treatment routine comprising application of a topical composition. In some embodiments, an individual has a measurement taken on a side of the face using a skin hydration sensor measurement system by Wearifi. In some embodiments, an individual has a measurement taken at baseline. In some embodiments, an individual has a measurement taken at Week 1, 2, 3, 4, 5, 6, 7, 8, 9, or 10 after initiation of a skin treatment routine comprising application of a topical composition. In some embodiments, an individual has a skin hydration measurement taken 15 minutes post cleansing the facial skin of the individual. In some embodiments, an individual has a skin hydration measurement taken following a first 15 minutes post cleansing the facial skin of the individual and a second 15 minutes post application of a composition. In some embodiments, methods are described wherein an individual has a skin hydration measurement taken three times on a side of the face. In some embodiments, an individual has a skin hydration measurement taken three times on a side of the face in the same facial area. In some embodiments, an individual has a skin hydration measurement taken on the right and left sides of the face. In some embodiments, an individual has a skin hydration measurement taken on the right and left sides of the face, three times on each side. In some embodiments, an individual has a skin hydration measurement taken on the right and left sides of the face, three times on each side in the same facial area. In some embodiments, an individual has a skin hydration measurement that captures the water content across stratum corneum and upper epidermis. In some embodiments, an individual has a skin hydration measurement wherein the measurement is a quantitative result of volumetric ratio of water in skin tissue. In some embodiments, an individual has a skin hydration measurement wherein the measurement represents an absolute value with direct relevance to the effective hydration of the skin. In some embodiments, an individual has a skin hydration measurement taken at baseline and after initiation of a skin treatment routine comprising application of a topical composition.

### Methods of Assessing the Effects of Topical Application

In some embodiments, an individual completes a self-assessment survey on features of their facial skin. In some embodiments, the survey is completed at baseline, at Week 1, 2, 3, 4, 5, 6, 7, 8, 9, or 10. In some embodiments, the survey is completed post-cleansing the skin. In some embodiments, the survey is completed about 15 minutes post-cleansing the skin. In some embodiments, the individual completes the survey after initiation of a skin treatment routine comprising application of a topical composition. In some embodiments, the individual completes the survey using a scale to rate the assessment of their skin. In some embodiments, the individual completes the survey using a 5-point scale to rate the assessment of their skin. In some embodiments, the individual completes a self-assessment survey on features of their facial skin at baseline and after initiation of a skin treatment routine comprising application of the composition.

In some embodiments, an individual completes a self-assessment survey on overall satisfaction with the composition. In some embodiments, an individual completes a self-assessment survey on overall satisfaction with the composition. In some embodiments, an individual completes a self-assessment survey on overall satisfaction with the composition at baseline and at other time points. In some embodiments, an individual completes a self-assessment survey on overall satisfaction with the composition at baseline and Week 1, 2, 3, 4, 5, 6, 7, 8, 9, or 10. In some embodiments, an overall satisfaction survey uses a scale compared to baseline. In some embodiments, an overall satisfaction survey uses a 7-point scale compared to baseline.

In some embodiments, an investigator completes an assessment on the facial skin of an individual. In some embodiments, the individual initiates a skin treatment routine comprising application of the composition provided herein. In some embodiments, methods are described wherein an investigator completes an assessment on the facial skin of an individual at baseline and a later time point. In some embodiments, the later time point is Week 1, 2, 3, 4, 5, 6, 7, 8, 9, or 10 after the first application. In some embodiments, an investigator completes an assessment on the facial skin of an individual post cleansing of the skin. In some embodiments, an investigator completes an assessment on the facial skin of an individual about 15 minutes post cleansing of the skin. In some embodiments, an investigator completes an assessment on the facial skin of an individual to assess fine lines/wrinkling, crepiness, texture, erythema, dryness or moisture/hydration. In some embodiments, an investigator completes an assessment on the facial skin of an individual to assess fine lines/wrinkling, crepiness, texture, erythema, dryness and moisture/hydration. In some embodiments, a scale is used to assess fine lines/wrinkling, crepiness, texture, erythema, dryness and/or moisture/hydration. In some embodiments, a 10-point scale is used to assess fine lines/wrinkling, crepiness, texture, erythema, dryness and/or moisture/hydration. In some embodiments, the scale parameters comprise: 0 absent; 1-3 mild, 4-6 moderate, 7-9 severe. In some embodiments, methods are described wherein the results of the investigator assessment are subjected to statistical analysis.

In some embodiments, as described herein, are methods wherein a photograph is taken of an individual who has initiated a skin treatment routine comprising application of the composition provided herein. In some embodiments, photographs are taken showing the facial skin of an individual at baseline and at Week 2, Week 4, and/or Week 8 following initiation of a skin treatment routine comprising application of the composition. In some embodiments, a portrait lens (85 mm f/1.4) is used as part of an imaging system to capture the facial skin features of an individual. In some embodiments, a photograph is taken post-cleansing of the skin. In some embodiments, a photograph is taken about 15 minutes post-cleansing of the skin. In some embodiments, a photograph is taken post-cleansing of the skin and post-application of a topical study composition. In some embodiments, a photograph is taken following a first time period of about 15 minutes post-cleansing of the skin and followed by a second time period of about 15 minutes post-application of a topical study composition. In some embodiments, a VISIA® Skin Analysis System is used to take photographs. In some embodiments, a VISIA® Skin Analysis System is used to conduct analysis of skin features on photographs taken. In some embodiments, a Life Viz® Infinity System is used to take photographs. In some embodiments, a LifeViz® Infinity System is used to conduct analysis of skin features on photographs taken. In some embodiments, a Life Viz® Micro System is used to take photographs. In some embodiments, Life Viz® Micro System is used to conduct analysis of skin features on photographs taken. In some embodiments, skin redness visible in photographs of an individual is assessed. In some embodiments, skin inflammation visible in photographs of an individual is assessed. In some embodiments, skin wrinkles visible in photographs of an individual are assessed. In some embodiments, fine lines visible in photographs of an individual are assessed. In some embodiments, deep lines visible in photographs of an individual are assessed. In some embodiments, deep lines in facial skin visible in photographs of an individual are assessed. In some embodiments, crepiness in facial skin visible in photographs of an individual is assessed. In some embodiments, texture of facial skin visible in photographs of an individual is assessed. In some embodiments, pore size area of facial skin visible in photographs of an individual is assessed. In some embodiments, rosacea of facial skin visible in photographs of an individual is assessed. In some embodiments, fine blood vessel of facial skin visible in photographs of an individual are assessed. In some embodiments, solar clastosis of facial skin visible in photographs of an individual is assessed. In some embodiments, an age spot on facial skin visible in photographs of an individual is assessed.

In some embodiments, a punch biopsy may be taken periauricularly at baseline, and post application at week 2, week 4 and week 8. In some embodiments, an individual may be instructed to apply a topical serum periauricular twice a day for the duration of the study. In some embodiments, an independent dermatopathologist evaluates the tissue pre and post application of a study product. In some embodiments, methods are described wherein biopsy samples are processed for histological analysis. In some embodiments, the histological analysis comprises CD44 immunohistochemistry, hemotoxylin and cosin staining. In some embodiments, hemotoxylin and cosin staining reveal the structure of dermal extracellular matrix and a thickness and density of collagen fibers. In some embodiments, CD44 immunohistochemistry reveal a cellular location of HA production.

In some embodiments, the topical application of composition reduces inflammation in an individual. In some embodiments, methods are described wherein the topical application of the composition reduces skin inflammation in an individual when comparing baseline or another time point to Week 1, 2, 3, 4, 5, 6, 7, 8, 9, or 10. In some embodiments, the topical application of composition reduces skin redness in an individual. In some embodiments, methods are described wherein the topical application of the composition reduces skin redness in an individual when comparing baseline or another time point to Week 1, 2, 3, 4, 5, 6, 7, 8, 9, or 10. In some embodiments, the topical application of composition reduces the appearance of deep lines in an individual. In some embodiments, methods are described wherein the topical application of the composition reduces the appearance of deep lines in an individual when comparing baseline or another time point to Week 1, 2, 3, 4, 5, 6, 7, 8, 9, or 10. In some embodiments, the topical application of composition reduces the appearance of wrinkles in an individual. In some embodiments, methods are described wherein the topical application of the composition reduces the appearance of wrinkles in an individual when comparing baseline or another time point to Week 1, 2, 3, 4, 5, 6, 7, 8, 9, or 10. In some embodiments, the topical application of composition reduces the appearance of crepiness in an individual. In some embodiments, methods are described wherein the topical application of the composition reduces the appearance of crepiness in an individual when comparing baseline or another time point to Week 1, 2, 3, 4, 5, 6, 7, 8, 9, or 10. In some embodiments, the topical application of composition reduces skin dryness in an individual. In some embodiments, methods are described wherein the topical application of the composition reduces the skin dryness in an individual when comparing baseline or another time point to Week 1, 2, 3, 4, 5, 6, 7, 8, 9, or 10.

In some embodiments, the topical application of composition increases skin hydration in an individual. In some embodiments, methods are described wherein the topical application of the composition increases skin hydration in an individual when comparing baseline or another time point to Week 1, 2, 3, 4, 5, 6, 7, 8, 9, or 10. In some embodiments, the topical application of composition improves the appearance of skin texture in an individual. In some embodiments, methods are described wherein the topical application of the composition improves the appearance of skin texture in an individual when comparing baseline or another time point to Week 1, 2, 3, 4, 5, 6, 7, 8, 9, or 10. In some embodiments, the topical application of composition improves the appearance of solar elastosis in an individual. In some embodiments, methods are described wherein the topical application of the composition improves the appearance of solar elastosis in an individual when comparing baseline or another time point to Week 1, 2, 3, 4, 5, 6, 7, 8, 9, or 10. In some embodiments, the topical application of composition reverses the appearance of solar elastosis in an individual. In some embodiments, methods are described wherein the topical application of the composition reverses the appearance of solar elastosis in an individual when comparing baseline or another time point to Week 1, 2, 3, 4, 5, 6, 7, 8, 9, or 10.

In some embodiments, the topical application of composition stimulates HA synthesis in the skin of an individual. In some embodiments, methods are described wherein the topical application of the composition stimulates HA synthesis in the skin of an individual when comparing baseline or another time point to Week 1, 2, 3, 4, 5, 6, 7, 8, 9, or 10. In some embodiments, the topical application of composition upregulates hyaluronic acid synthase (HAS) expression in the skin of an individual. In some embodiments, methods are described wherein the topical application of the composition upregulates hyaluronic acid synthase (HAS) expression in the skin of an individual when comparing baseline or another time point to Week 1, 2, 3, 4, 5, 6, 7, 8, 9, or 10. In some embodiments, the stimulation of hyaluronic acid synthase (HAS) activity in a skin of an individual comprises an increase in expression of HAS2. In some embodiments, the topical application of the composition downregulates hyaluronidase expression in the skin of an individual. In some embodiments, methods are described wherein the topical application of the composition downregulates hyaluronidase expression in the skin of an individual when comparing baseline or another time point to Week 1, 2, 3, 4, 5, 6, 7, 8, 9, or 10. In some embodiments, the composition downregulates hyaluronidase expression in a skin of an individual comprises a decrease in expression of HYAL2.

In some embodiments, the topical application of the composition reduces appearance of a bruise, an aging spot, or a wrinkle in a skin of an individual. In some embodiments, methods are described wherein the topical application of the composition reduces appearance of a bruise, an aging spot, or a wrinkle in a skin of an individual when comparing baseline or another time point to Week 1, 2, 3, 4, 5, 6, 7, 8, 9, or 10. In some embodiments, the reduction in appearance of a bruise, an aging spot, or a wrinkle persists after the period of topical application of the composition has ended.

In some embodiments, the topical application of composition increases elastin production in the skin of an individual. In some embodiments, methods are described wherein the topical application of the composition stimulates elastin production in the skin of an individual when comparing baseline or another time point to Week 1, 2, 3, 4, 5, 6, 7, 8, 9, or 10. In some embodiments, the topical application of the composition upregulates MFAP4 expression. In some embodiments, the topical application of the composition increases tropoelastin production. In some embodiments, methods are described wherein the topical application of the composition upregulates MFAP4 expression and/or tropoelastin production in the skin of an individual when compared to baseline or another time point to Week 1, 2, 3, 4, 5, 6, 7, 8, 9, or 10. In some embodiments, the composition upregulates expression of S100A2, LAMA3, MERTK, or ITGB4. In some embodiments, methods are described wherein the topical application of the composition upregulates expression of S100A2, LAMA3, MERTK, or ITGB4 in the skin of an individual when compared to baseline or another time point to Week 1, 2, 3, 4, 5, 6, 7, 8, 9, or 10. In some embodiments, the increase in elastin production, increase in tropoclastin production or upregulation of MFAP4, S100A2, LAMA3, MERTK, or ITGB4 persists after the period of topical application of the composition has ended.

In some embodiments, as described herein, are methods wherein topical application of composition is applied 1, 2, 3, 4, 5, 6, 7, or 8 times a day. In some embodiments, the topical application is applied in the AM. In some embodiments, the topical application is applied in the PM. In some embodiments, the topical application is applied in the AM and PM. In some embodiments, the topical application is applied in the AM or PM. In some embodiments, the topical application is applied post-cleansing of the skin. In some embodiments, the topical application is applied a certain time period after post-cleansing of the skin. In some embodiments, the topical application is applied about 15 minutes post-cleansing of the skin.

In another aspect, which may be combined with any other aspect or embodiment, the present disclosure relates to a method for promoting wound healing, comprising administering the topical composition according to the present disclosure to the skin of a subject.

In another aspect, which may be combined with any other aspect or embodiment, the present disclosure relates to a method for promoting wound healing or wound closure, comprising administering the topical composition according to the present disclosure to the skin of a subject.

In another aspect, which may be combined with any other aspect or embodiment, the present disclosure relates to a method for treating a cutaneous wound, comprising administering the topical composition according to the present disclosure to the skin of a subject.

In some embodiments, the wound (e.g., cutaneous wound) is related to use of a device used for anti-aging indications. In some embodiments, the anti-aging indications may include combatting skin laxity, combatting diminished skin texture and tone, reducing redness, reducing skin blemishes, reducing inflammation, reducing swelling, reducing itching of skin, or any combination thereof.

In another aspect, which may be combined with any other aspect or embodiment, the present disclosure relates to a method for promoting elastogenesis, comprising administering the topical composition according to the present disclosure to the skin of a subject.

In another aspect, which may be combined with any other aspect or embodiment, the present disclosure relates to a method for reducing scarring, comprising administering the topical composition according to the present disclosure to the skin of a subject.

In another aspect, which may be combined with any other aspect or embodiment, the present disclosure relates to a method for reducing the effects of aging on skin, comprising administering the topical composition according to the present disclosure to the skin of a subject.

In another aspect, which may be combined with any other aspect or embodiment, the present disclosure relates to a method for reducing or reversing aging of skin, comprising administering the topical composition according to the present disclosure to the skin of a subject.

In another aspect, which may be combined with any other aspect or embodiment, the present disclosure relates to a method for reducing skin laxity, comprising administering the topical composition according to the present disclosure to the skin of a subject.

In another aspect, which may be combined with any other aspect or embodiment, the present disclosure relates to a method for improving skin texture or improving skin tone, comprising administering the topical composition according to the present disclosure to the skin of a subject.

In another aspect, which may be combined with any other aspect or embodiment, the present disclosure relates to a method for improving pigmentation, comprising administering the topical composition according to the present disclosure to the skin of a subject.

In another aspect, which may be combined with any other aspect or embodiment, the present disclosure relates to a method for reducing redness of skin, comprising administering the topical composition according to the present disclosure to the skin of a subject.

In another aspect, which may be combined with any other aspect or embodiment, the present disclosure relates to a method for reducing skin blemishes or improving the appearance of skin blemishes, comprising administering the topical composition according to the present disclosure to the skin of a subject.

In another aspect, which may be combined with any other aspect or embodiment, the present disclosure relates to a method for reducing inflammation of skin, comprising administering the topical composition according to the present disclosure to the skin of a subject.

In another aspect, which may be combined with any other aspect or embodiment, the present disclosure relates to a method for reducing swelling of skin, comprising administering the topical composition according to the present disclosure to the skin of a subject.

In another aspect, which may be combined with any other aspect or embodiment, the present disclosure relates to a method for reducing itching of skin, comprising administering the topical composition according to the present disclosure to the skin of a subject.

In another aspect, which may be combined with any other aspect or embodiment, the present disclosure relates to a method for promoting extracellular matrix (ECM) remodeling, comprising administering the topical composition according to the present disclosure to the skin of a subject.

In any of the aspects or embodiments disclosed herein, the skin affected by itching is not suffering from one or more of: a wound, skin damage, signs of aging, bruising (e.g., suffering from a bruise related to a body procedure such as injection of a filler), inflammation, redness, soreness, sensitivity, dryness, laxity, crepiness, poor body contouring, discoloration, swelling, a pigmentation disorder (e.g., hyperpigmentation), age spots, wrinkles, pain, discomfort, or dermatoporosis.

In some embodiments, the individual receiving the topical application is a human.

### Kits

In some embodiments, provided herein are kits comprising the composition comprising the peptides described herein. In some embodiments, kits can be provided to an administering physician, other health care professional, a patient, or a caregiver. In some embodiments, a kit comprises a container which contains the compositions in a suitable topical composition, and instructions for administering the composition to a subject. The kit can contain one or more additional therapeutic or other agents. For example, a kit containing a composition in topical form can be provided along with other skin care agents, such as, cleansers, occlusive moisturizers, penetrating moisturizers, sunscreens, sunblocks, and the like. The kit may comprise the composition in bulk form or may comprise separate doses of the composition for serial or sequential administration. The kit can optionally contain one or more diagnostic tools, administration tools, and/or instructions for use. The kit can contain suitable delivery devices, such as, syringes, pump dispensers, single dose packets, and the like, along with instructions for administering the peptide compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

In some embodiments, the composition stimulates the skin regenerating process and assists in improving the skin's appearance, and skin tightness. In some embodiments, the composition is suitable for all skin types and post-procedure skin. In some embodiments, the composition may be provided to the patient in bulk form, to permit a suitable amount of the peptides to be self-administered by the patient. In some embodiments, the patient can apply an amount of the composition sufficient to provide an even coating over the affected area or as otherwise instructed by the physician. In some embodiments, a cleanser, a sunblock, a sunscreen, a penetrating moisturizer, and/or an occlusive moisturizer can be provided for administration before or after the topical composition of the embodiments.

In some embodiments, the various examples of creams, ointments, lotions, solutions, gels, sprays and patches may incorporate the compositions as described herein, in combination with penetration enhancing agents and other agents to improve skin health.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

### EXAMPLES

The following illustrative examples are representative of embodiments of the stimulation, systems, and methods described herein and are not meant to be limiting in any way.

### Example 1. Gene Expression Studies

Provided herein is an example showing the effects of the different active agents in gene expression in fibroblasts and keratinocytes.

### Methods

Cell Lines. Human adult dermal fibroblast and keratinocyte cell lines were treated with 11 different compound treatments (plus DMSO control as treatment #12Primary cells were plated in cell specific media at 5K or 10K cells per cm². Cells were plated in 48 well plates (Greiner) in triplicate, one cell line per plate. Media volume was 500 ul per well. Outer two columns of wells were not used. All rows were used. Cells were cultured for 2 days in 37° C. 5% CO₂ incubator. After 48 hours all the cell cultures were uniform and appeared healthy, no appreciable number of floating dead cells or evidence of vacuolization of cells that might indicate apoptotic or dying cells.

Compounds. Stocks of compounds 1 through 8 on the list below were prepared in PBS. A 50 mg/ml stock of compound 9 (“Phos”; phosphatidylserine) was prepared in DMSO but did not fully go into solution with extensive vortexing and warming. This stock was 100× of final concentration. A 20 mg/ml stock of compound 10 (CBD) was prepared in DMSO. No undissolved particulates were observed but the stock solution was slightly cloudy, not completely clear. This stock was 200× of final concentration.

### Cell Treatments:

1. Lactoferrin (Lacto)
2. TCVRRAF (LCV)
3. Tripeptide-1 (Tri)
4. Hexapeptide-12 (Hex12)
5. Tripeptide-1 and Hexapeptide-12 (TriHex)
6. Hexapeptide-11 (Hex11)
7. Tranexamic acid 5% (Tran Acid)
8. Octapeptide (Octa)
9. Phosphatidylserine (Phos)
10. CBD

Dosing. After 48 hours of attachment culture, fibroblasts and keratinocytes were treated with the test compounds. Compounds were resuspended in the appropriate cell media at a final concentration, and the attachment culture media removed and compound containing media added. The cells were exposed to the following compounds for 24 hr: Lactoferrin (500 μg/ml), TCVRRAF (100 μg/ml) (amino acid sequence extracted from Lactoferrin), Tri-peptide-1-100 ppm (2.9 g/ml), Hexapeptide 12, 100 ppm (2.9 μg/ml), TriHex combination 200 ppm (2.9 μg/ml each), Hexapeptide 11 (100 μg/ml), Tranexamic acid 5% (500 μg/ml), Octapeptide (100 μg/ml)—proprietary peptide designed by R&D at Alastin (Alastin Skincare, Inc., a Galderma Company, Carlsbad CA), Phosphatidylserine 500 μg/ml, or Cannabidiol (CBD) 100 μg/ml. The control cells were left untreated.

RNA Lysate preparation. After 24 hours of compound exposure, the media was removed, the cells were washed 1× with PBS. 100 ul of RNA Lysis Buffer (Takara Bio Cat Num 635013, “10×RNA lysis buffer”, diluted to 1×) was added to the well and mixed thoroughly by trituration, combined in RNAse free microcentrifuge tubes and immediately frozen at −30 C. Samples were prepared from one cell line (one plate) at a time. Plate array is 12×4, with each treatment in a row of 3 wells. The triplicate wells were lysed and combined into a tube in a PCR tube strip. All samples were shipped frozen on dry ice for RNA extraction, library construction and sequencing to 25M paired end 100 bp reads per sample.

RNA-Seq. All the RNA samples were shipped frozen on dry ice for RNA extraction, library construction and sequencing to 25M paired end 100 bp reads per sample. Differentially expressed genes were identified and pathway enrichment was assessed using the Reactome Pathway.

### Results

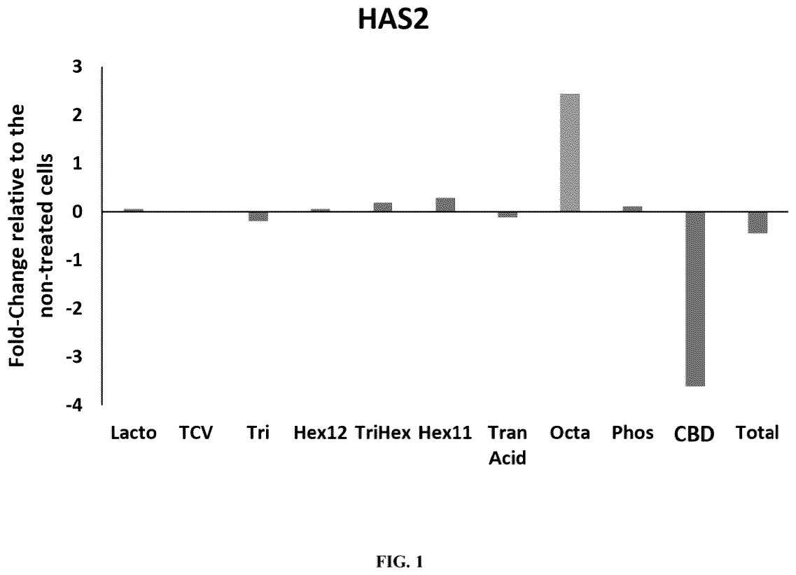

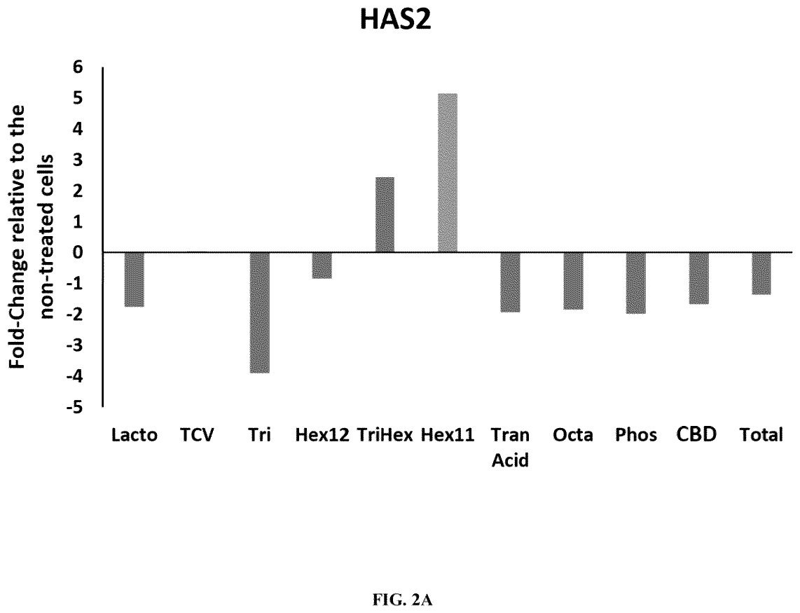

FIG. 1 shows data of Hyaluronic Acid Synthase 2 (HAS2), which is a primary stimulant of HA in fibroblasts, in fibroblasts treated with the various compounds. The data are presented as the fold-change in gene expression relative to the non-treated cells. Octapeptide showed excellent stimulation of Hyaluronic Acid Synthase 2 (HAS2) (FIG. 1).

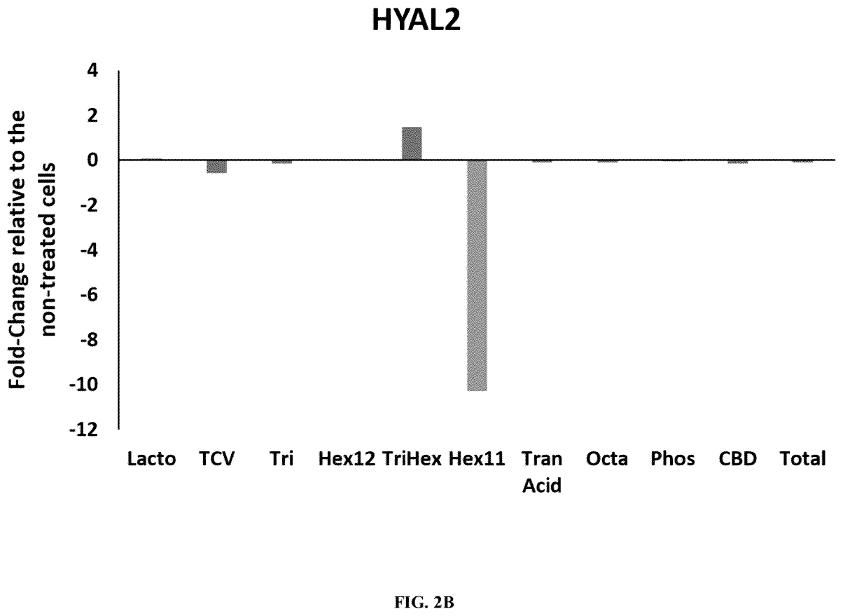

FIG. 2A shows data of HAS2 in keratinocytes treated with the various compounds. The data are presented as the fold-change in gene expression relative to the non-treated cells.

FIG. 2B shows data of Hyaluronidase 2 (HYAL2), which is a HA reducing enzyme, in keratinocytes treated with the various compounds. Hexapeptide-11 showed excellent upregulation of HAS2 in keratinocytes and potent downregulation of HYAL2 in keratinocytes (FIG. 2A-B). Octapeptide showed modest downregulation of HYAL2 in keratinocytes. The data are presented as the fold-change in gene expression relative to the non-treated cells.

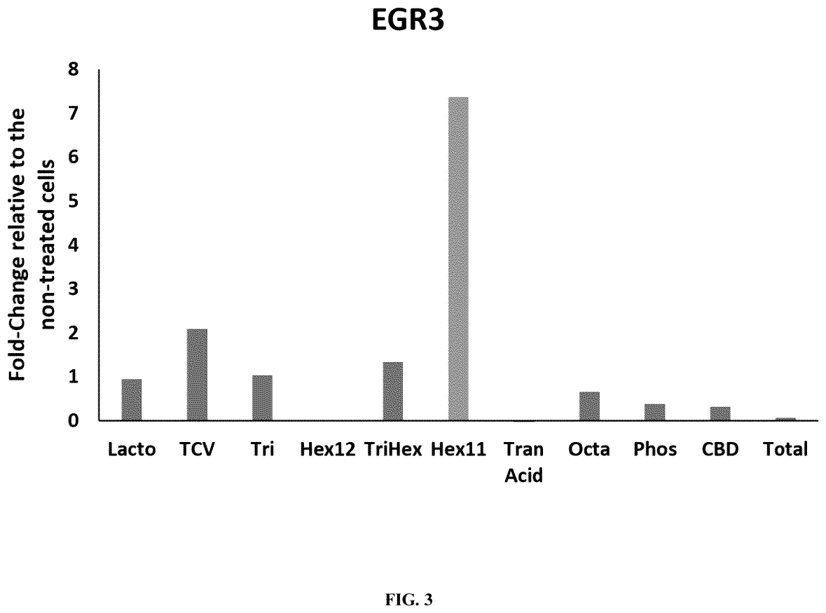

FIG. 3 shows data of Early Growth Response 3 (EGR3), in keratinocytes treated with the various compounds. HEX11 induced a marked upregulation of EGR3 of about seven-fold. EGR3 is a late epidermal regulator of differentiation highly expressed in the stratum granulosum that regulates expression of skin barrier genes and can function to strengthen the barrier of the skin. The data are presented as the fold-change in gene expression relative to the non-treated cells.

This Example shows that the peptides described herein are involved in regulating gene expression of genes involved in hyaluronic acid stimulation, hyaluronic acid turnover and maintenance, integrity and strength of the epidermal skin barrier.

### Example 2. Selection of Agents for HA Production and Size Assessment In Vitro

Provided herein is an example whether octapeptide, SymDecanox™, Tremella, lactoferrin, phosphatidylserine, Hylasome™, Aquaxyl™ and full formulation stimulate the secretion of high-molecular weight (high MW) hyaluronic acid (HA) from dermal fibroblasts (and keratinocytes). Provided herein are assays for HA synthesis.

Using agents identified by RNA-seq as novel HA production stimulants in the fibroblasts, together with other active agents previously determined to stimulate HA production, the production of HA by fibroblasts was confirmed and the molecular weight (MW)/size of the produced HA was determined. The components selected and treatment concentrations used are listed in Table 1. The rationale for the selection of these agents is described here.

### Selection of Agents for HA Production and Size Assessment In Vitro.

Usually, octapeptide (proprietary peptide) upregulates Hyaluronic Acid Synthase-2 (HAS2) gene expression in fibroblasts (based on RNA-Seq data described in Example 1).

Lactoferrin may provide wound healing attributes, promotes fibroblast proliferation and increases HA secretion.

Syn-Hycan is a synthetic tripeptide that stimulates HA. Published evidence suggests that a synthetic tripeptide tetradecyl aminobutyroylvalylaminobutyric urea trifluoroacetate restores facial skin volume by stimulating HA synthesis. Syn-Hycan increases HA and CD44 in the skin in vitro and ex vivo.

### Phosphatidylserine Promotes HA Synthesis.

Sodium Hyaluronate Crosspolymer (Hyalasome) is an extremely high MW synthetic HA with exceptionally high water-binding capacity resulting in excellent moisturizing abilities.

Tremella fuciformis extract derived from an edible mushroom and may provide high levels of moisture from as a natural HA stimulant and has antioxidant properties.

Hydroxymethoxyphenyl decanone is an HA booster, antioxidant, and anti-irritant. It may stimulate the dermal and epidermal hyaluronic acid levels by 259% and 198% versus placebo, respectively, in an ex vivo human skin model.

Hexapeptide-11 may upregulate HAS2 and downregulate the HA reducing enzyme hyaluronidase 2 (HYAL2) in keratinocytes (based on RNA-seq data in Example 1).

TABLE 1
Compounds Used to Treat Fibroblasts
for HA Production Assessment by PAGE

| Sample | | Concentration Used For |
|---|---|---|
| Number | Compound | Respective Sample Numbers |
| 1 | Octapeptide | 100 μg/ml |
| 2 | Lactoferrin | 500 μg/ml |
| 3 | SynHycan | 500 μg/ml |
| 4 | Phosphatidylserine | 500 μg/ml |
| 5 | Hyalasome | 500 μg/ml |
| 6 | Tremella | 500 μg/ml |
| 7 | Hydroxymethoxphenyl decanone | 250 μg/ml |
| 8 | Aquaxyl | 500 μg/ml |
| 9 | Mix of all compounds | 12.5% of the above |
| | | concentrations |
| 10 | Mix of all compounds | 40% of the above |
| | | concentrations |
| 11 | No Treatment | |

### HA Production and Size Assessment In Vitro

Dermal fibroblasts were cultured in growth medium until near confluence in 6-well plates. When they reached confluence, the growth medium was replaced with serum-free medium for 24 hours. Then, the cells were treated with the compounds indicated in Table 1. After 72 hr, 100 μl of media was collected from each treatment condition. The media was concentrated in a SpeedVac concentrator to a final volume of ˜10 μl. All 10 μl from each condition, were loaded onto an acrylamide gel (NuPAGE 4-12% Bis-Tris Protein Gel, Invitrogen, Waltham, MA). Sodium Hyaluronate, Research Grade, (HA2M) from Lifecore Biomedical (Chaska, MN) was reconstituted as recommended by the manufacturer and served as a MW reference, representing high MW HA. The gel was run at 200V for 3 hours to separate MW sizes from proteins and ECM components isolated from the supernatants along with the HA2M reference as a comparator. Gel buffer was exchanged with fresh room temperature buffer every 20 minutes to avoid overheating. The gel was stained using Stains-All (Millipore Sigma, Burlington, MA) and destained according to the manufacturer's protocol. A band running at the same size of the HA2M band indicated the production of high MW HA.

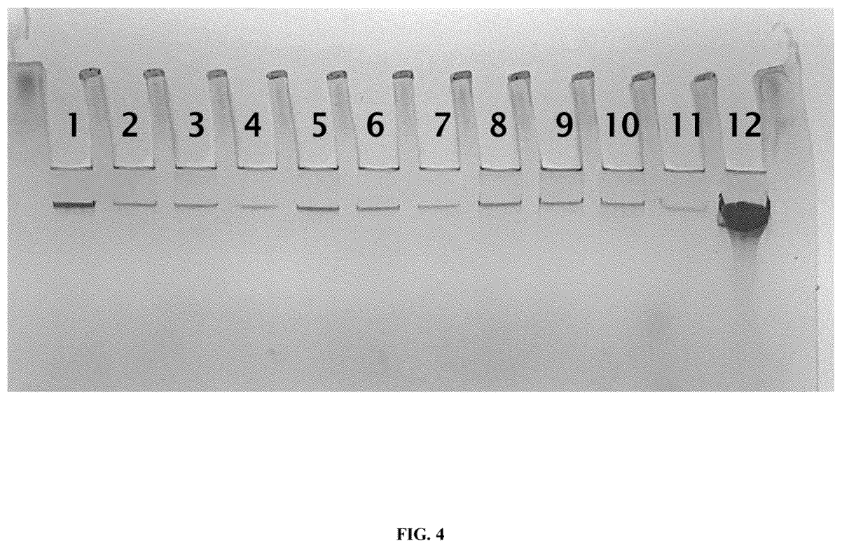

Data is seen in FIG. 4 showing the effect of several compounds on hyaluronic acid production in human dermal fibroblast cells 72 hours after treatment. Number 1-10 are the name of the compounds listed above in Table 1. Number 11 is the concentrated supernatant of untreated cells. Number 12 is the HA2M control (2 MDa/high MW HA) shown as a reference point. FIG. 4 demonstrates fibroblasts producing HA in a range at 2 MDa/high MW HA and none produce low molecular weight (LMW) HA.

### Confirmation of HA

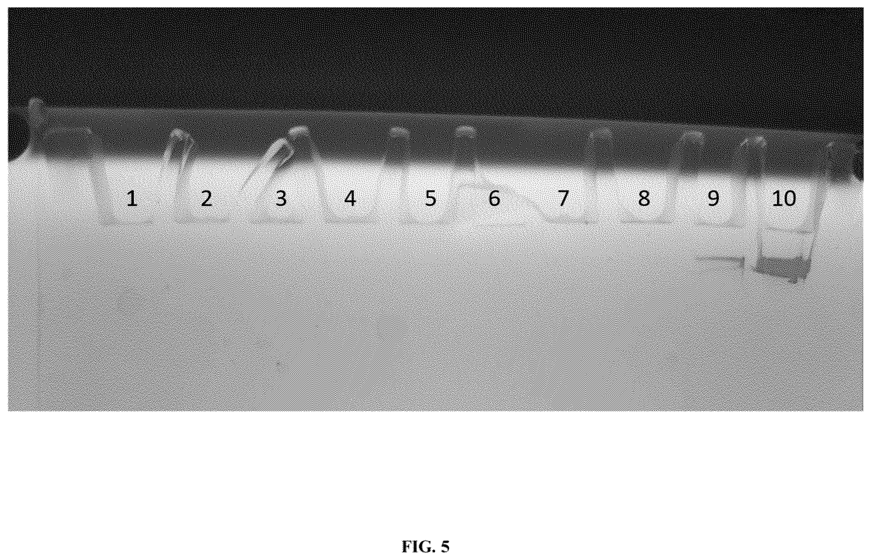

Fibroblasts were cultured as described above until near confluence in 6-well plates. Then, the cells were treated with the compounds indicated in Table 1. After 72 hr, 100 μl of media was collected from each treatment condition. The media was then subjected to hyaluronidase enzyme treatment (1 mg/ml) at 37° C. for 2 hr. One condition was left non-digested (octapeptide at 100 μg/ml). The samples were run on an SDS-PAGE gel, stained, and destained as described above. Results are shown in FIG. 5. Lanes 1-8 correspond to treatment using sample numbers 1-8 listed in Table 1. Lane 9 corresponds to octapeptide treatment at 100 μg/ml left undigested. Lane 10 is the HA2M control (2 MDa high MW HA) shown as a reference point. The absence of the band after digestion indicated that the band identified in the prior step was HA having a high molecular weight (of about 2 MDa).

### Octapeptide Stimulates High MW HA in a Dose-Response Manner in Dermal Fibroblasts.

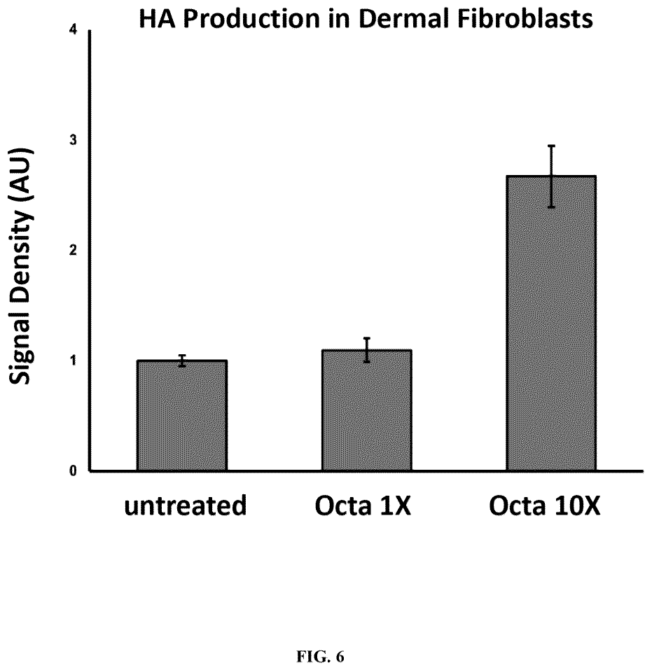

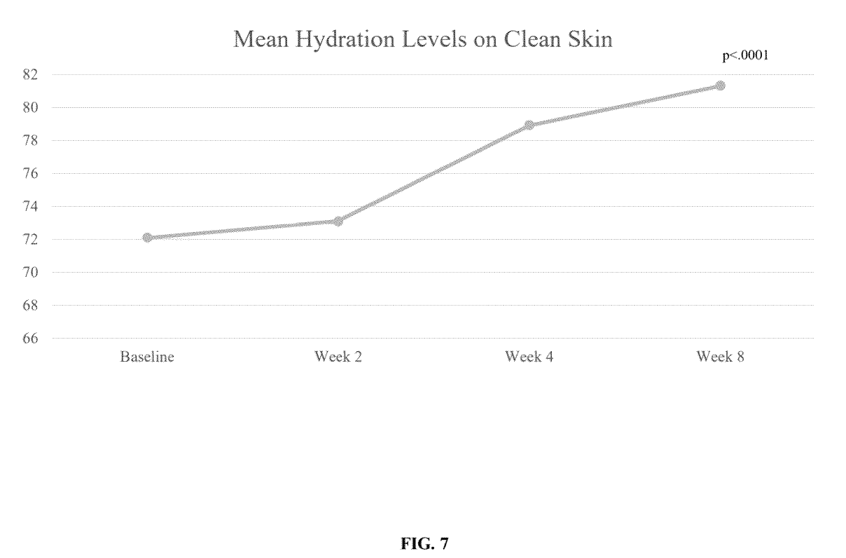

Human dermal fibroblasts were treated with Octapeptide (1× and 10×; corresponding to 100 μg/ml and 1000 μg/ml respectively) or left untreated. After 72 hr, the supernatant was collected, concentrated, run on an SDS-PAGE, stained and imaged. The experiment was conducted 4 times and the density of the bands from each supernatant were quantified (arbitrary units (AU)) using ImageJ. The data was graphed in FIG. 6 and represents the mean±standard deviation of the band densities for each treatment condition. The vast increase in signal density in the 10× Octapeptide treatment indicated a dose-responsive increase in the production of high MW HA in dermal fibroblasts.

### Example 3. Multi-Center Evaluation of Topical Hyaluronic Acid and Octapeptide Formulation on Facial Skin

This example involved a study on the progressive effects of treatment using a topical HA and octapeptide formulation as part of a facial skin care routine in a third experiment. The formulation tested included topical application of the composition comprising high MW HA and octapeptide, SPF 30+, an optional ultra-light moisturizer post Week 4, and regular skin care using a gentle cleanser in a third experiment. This study evaluated the efficacy and safety of the composition comprising high MW HA and octapeptide in facial skin. Photographic analysis and histological analysis of biopsies using the treatment formulation, a topical application of the composition comprising high MW HA and octapeptide, SPF 30+, an optional ultra-light moisturizer post Week 4, also constituted an aspect of the third experiment.

### Material and Methods

This open label clinical study occurred over 4 months from November 2021-March 2022 and was approved by the Institutional Review Board, Advarra, Inc. (Columbia, MD). Eligible participants were healthy men and women ages 25-70, without clinically significant unstable medical conditions, that were willing to only use the topical study product and refrain from extended periods of sun exposure, topical treatments and procedures during the course of study participation. Exclusions to study participation included; a previous hypersensitivity or known allergy to any of the ingredients in the study product, use of retinols or topicals with actives within 30 days, injectable toxins or resurfacing procedures within 3 months, injectable fillers and oral isotretinoin within 6 months. Additionally, pregnancy, breastfeeding as well as participants planning on becoming pregnant during the study duration, were excluded.

Eligible, enrolled participants were given the topical serum, to apply twice daily and return to the office for follow-up visits at weeks 2, 4, and 8. All participants were supplied with a gentle cleanser for AM and PM use and an SPF 30+ to use in the AM. At every visit, participants cleansed their facial skin and acclimated for 15 minutes prior to assessments, photography and measurements. Participants then applied the topical serum and acclimated for 15 minutes prior to the second set of assessments, photography and measurements.

Skin Hydration Sensor Measurements. At every visit, participants had 3 measurements on the right and left sides of the face using a skin hydration sensor measurement system by Wearifi (Chicago, IL). 15 minutes post cleansing the facial skin and 15 minutes post application of the topical study product. Measurements were performed in the same area at every visit. The measurement depth of the sensor is optimized to 50 μm, capturing the water content across stratum corneum and upper epidermis. The measurement is a quantitative result of volumetric ratio of water in skin tissue, which represents an absolute value with direct relevance to the effective hydration.

Participant Assessments and Satisfaction. At all follow-up visits, post 15 minutes of cleansing the skin, participants completed an assessment of their facial skin (using a 5-point scale) and overall satisfaction with the topical study product (using a 7-point scale) compared to baseline. And at every visit, post 15 minutes of applying the topical serum, participants completed an assessment of their facial skin (using a 5-point scale).

Investigator Assessments. At baseline and week 8, investigators completed an assessment of the facial skin, 15 minutes after cleansing. A 10-point scale was used to assess fine lines/wrinkling, crepiness, texture, erythema, dryness and moisture/hydration. Scale: 0 absent; 1-3 mild, 4-6 moderate, 7-9 severe.

Photography. At every visit, photos were taken 15 minutes post cleansing the skin and post application of the study product. There were 3 different camera systems used in this study. VISIA® Skin Analysis System, (Canfield Scientific, Inc., Parsippany, NJ), LifeViz® Infinity and Life Viz® Micro (Quantificare, Inc. US, Cumming, GA).

Biopsies. A 3 mm punch biopsy was performed periauricular at baseline, and post application at week 2, week 4 and week 8. Participants that elected to biopsies were instructed to apply the topical serum periauricular twice a day for the duration of the study. An independent dermatopathologist, evaluated the tissue pre and post application of the study product.

Histology. Biopsy samples were fixed and sectioned for histological analysis according to standard protocols. Sectioned biopsy samples were mounted on glass slides and stained with hematoxylin and cosin to reveal epidermal and dermal cellular structure, including the structure of the dermal ECM. Sectioned biopsy samples were mounted on glass slides and labeled for CD44 protein expression using an anti-CD44 antibody via immunohistochemistry following standard protocols. CD44 labeling is indicated on the sections in a darker brown staining.

### Results

An independent statistician completed the analyses using descriptive statistics, parametric and non-parametric tests.

Mean Hydration Levels on Clean Skin Increase Progressively with Continued Treatment.

Analyses were completed on clean skin between baseline and the follow-up visits and between clean skin and post application of the study product at each visit. A pairwise comparison between each timepoint was also computed. 15 minutes post cleansing facial skin: At each follow-up visit there was an increase in the hydration measurements compared to baseline. At Week 4 and Week 8 there was a statistically significant increase in hydration compared to baseline and the prior visit (FIG. 7).

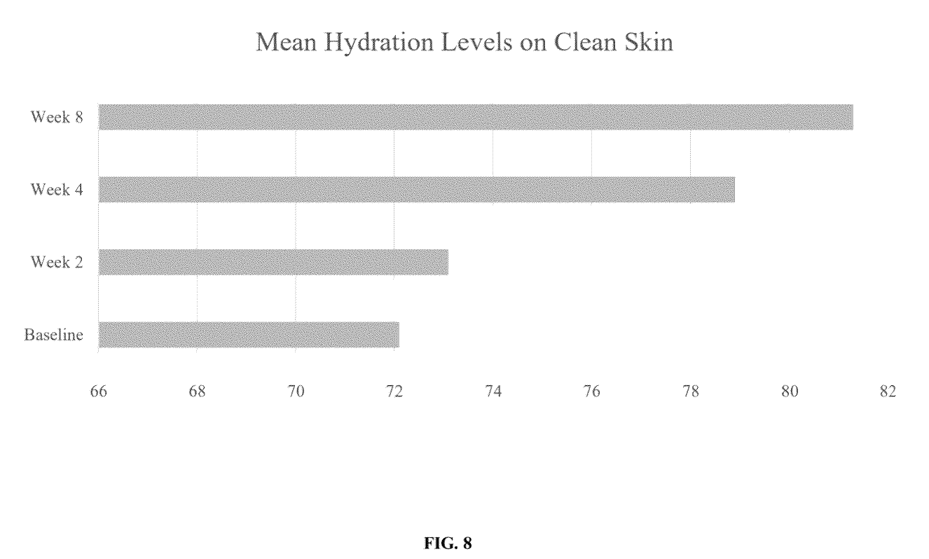

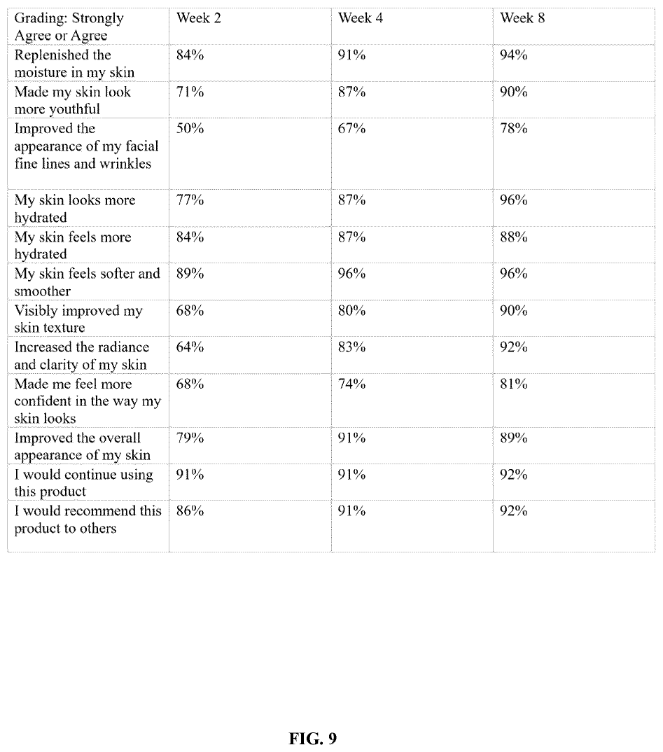

At every visit, hydration measurements were statistically significant higher as measured in post application compared to clean skin (FIG. 8). At weeks 4 and 8 post application measurements were also significantly higher than post application measurements at baseline and week 2.

### Compilation of Participant Assessment and Satisfaction Post-Cleanse and Post-Application

15 minutes post cleansing the skin, at weeks 2, 4 and 8, participants that gave a favorable rating, either strongly agree or agree, were statistically significant compared to week 2 for the following assessments (FIG. 9).

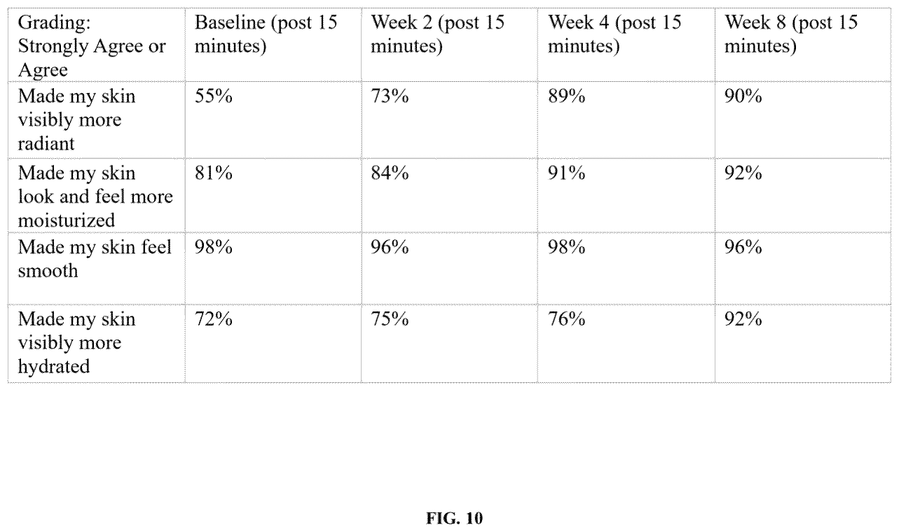

15 minutes post application of the study product at all visits, the percentage of participants that rated favorably continued to increase at each visit. (FIG. 10).

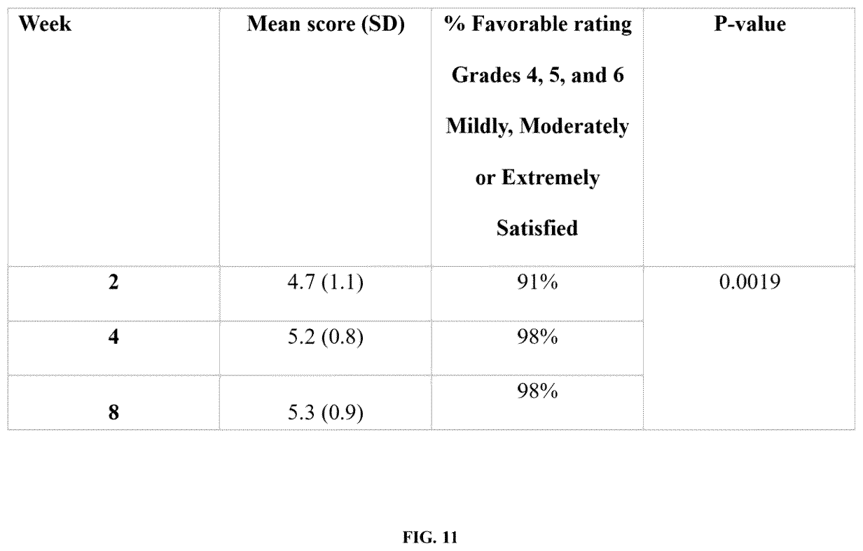

As seen in FIG. 11, participant overall satisfaction was rated favorably, 4-6 on the scale, and statistically significant: 90.0% at week 2, increased to 97.8% at week 4, and 97.9% at week 8. (p=. 0061)

### Results of Investigator Assessments

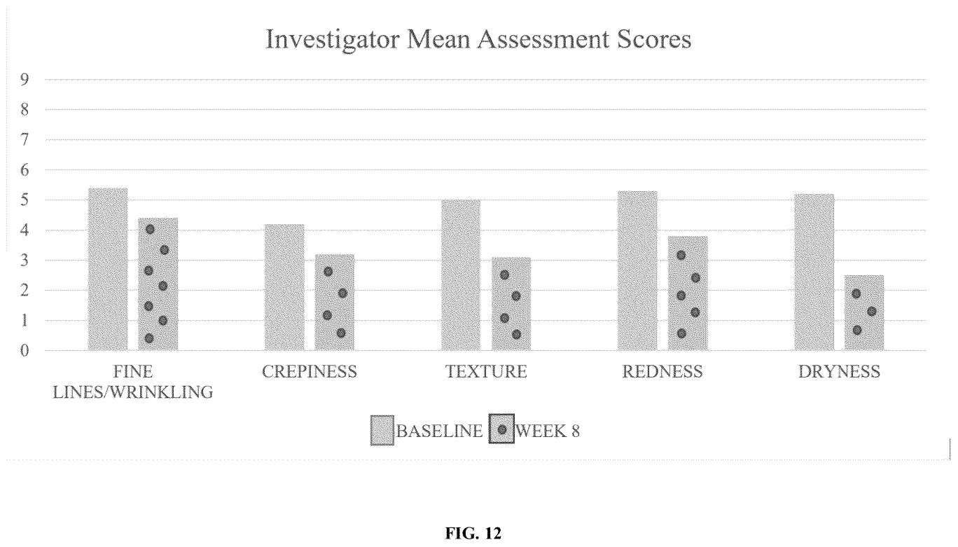

Mean assessment scores by the investigator were compared in five categories of features related to facial skin health and appearance (FIG. 12). Compared to baseline all assessment grades were statistically significant p<. 0001 at the Week 8 mark. Grades for decreased fine lines/wrinkling, crepiness, texture, erythema, and dryness decreased, and moisture/hydration increased.

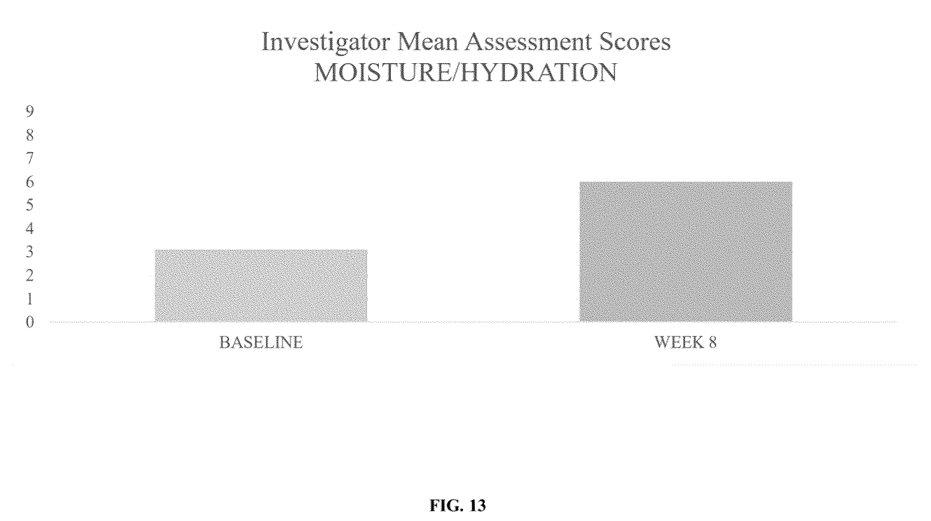

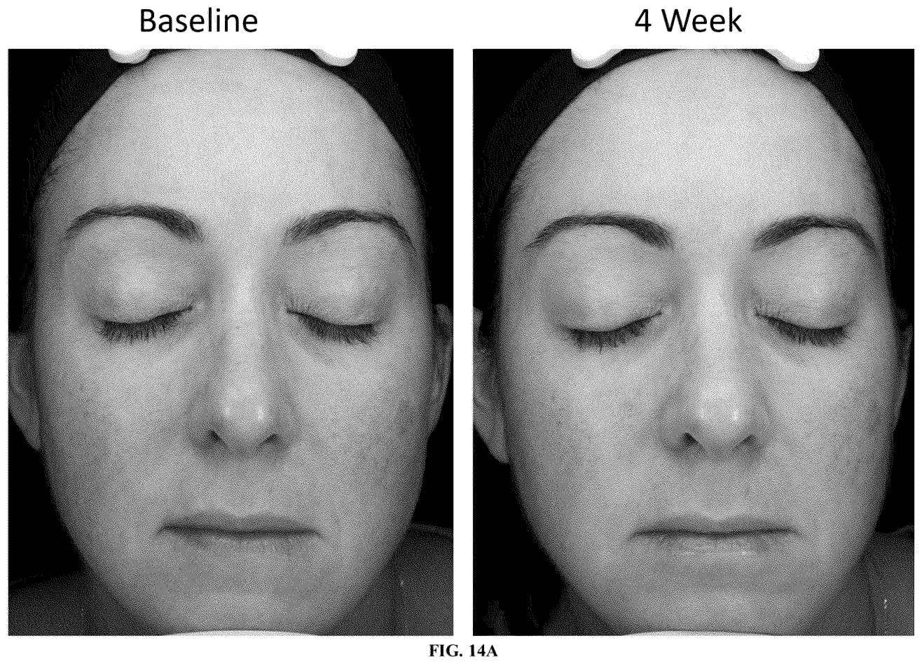

Mean assessment scores by the investigator were compared across the study at baseline and at Week 8 after initiation of treatment. As demonstrated in FIG. 13, mean assessment of facial skin moisture/hydration increased extensively.

Photographs and Analysis: Treatment with a Formulation of HA Immerse Serum (High MW HA+Octapeptide), SPF 30+, an Optional Ultra-light Moisturizer Post-Week 4.

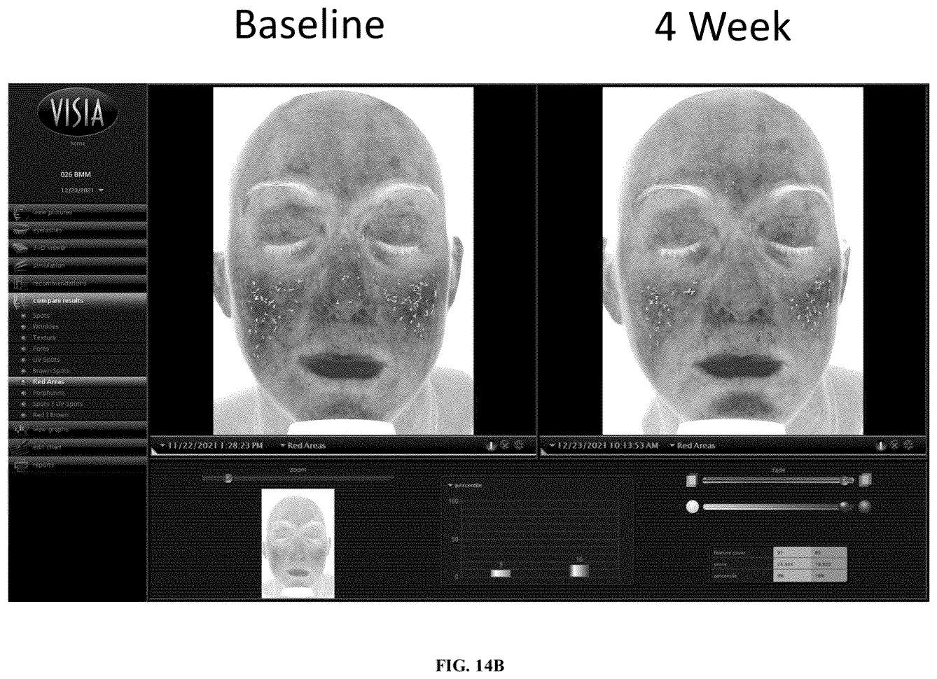

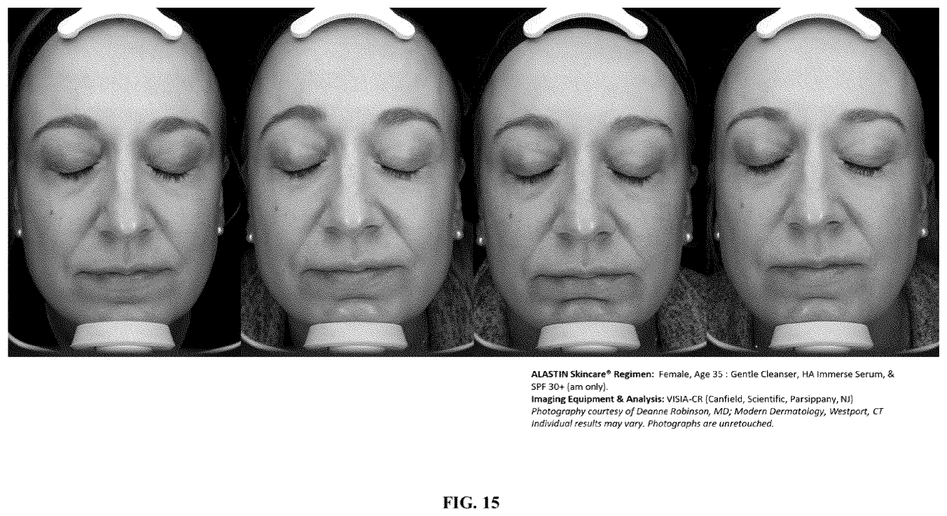

FIG. 14-21 are photographs, analysis and histology sections from subjects at baseline and at various time points following initiation of treatment with the composition comprising high MW HA and octapeptide, SPF 30+, an optional ultra-light moisturizer post Week 4, and regular skin care using a gentle cleanser as part of a third experiment.

FIG. 14A demonstrates improved appearance and decreased redness after Week 4 in a female subject, age 39.

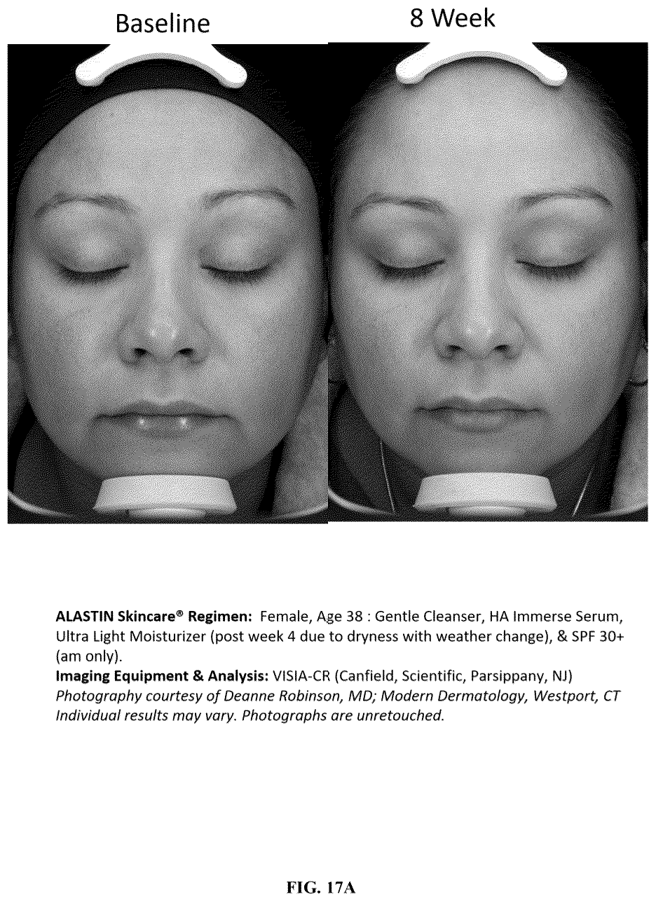

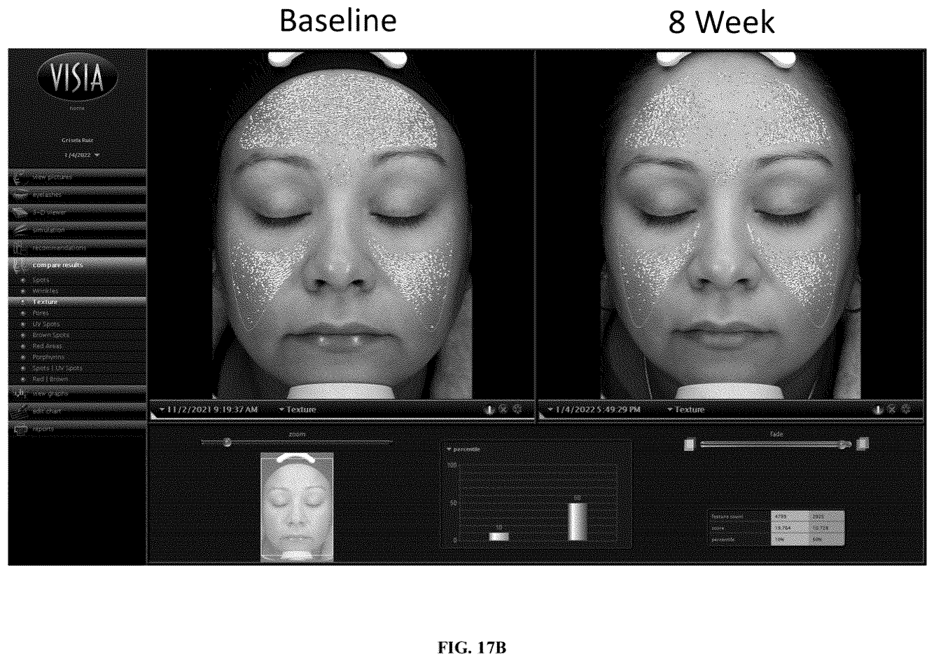

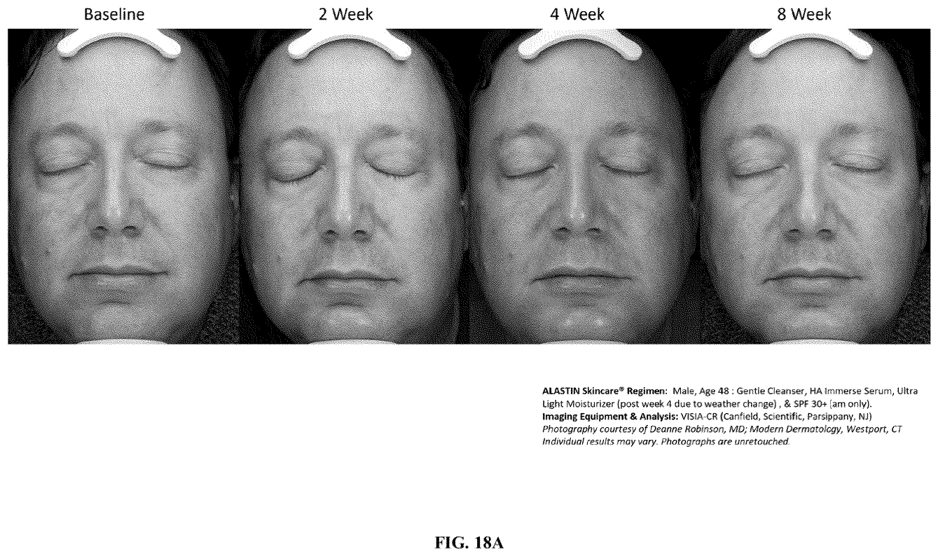

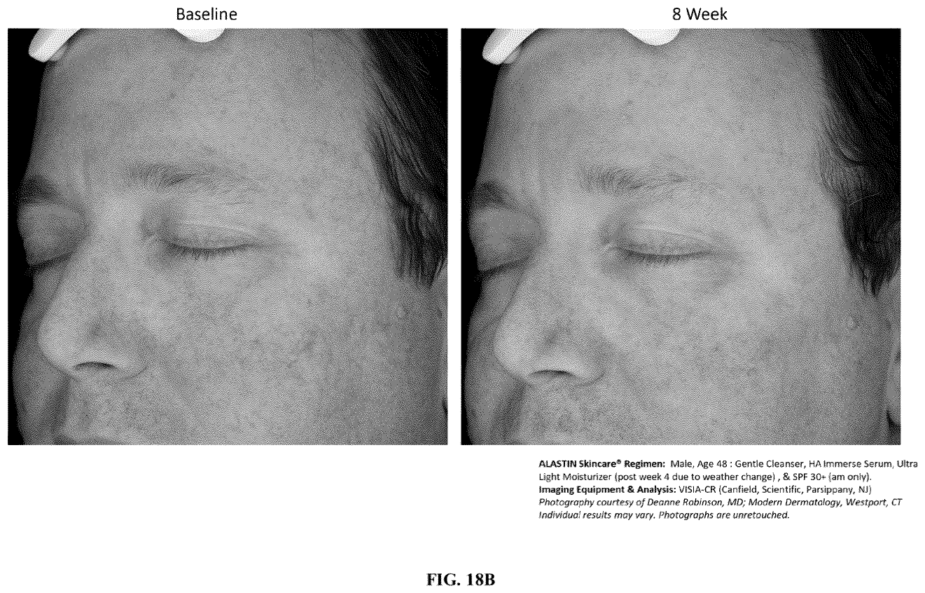

FIG. 14B depicts results of VISIA® Skin Analysis System on the subject from FIG. 17A demonstrating a quantifiable reduction in erythema.

FIG. 15 shows photographs taken (from left to right) at baseline, at Week 2, at Week 4, and at Week 8 following treatment to demonstrate improvements to progressive effects of skin improvement in a female subject, age 35. Visible, progressive changes to improve the appearance of fine lines, skin texture and redness are evident when comparing each time point indicating that continued treatment yields progressively enhanced results.

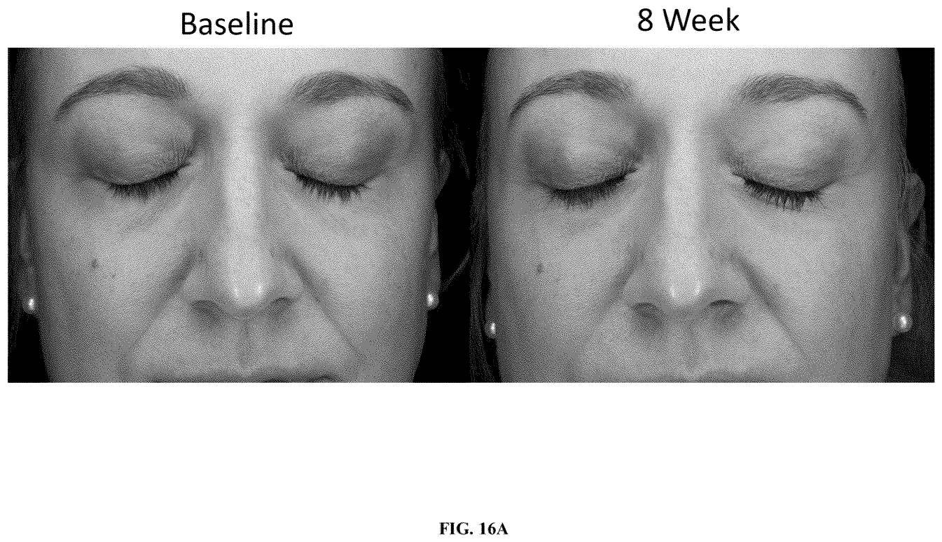

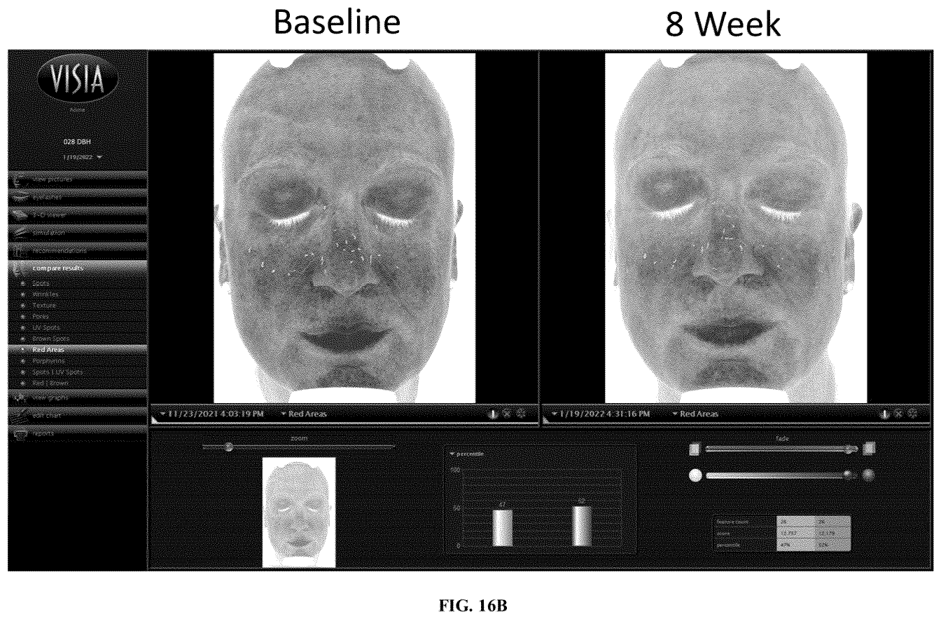

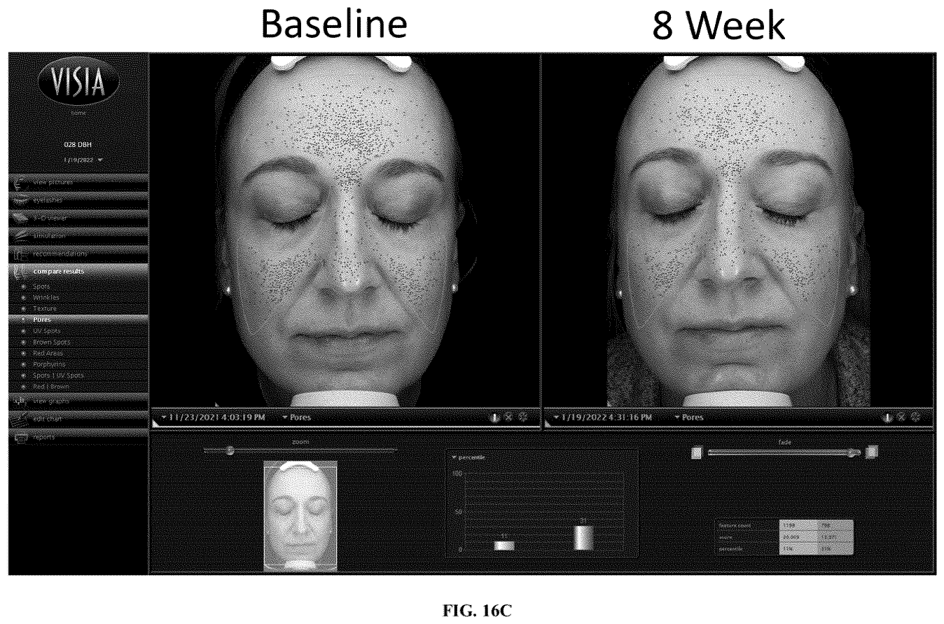

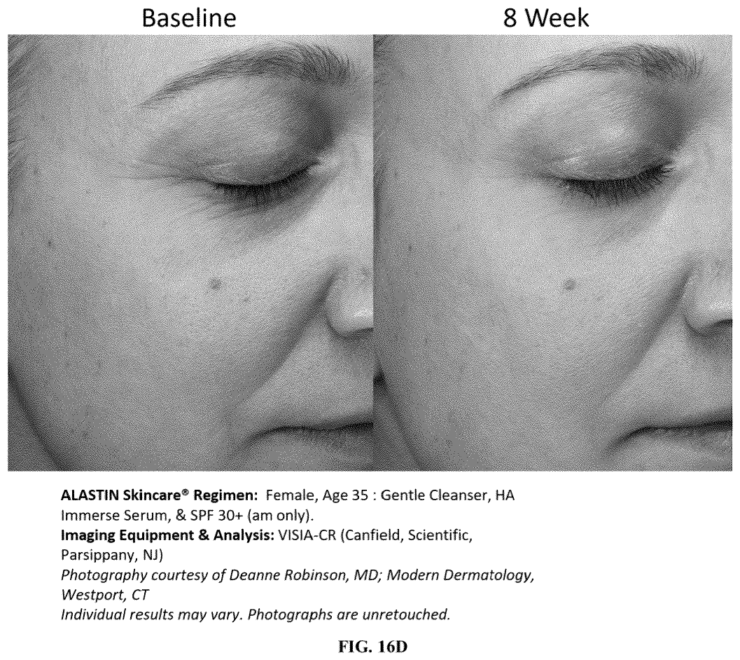

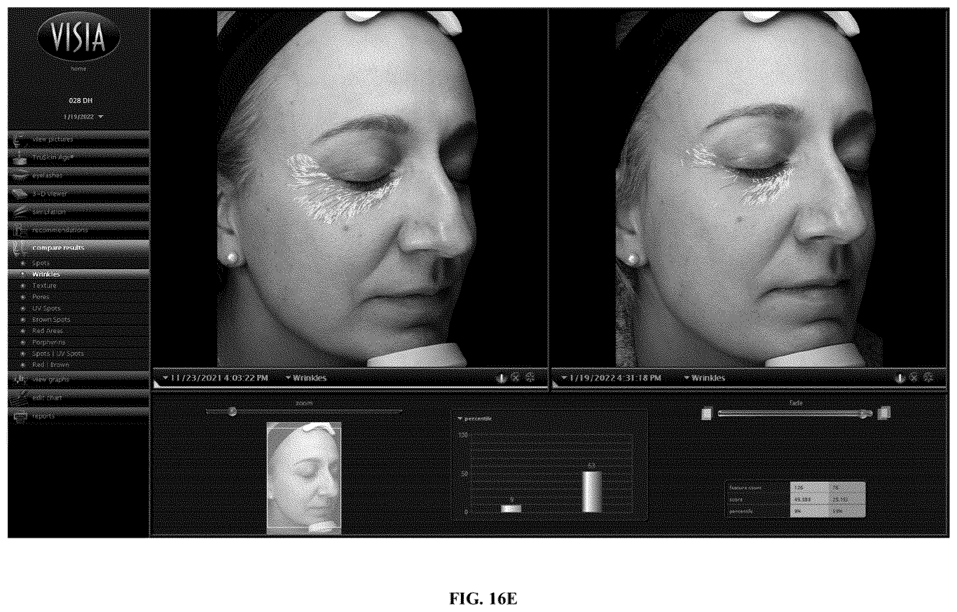

FIGS. 16A-16C demonstrate the extent of improvement in quantifiable features of appearance in the subject from FIG. 15 comparing baseline and Week 8 measurements. FIG. 16B shows the results of the VISIA® Skin Analysis System to indicate a measurable reduction in red areas of the face. FIG. 16C quantifies the effect of treatment using the VISIA® Skin Analysis System to lead to a reduction total extent of pore size area on the face of the subject.

FIGS. 16D-16E demonstrate the extent of the decreased appearance in fine lines and wrinkles after 8 weeks of treatment. Particularly prominent is the decrease in appearance of fine lines and wrinkles in skin around the eyes. FIG. 16E quantifies the effect of treatment using the VISIA® Skin Analysis System to lead to a reduction the visibility of fine lines and wrinkles on the face of the subject.

FIGS. 17A-17B show photographs and analysis at baseline and at Week 8 following treatment to demonstrate improvements to the appearance of skin texture in a female subject, age 38. FIG. 17B quantifies the effect of treatment using the VISIA® Skin Analysis System to lead to an improved appearance of skin texture on the face of the subject.

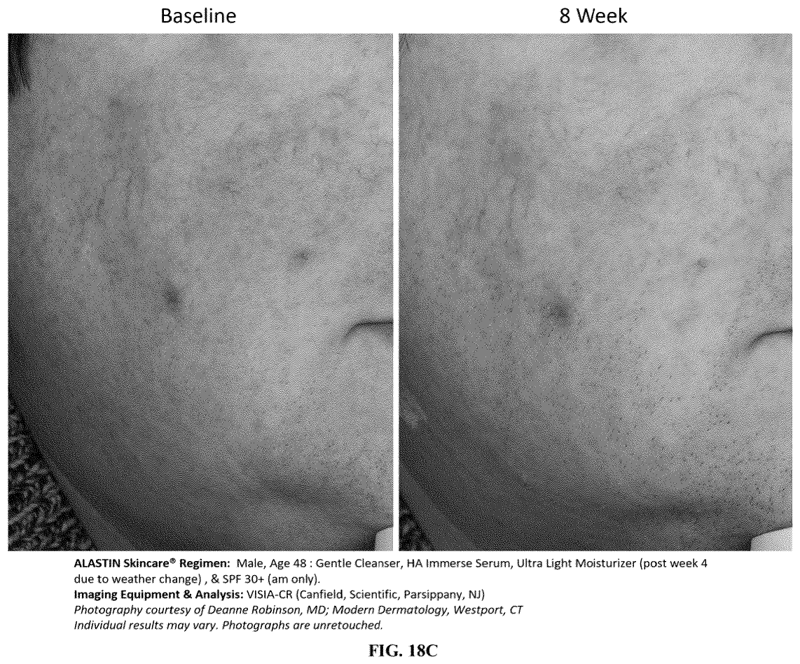

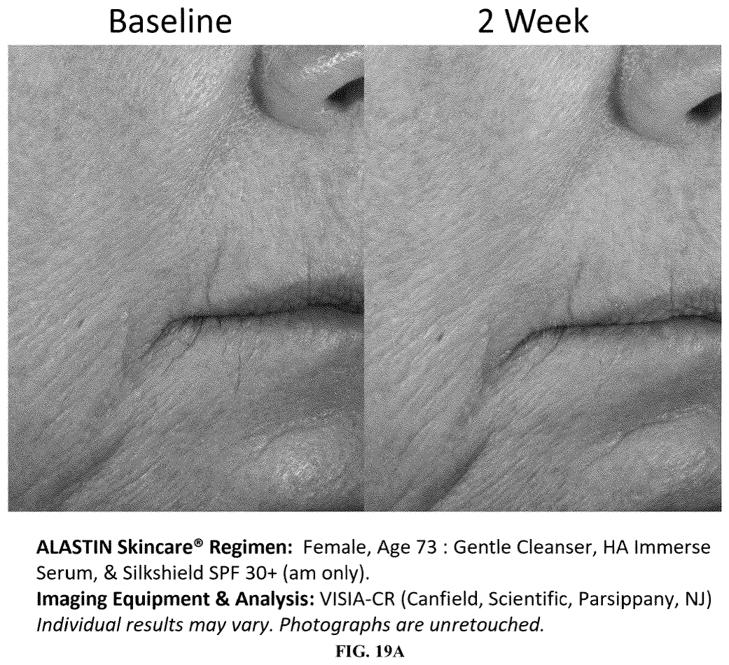

FIGS. 18A-18C show photographs demonstrating the effects of treatment to show a noticeable reduction in the appearance of erythema and rosacea in a male subject, age 48.

FIG. 18A shows the progressive effects of treatment when comparing baseline to Week 2, Week 4, and Week 8 in visible redness reduction in a male subject, age 48.

FIGS. 18B-18C show in the subject from FIG. 18A close-up views to illustrate the extent of visible reduction in erythema and rosacea in both the left and right check areas at Week 8. Particularly evident is the reduction in visibility of fine blood vessel following this treatment period.

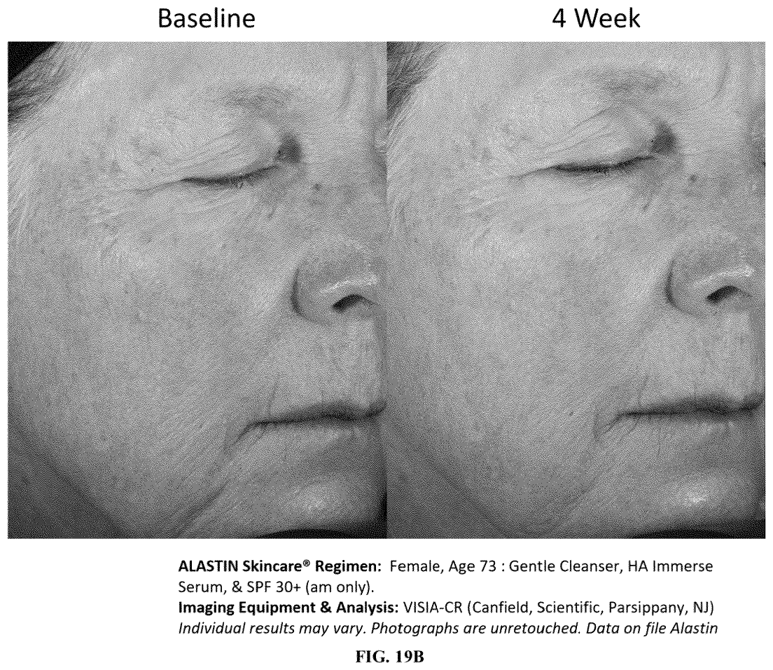

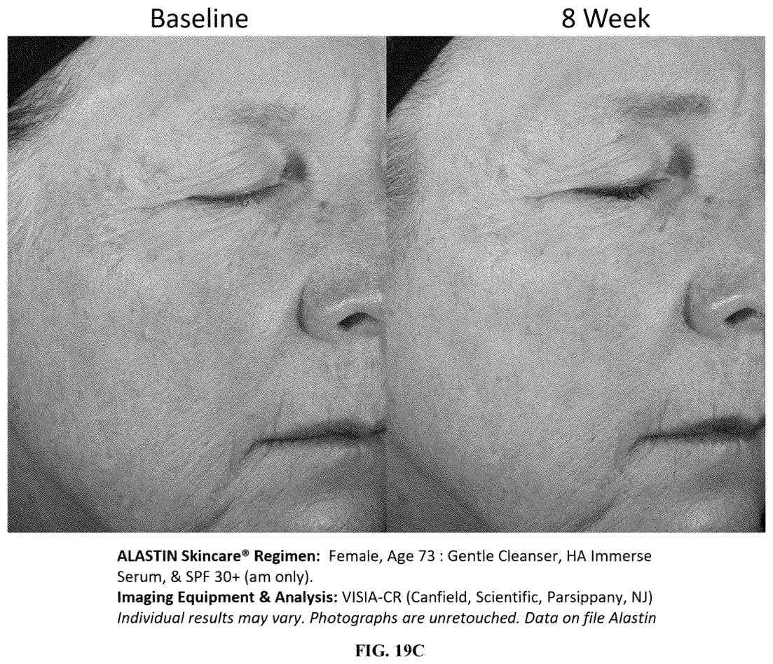

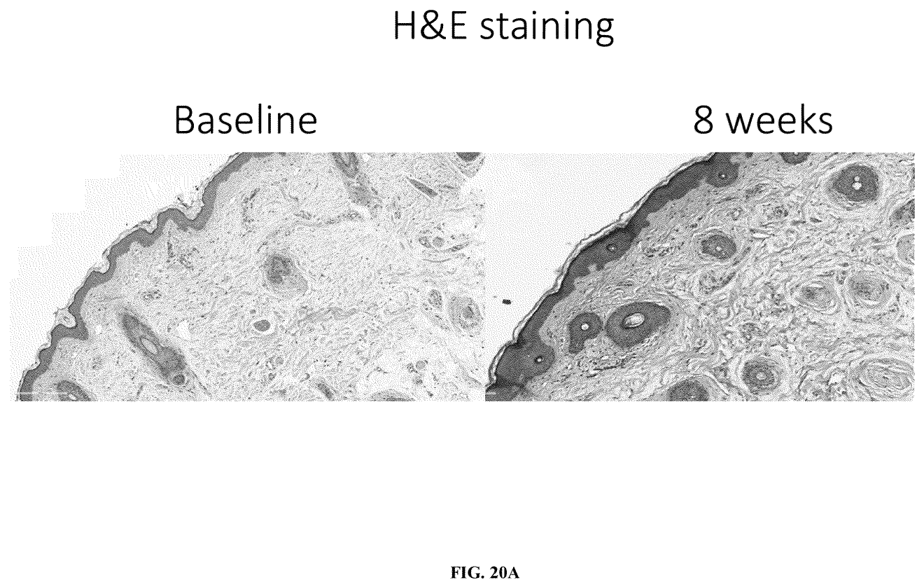

FIGS. 19A-19C show the progressive effects of reducing the visibility of deep wrinkles near the mouth, fine lines throughout the face and a decrease in the appearance of skin crepiness yielding a smoother and tauter skin appearance in a female subject, age 73 when comparing baseline, Week 2, Week 4 and Week 8.

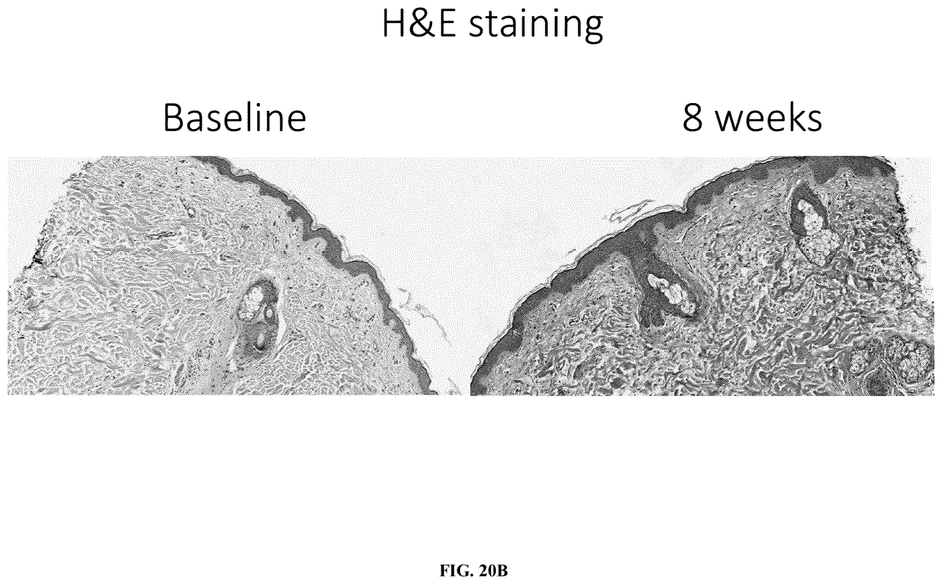

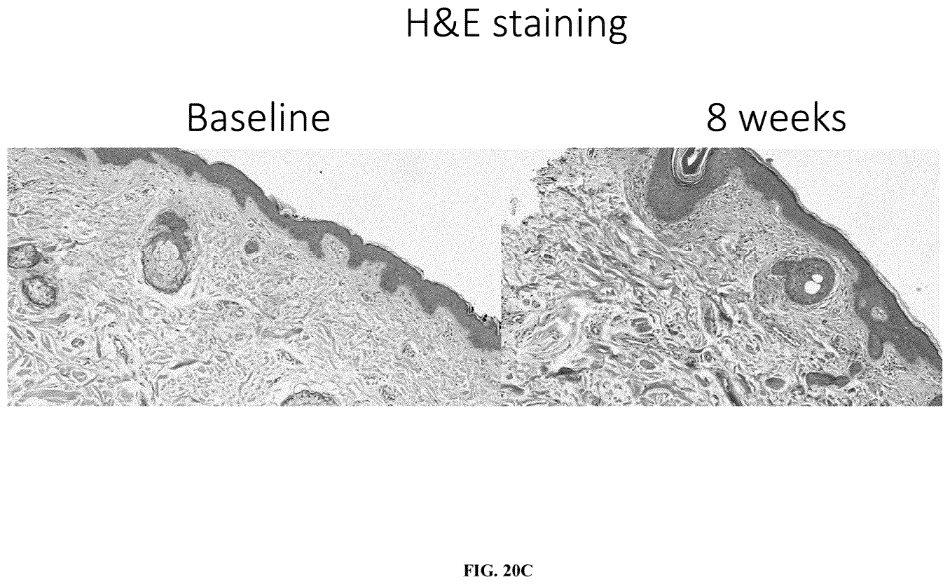

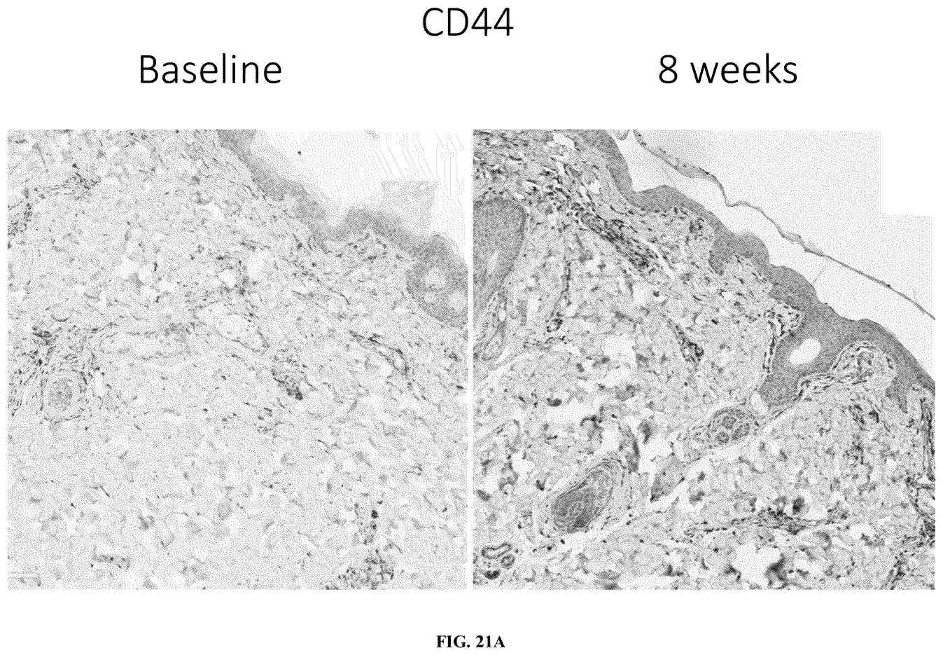

FIGS. 20A-20C show a histological analysis of periauricular biopsies of three subjects at baseline and at Week 8 following treatment with HA Immerse Serum (high MW HA+octapeptide). FIG. 20A-20C each show hematoxylin and cosin-stained sections from biopsies from the same subject compared at baseline and at Week 8 (3 subjects analyzed in total). Evident in each baseline sample is the extent of dermal solar elastosis visible as old, thin collagen fibers in the ECM. Following 8 weeks of use of HA Immerse, the old, thin collagen fibers in the dermis have been replaced with thicker healthier collagen fibers producing a much more dense and healthier dermis and ECM. This dramatic reversal of the cellular, extracellular and structural features of solar elastosis was an unexpected and significant finding after only 8 weeks of treatment in all three subjects analyzed. The extent of recycling of the old ECM and replacement with a dense, healthier one indicates the therapeutic benefit of treatment in both superficial and deep regions of the dermis.

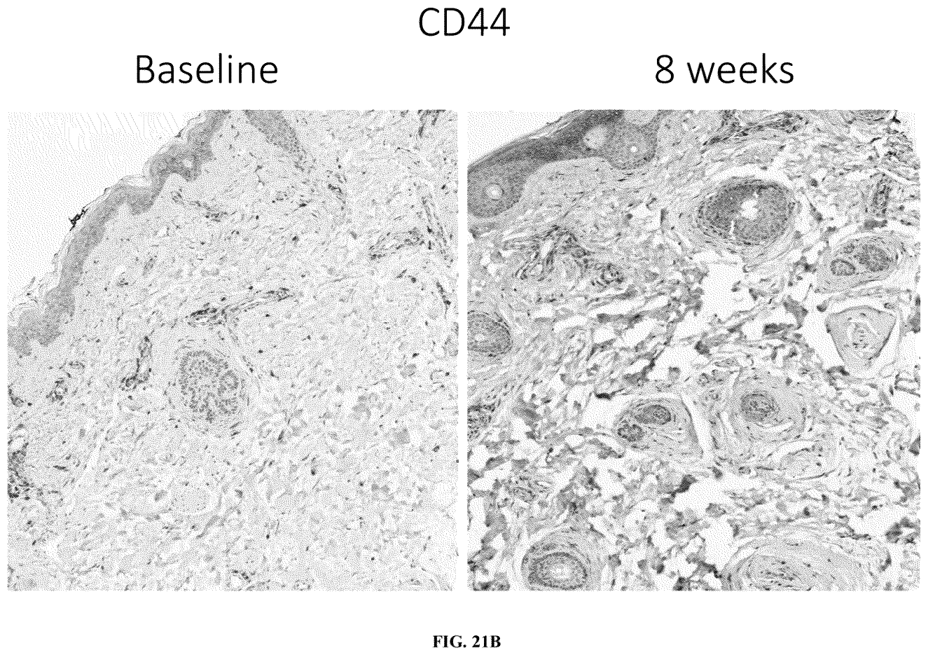

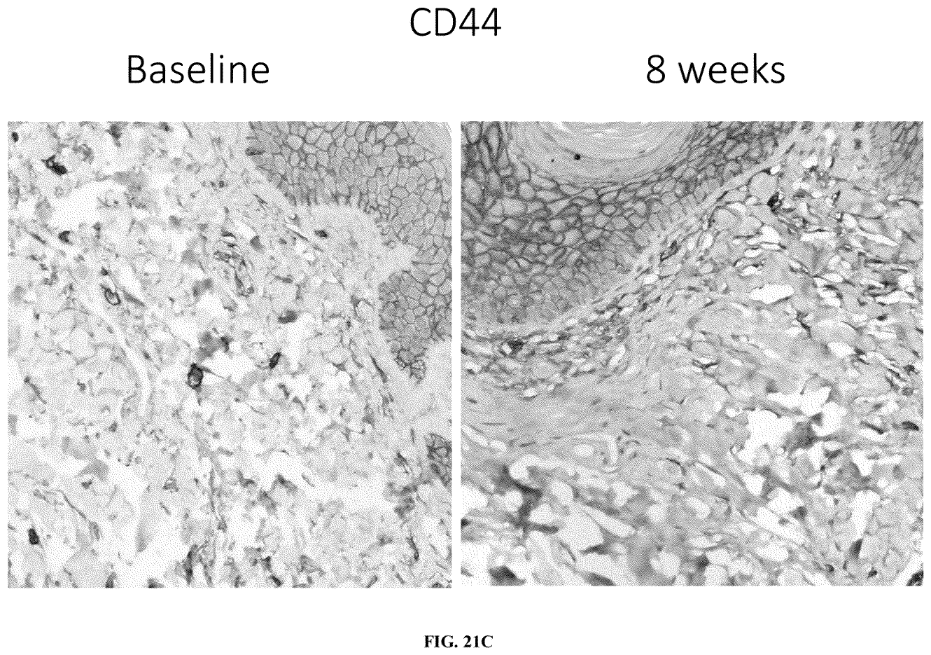

FIGS. 21A-21C show as histological analysis of CD44 staining within periauricular biopsies of two subjects at baseline and at Week 8 following treatment with HA Immerse Serum (high MW HA+octapeptide). FIG. 21A shows sections from a first subject with CD44 expression labeled in brown via immunohistochemistry. As CD44 is a receptor for HA and has been shown to be upregulated upon increased extracellular HA, elevated CD44 labeling in both epidermis and dermis at Week 8 indicates increased stimulation and secretion of HA. FIGS. 21B-21C show sections from a second subject with CD44 expression labeled in brown via immunohistochemistry. Again, elevated CD44 expression at Week 8 indicates increased stimulation and secretion of HA. Elevated CD44 expression is seen throughout the epidermis and dermis.

FIG. 22 shows the chemical structure of octapeptide-45, comprising an amino acid sequence Gly-Pro-His-Gly-Val-Arg-Glu-Ala. octapeptide-45 is the octapeptide included in the formulation of HA Immerse Serum.

### Example 4. Exemplary Formulations

Exemplary embodiments of compositions described herein are provides in Tables 2 and 3 of a weight concentration of a solution as expressed as % (w/w).

TABLE 2
Exemplary Composition

| Ingredient | % w/w |
|---|---|
| Water/Aqua/Eau | 50-99 |
| Glycerin | 1-5 |
| Polyacrylate-13 | 0.5-5 |
| Sodium Hyaluronate | 0.001-0.1 |
| Sodium Hyaluronate Crosspolymer | 0.0001-0.01 |
| Octapeptide | 0.0001-0.01 |
| Hexapeptide-11 | 0.0001-0.01 |
| Tremella fuciformis Sporocarp Extract | 0.0001-0.1 |
| Lactoferrin | 0.01-1 |
| Tetradecyl Aminobutyroylvalylaminobutyric Urea | 0.001-2 |
| Trifluoroacetate | |
| Xylitol | 0.001-2 |
| Propanediol | 0.001-2 |
| Anhydroxylitol | 0.001-2 |
| Phospholipids, | 0.001-2 |
| Xylitylglucoside | 0.001-2 |
| Phosphatidylserine | 0.001-2 |
| Glucose | 0.001-2 |
| Hydroxymethoxyphenyl Decanone | 0.001-2 |
| Tocopherol | 0.001-2 |
| Ascorbyl Palmitate | 0.001-2 |
| Dimethicone | 0.001-2 |
| Caprylyl Methicone | 0.001-2 |
| Caprylic/Capric Triglyceride | 0.001-2 |
| Polyisobutene | 0.001-2 |
| Caprylyl Glycol | 0.001-2 |
| Disodium EDTA | 0.001-2 |
| Betaine | 0.001-2 |
| Polysorbate 20 | 0.001-2 |
| Ethylhexylglycerin | 0.001-2 |
| Caprylhydroxamic Acid | 0.001-2 |
| Sorbitan Isostearate, | 0.001-2 |
| Helianthus Annuus (Sunflower) Seed Oil | 0.001-2 |
| Lecithin | 0.001-2 |
| Pentylene Glycol | 0.001-2 |
| Magnesium Chloride | 0.001-2 |
| Phenoxyethanol | 0.001-2 |
| Potassium Sorbate | 0.001-2 |

TABLE 3
Exemplary Composition

| Ingredients | % w/w |
|---|---|
| Water/Aqua/Eau | 85-99 |
| Glycerin | 1.0-8.0 |
| Polyacrylate-13 | 0.1-4.0 |
| Sodium Hyaluronate | 0.0001-0.03 |
| Sodium Hyaluronate Crosspolymer | 0.0001-0.03 |
| Octapeptide | 0.0001-0.01 |
| Hexapeptide-11 | 0.0001-0.01 |
| Tremella fuciformis Sporocarp Extract | 0.001-0.09 |
| Lactoferrin | 0.001-0.15 |

### Example 5. Gene Expression Studies for Compositions Comprising TriHex+Octapeptide Methods

Human adult dermal fibroblast cell lines were cultured in media from ZenBio, (Durham, NC) and maintained at 37° C. in a 5% CO₂ incubator in 48-well plates. After 48 hours, the cells were treated with the compounds listed in Table 4 below. One group was left untreated for comparison purposes. After 24 hours, the cell media was removed, and the cells were washed 1× with PBS. The cells were lysed, and the lysates were generated using 100 μl of RNA lysis buffer (“10×RNA lysis buffer”, Takara Bio, Cat. Num. 635013), diluted to 1× in RNAse-free microcentrifuge tubes. RNA extraction, library construction, and sequencing to 25M paired-end 100 bp reads per sample were then performed. Differentially expressed genes were identified, and pathway enrichment was assessed using the Reactome Pathway database.

TABLE 4
Compositions for In Vivo Gene Expression Studies

| Compound | Concentration |
|---|---|
| Tripeptide 1 + Hexapeptide-12 (TriHex) | 20 μg/ml of each peptide |
| | (40 μg/ml total) |
| Octapeptide-45 | 20 μg/ml |
| Tripeptide 1 + Hexapeptide-12 (TriHex) + | 20 μg/ml of each peptide |
| Octapeptide-45 | (60 μg/ml total) |
| No Treatment | — |

### Results

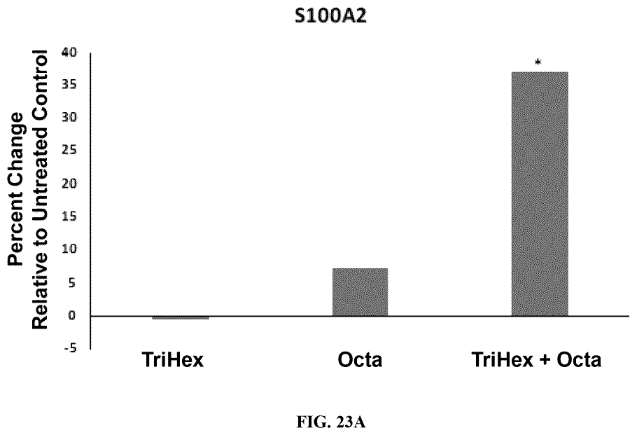

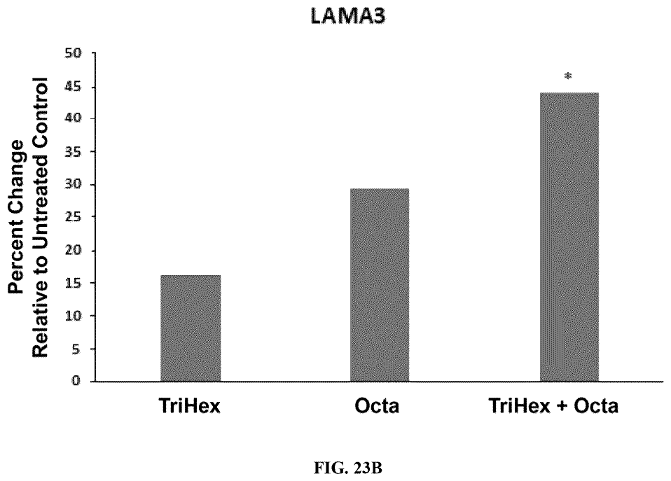

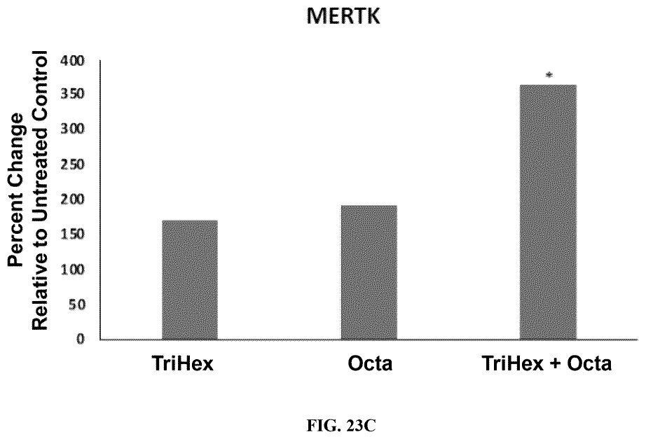

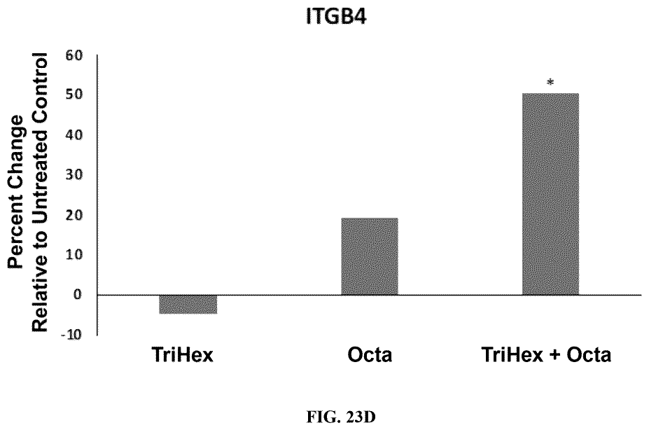

As shown in FIGS. 23A-D, the combination of TriHex+octapeptide-45 exhibited gene expression synergy for the S100A2, LAMA3, MERTK, and ITGB4 genes. S100A2 is S100 calcium binding protein A2. It is a secreted matrisome-associated protein that is expressed in the dermis. S100A2 activates protein phosphatase 5 (PP5) in a calcium-dependent manner. PP5 is a serine/threonine phosphatase involved in oxidative stress responses. LAMA3 is laminin, alpha 3. LAMA3 is the gene that encodes the alpha-3 chain of laminin 5, which is a major link between the epidermal basal cells and the papillary dermis. It initiates hemidesmosome formation and provides stable attachment of the epidermis to the dermis. Laminin 5 also accelerates the assembly of basement membranes and may enhance the recovery of damaged skin. MERTK is tyrosine-protein kinase MER. MERTK tranduces signals from the extracellular matrix into the cytoplasm by binding to several ligands including LGALS3, TUB, TULP1, or GAS6. Following activation by ligand, MERTK plays a role in various processes such as macrophage clearance of apoptotic cells, platelet aggregation, cytoskeleton reorganization, and engulfment. ITGB4 is integrin beta 4. ITGB4, partnered with integrin alpha 6, is the receptor for laminin. The a6ß4 integrin protein is particularly important in strengthening and stabilizing the skin because it is a component of hemidesmosomes in the epidermis.

Table 5 lists multiple pathways related to ECM regulation for which gene expression is upregulated by octapeptide-45 or the combination of TriHex+octapeptide-45, indicating therapeutic benefit in ECM regulation, including elastin synthesis. The p-value is the result of an over-representation analysis. The over-representation analysis is a statistical (hypergeometric distribution) test that determines whether certain Reactome pathways are over-represented (enriched) in the data. This test produces a probability score, which is corrected for false discovery rate using the Benjamani-Hochberg method.

TABLE 5
Genes are enriched for pathways related to ECM regulation.

| Pathway | | Entities |
|---|---|---|
| Identifier | Pathway Name | pValue |
| R-HSA-446107 | Type I hemidesmosome assembly | 9.76 × 10⁻⁷ |
| R-HSA-1474244 | Extracellular matrix organization | 1.61 × 10⁻⁵ |
| R-HSA-8939247 | RUNX1 regulates transcription of genes | 6.86 × 10⁻⁵ |
| | involved in interleukin signaling | |
| R-HSA-2022090 | Assembly of collagen fibrils and other | 2.07 × 10⁻⁴ |
| | multimeric structures | |
| R-HSA-1912408 | Pre-NOTCH transcription and | 4.73 × 10⁻⁴ |
| | translation | |
| R-HSA-446728 | Cell junction organization | 5.72 × 10⁻⁴ |
| R-HSA-1474290 | Collagen formation | 7.43 × 10⁻⁴ |
| R-HSA-2022870 | Chondroitin sulfate biosynthesis | 8.58 × 10⁻⁴ |
| R-HSA-1912422 | Pre-NOTCH expression and processing | 9.67 × 10⁻⁴ |
| R-HSA-3000157 | Laminin interactions | 1.31 × 10⁻³ |
| R-HSA-1500931 | Cell-cell communication | 1.54 × 10⁻³ |
| R-HSA-1474228 | Degradation of extracellular matrix | 2.04 × 10⁻³ |
| R-HSA-3000171 | Non-integrin membrane-ECM | 4.92 × 10⁻³ |
| | interactions | |
| R-HSA-1793185 | Chondroitin sulfate/dermatan sulfate | 6.96 × 10⁻³ |
| | metabolism | |
| R-HSA-3000178 | ECM proteoglycans | 8.10 × 10⁻³ |

### Example 6. Ex Vivo Study of MFAP4 and Tropoelastin (TE) Expression for Skin Samples Treated with TriHex+Octapeptide

### Methods

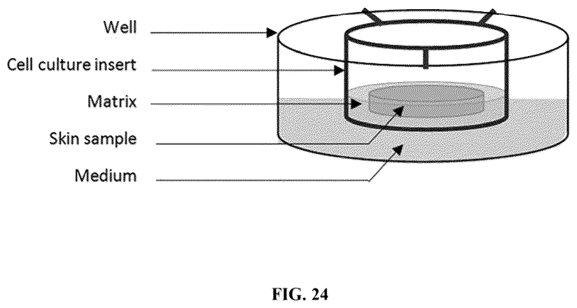

Referring to FIG. 24, human skin tissue was defatted and cut into a diameter of approximately 7 mm and placed in a transwell insert with cell culture media (DMEM/F12 containing 1.88 μM CaCl₂), 2% fetal bovine serum, 50 μM adenine, 0.02 nM tri-iodothyronine, 1% ITS-X (insulin, transferrin, selenium mix), 1% glutagro (a glutamine equivalent), 1% pen/strep, and 1% gentamycin). The medium was changed daily. The human skin tissue samples were equilibrated for 3 days, then treated with the compounds noted in Table 6 once daily for 7 days. After 7 days of treatment, the skin samples were fixed in formalin and embedded in paraffin following industry standard procedures. The resulting blocks were prepared and sectioned for immunofluorescent staining.

Antibodies directed to tropoelastin (TE) (available from Elastin, Owensville, MO) and antibodies against microfibrillar-associated protein 4 (MFAP4) (available from ThermoFisher Waltham, MA) were used to detect the production of new elastin in the treated cells. The appropriate fluorescent secondary antibodies were used for detection by fluorescence microscopy. Images were obtained and analyzed using ImageJ software.

Data was obtained from a minimum of three donor skin samples and replicates or triplicates were performed for each compound treated.

TABLE 6
Compositions for Ex Vivo Studies

| Compound | Concentration |
|---|---|
| Tripeptide 1 + Hexapeptide-12 (TriHex) | 20 μg/ml of each peptide |
| | (40 μg/ml total) |
| Octapeptide-45 | 20 μg/ml |
| Tripeptide 1 + Hexapeptide-12 (TriHex) + | 20 μg/ml of each peptide |
| Octapeptide-45 | (60 μg/ml total) |
| No Treatment | — |

### Results

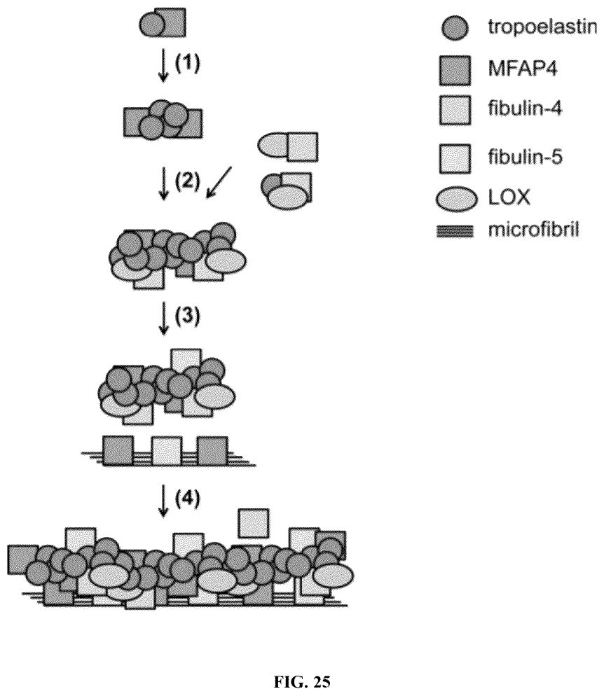

Microfibrillar-associated protein 4 (MFAP4) is an extracellular glycoprotein found in elastic fibers. MFAP4 actively promotes tropoelastin (TE) self-assembly and is a ligand of microfibrils and tropoelastin involved in proper elastic fiber organization. See P. Bartosz et al., 291 J. Biol. Chem. 1103-14 (January 2016) (FIG. 10) (reproduced as FIG. 25).

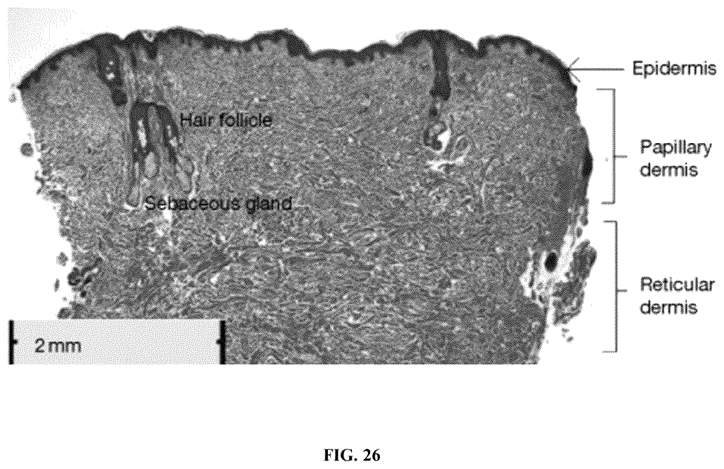

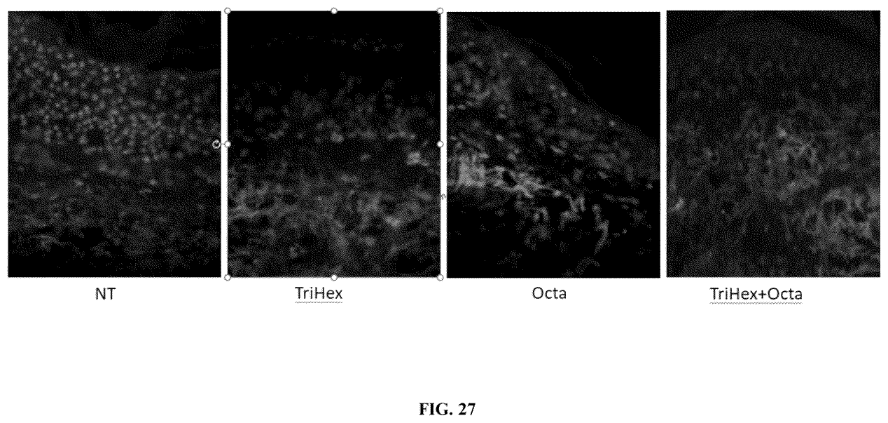

FIG. 27 is an image of immunofluorescent staining of cells with antibodies directed to MFAP4. MFAP4 expression increases with TriHex (second from left), octapeptide-45 (second from right), and TriHex+octapeptide-45 treatment (right). The increase in MFAP4 expression with TriHex+octapeptide-45 shows an invasion of new fibrils into the papillary dermis (see also FIG. 26) that is greater than that observed for TriHex and octapeptide-45 alone. The expression of MFAP4 suggests the opportunity for formation of new elastin fibers.

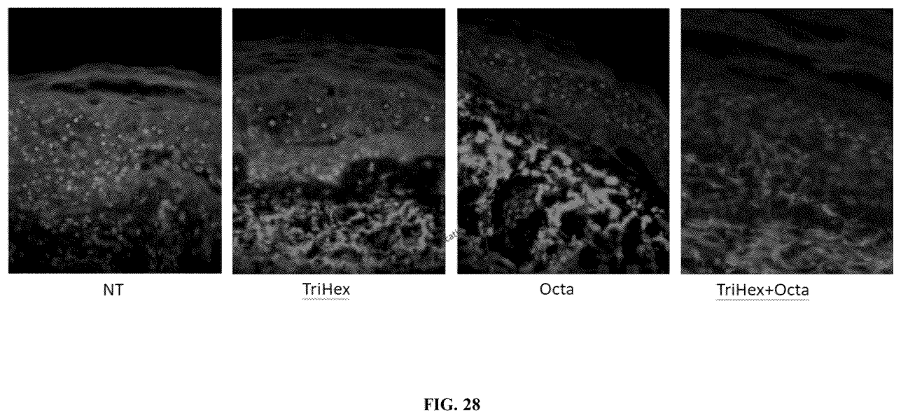

FIG. 28 is an image of immunofluorescent staining of cells with antibodies directed to TE. TE expression increases with TriHex, octapeptide-45, and TriHex+octapeptide-45. The increase in TE production with TriHex+octapeptide-45 shows an invasion of new elastin fibers into the papillary dermis that is greater than that observed after treatment with TriHex and octapeptide-45 alone. In addition, the alignment of the fibers is more organized with the combination treatment, suggesting that the newly formed elastin fibers are more stable.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A topical composition for stimulating synthesis of high MW HA, the composition comprising:
a synthetic, isolated, or recombinant polypeptide comprising an amino acid sequence GPHGVREA (SEQ ID NO:1), GDGDGASA (SEQ ID NO: 2), GPMGPSGP (SEQ ID NO: 3), GLGPGARA (SEQ ID NO: 4), GPQGFQGP (SEQ ID NO: 5), GPMGPRGP (SEQ ID NO: 6), or GPGKNGDD (SEQ ID NO: 7), wherein the polypeptide comprises no more than 20 amino acids;
a tripeptide-1; and
a hexapeptide-12.

2. The composition of claim 1, wherein the amino acid sequence is GPHGVREA (SEQ ID NO: 1).

3. The composition of claim 1, comprising a high molecular weight (MW) hyaluronic acid (HA) or derivative thereof, wherein the high MW HA has a molecular weight of at least 0.5 MDa.

4. The composition of claim 3, wherein the high MW HA or derivative thereof has a molecular weight of about 1 MDa to 4 MDa.

5. The composition of claim 3, wherein the high MW HA or derivative thereof comprises sodium hyaluronate.

6. The composition of claim 1, wherein the composition further comprises a synthetic tripeptide.

7. The composition of claim 6, wherein the synthetic tripeptide is tetradecyl aminobutyroylvalylaminobutyric urea trifluoroacetate.

8. The composition of claim 1, wherein the composition comprises a hexapeptide-11.

9. The composition of claim 8, wherein the hexapeptide-11 is present at 0.1 ppm to 500 ppm.

10. The composition of claim 1, wherein the composition comprises lactoferrin.

11. The composition of claim 1, wherein the composition comprises phosphatidylserine.

12. The composition of claim 1, further comprising hexapeptide-11, lactoferrin, or phosphatidylserine, wherein the hexapeptide-11, the lactoferrin, or the phosphatidylserine is encapsulated in a liposome.

13. The composition of claim 1, further comprising Tremella fuciformis extract, hydroxymethoxyphenyl decanone, or a combination thereof.

14. The composition of claim 1, wherein:
the tripeptide-1 is present at 0.1 ppm to 200 ppm;
the hexapeptide-12 is present at 0.1 ppm to 200 ppm; and
the polypeptide is present at 0.1 ppm to 200 ppm.

15. The composition of claim 1, wherein the composition is aqueous.

16. A method for reducing inflammation in skin of an individual, promoting skin repair, treating or restoring aging skin, treating a cutaneous wound, or promoting wound healing or wound closure, the method comprising:
administering to the skin a composition comprising:
a synthetic, isolated, or recombinant polypeptide comprising an amino acid sequence GPHGVREA (SEQ ID NO:1), GDGDGASA (SEQ ID NO: 2), GPMGPSGP (SEQ ID NO: 3), GLGPGARA (SEQ ID NO: 4), GPQGFQGP (SEQ ID NO: 5), GPMGPRGP (SEQ ID NO: 6), or GPGKNGDD (SEQ ID NO: 7), wherein the polypeptide comprises no more than 20 amino acids;
a tripeptide-1; and
a hexapeptide-12.

17. The method of claim 16, wherein administering the composition to the skin achieves at least one of the following:
reduces expression of nitric oxide synthase 2 (NOS2), tumor necrosis factor (TNF), interleukin 12 (IL-12b), or cluster of differentiation-80 (CD80), or a combination thereof;
increases CD44 expression;
increases the production of tropoelastin or microfibrillar-associated protein 4 (MFAP4);
upregulates expression of S100A2, LAMA3, MERTK, or ITGB4;
upregulates gene expression in one or more pathways related to extracellular matrix (ECM) regulation;
reduces redness of skin of an individual after applying the composition to the skin;
increases skin hydration in skin of an individual after applying the composition to the skin;
reduces or reverses signs of solar elastosis;
reduces appearance of a bruise, an aging spot, or a wrinkle;
upregulates hyaluronic acid synthase (HAS) expression;
promotes hyaluronic acid synthesis;
increases elastin production in skin; or
downregulates hyaluronidase expression.

18. The method of claim 16, wherein the administering is performed 1, 2, 3, 4, 5, 6, 7, or 8 times a day.

19. The method of claim 16, wherein the composition comprises hexapeptide-11, lactoferrin, phosphatidylserine, Tremella fuciformis extract, hydroxymethoxyphenyl decanone, tetradecyl aminobutyroylvalylaminobutyric urea trifluoroacetate, or any combination thereof.

20. The method of claim 16, wherein the polypeptide comprises the amino acid sequence GPHGVREA (SEQ ID NO:1).


SEQUENCE LISTING (13 sequences)

SEQ ID NO: 1 - protein, 8 aa, synthetic construct
GPHGVREA

SEQ ID NO: 2 - protein, 8 aa, synthetic construct
GDGDGASA

SEQ ID NO: 3 - protein, 8 aa, synthetic construct
GPMGPSGP

SEQ ID NO: 4 - protein, 8 aa, synthetic construct
GLGPGARA

SEQ ID NO: 5 - protein, 8 aa, synthetic construct
GPQGFQGP

SEQ ID NO: 6 - protein, 8 aa, synthetic construct
GPMGPRGP

SEQ ID NO: 7 - protein, 8 aa, synthetic construct
GPGKNGDD

SEQ ID NO: 8 - protein, 6 aa, synthetic construct
IGVAPG

SEQ ID NO: 9 - protein, 6 aa, synthetic construct
VGVAPG

SEQ ID NO: 10 - protein, 8 aa, synthetic construct
  source 1..8: note = N-terminus has Hydrogen, C-terminus has OH group
GPMGPXGP

SEQ ID NO: 11 - protein, 6 aa, synthetic construct
VGVTAG

SEQ ID NO: 12 - protein, 6 aa, synthetic construct
  source 1..6: note = N-terminus has palmitoyl hexapeptide-12
VGVAPG

SEQ ID NO: 13 - protein, 6 aa, synthetic construct
  source 1..6: note = N-terminus has myristoyl hexapeptide-12
VGVAPG